(19) United States
(12) Patent Application Publication
(10) Pub. No.: US 2015/0240247 A1
(43) Pub. Date: Aug. 27, 2015

(54) TROPHIC CONVERSION OF PHOTOAUTOTROPHIC BACTERIA FOR IMPROVED DIURNAL PROPERTIES

(71) Applicant: THE REGENTS OF THE UNIVERSITY OF CALIFORNIA, Oakland, CA (US)

(72) Inventors: Shota Atsumi, Davis, CA (US); Michael R. Connor, Boston, MA (US); Jordan T. McEwen, Davis, CA (US)

(21) Appl. No.: 14/431,751

(22) PCT Filed: Sep. 27, 2013
(86) PCT No.: PCT/US2013/062462

§ 371 Date: Mar. 26, 2015

Related U.S. Application Data
(60) Provisional application No. 61/707,848, filed on Sep. 28, 2012.

(51) Int. Cl. C12N 15/74; C12P 5/00; C12P 7/56; C12P 13/14; C12P 7/48; C12P 7/46; C12P 23/00; C12P 7/42; C12P 13/00; C12P 7/64; C12P 33/00; C12P 19/58; C12P 7/06; C12P 7/28; C12P 7/16; C12P 7/04; C12P 7/24; C12P 5/02; C12P 7/62; C12P 7/18

(52) U.S. Cl. CPC C12N 15/74; C12P 7/18; C12P 5/007; C12P 7/56; C12P 13/14; C12P 7/48; C12P 7/46; C12P 23/00; C12P 7/42; C12P 13/001; C12P 7/6409; C12P 33/00; C12P 19/58; C12P 7/06; C12P 7/28; C12P 7/16; C12P 7/04; C12P 7/24; C12P 5/02; C12P 7/62; C12P 5/026

(57) ABSTRACT

The present disclosure relates generally to the growth of recombinant bacterial cells of photoautotrophic species under diurnal conditions. In particular, the present disclosure relates to isolated bacterial cells of photoautotrophic species having increased growth under diurnal conditions by expression of a sugar transporter protein and methods of use thereof.

### CROSS-REFERENCE TO RELATED APPLICATION(S)

The present application claims priority from U.S. Provisional Application No. 61/707,848, filed Sep. 28, 2012, the content of which is incorporated herein by reference in its entirety for all purposes.

### FIELD

The present disclosure relates generally to the growth of recombinant bacterial cells of photoautotrophic species under diurnal conditions. In particular, the present disclosure relates to isolated bacterial cells of photoautotrophic species having increased growth under diurnal conditions by expression of a sugar transporter protein and methods of use thereof.

### BACKGROUND

According to the US Energy Information Administration (EIA, 2007), world energy-related CO₂ emissions in 2004 were 26,922 million metric tons and increased 26.7% from 1990. As a result, atmospheric levels of CO₂have increased by about 25% over the past 150 years. Thus, it has become increasingly important to develop new technologies to reduce CO₂ emissions.

The world is also facing costly gas and oil and limited reserves of these precious resources. Biofuels have been recognized as an alternative energy source. While efforts have been made to improve various biofuel production methods, further developments are needed.

One solution to the above problems is to utilize plant biomass for the production of biofuels. However, plant productivity has a low yield of conversion of solar energy to biomass and biofuels, due to limitations in CO₂ diffusion and sequestration, growing season, and solar energy collection over the course of the year. A higher energy conversion is achieved by photosynthetic microorganisms such as microalgae and cyanobacteria. It has been previously reported that the cyanobacteria S. elongatus can be engineered to grow on exogenous glucose with light energy by incorporation of a glucose transporter. However, these transformed strains were not stable and required the addition of a chemical inhibitor of photosynthesis, meaning that glucose utilization and photosynthesis were incompatible (Zhang et al., FEMS Microbiology Letters 161 (1998) 285-292). Another problem with S. elongatus cyanobacteria is that growth and biofuel production is completely dependent on light energy, which does not allow it to grow or produce biofuels in the absence of light. Thus, the daily biomass and biofuel production of S. elongatus is limited to sunlight hours, which ranges from 9 to 16 hours a day, thus leading to reduced biofuel productivity and increased production costs.

Accordingly there is a need for bacteria of heretofore photoautotrophic species, such as cyanobacteria, that can utilize sugar substrates to grow under diurnal conditions to continually produce biofuels 24 hours a day.

### BRIEF SUMMARY

In order to meet the above needs, the present disclosure provides recombinant bacterial cells of photoautotrophic species with increased growth on a sugar substrate under diurnal conditions, methods for increasing growth of the recombinant bacterial cells on a sugar substrate under diurnal conditions, and methods for use of the recombinant bacterial cells to produce commodity chemicals. Moreover, the present disclosure is based, at least in part, on the surprising discovery that bacterial cells of a photoautotrophic species engineered to express a recombinant sugar transporter protein (e.g., glucose transporter protein, sucrose transporter protein, or xylose transporter protein) can thrive on an exogenous sugar substrate under diurnal conditions, and especially during the dark or night phase of a day/night diurnal cycle. Advantageously, expression of the recombinant sugar transporter allows the recombinant bacterial cells to utilize exogenous sugar substrate for biomass production under diurnal conditions. Additionally, the recombinant bacterial cells do not require an adjustment period to begin utilizing the sugar substrate. Advantageously, utilization of an exogenous sugar substrate is compatible with and compliments photosynthesis, and thus the recombinant bacterial cells of the present disclosure do not require a chemical inhibitor of photosynthesis. Additionally, the recombinant bacterial cells of the present disclosure do not require a 24 hour light cycle (i.e., continual light input) in order to continually grow, which reduces the production costs of growing the bacterial cells. As such, the recombinant bacterial cells of the present disclosure are able to continually produce commodity chemicals, such as biofuels, 24 hours a day, which increases the productivity of the recombinant bacterial cells and reduces the production costs of commodity chemicals.

Accordingly, one aspect of the present disclosure relates to an isolated bacterial cell of a photoautotrophic species containing a recombinant polynucleotide encoding a galactose transporter protein, where expression of the galactose transporter protein results in transport of glucose into the bacterial cell to increase growth of the bacterial cell on glucose under dark or diurnal conditions as compared to a corresponding photoautotrophic bacterial cell lacking the recombinant polynucleotide. In certain embodiments, the recombinant polynucleotide encodes a galactose transporter protein selected from a bacterial galP transporter protein, a eukaryotic galP transporter protein, a fungal galP transporter protein, a mammalian galP transporter protein, a bacterial Major Facilitator Superfamily (MFS) transporter protein, a eukaryotic MFS transporter protein, a fungal MFS transporter protein, a mammalian MFS transporter protein, a bacterial ATP-Binding Cassette Superfamily (ABC) transporter protein, a eukaryotic ABC transporter protein, a fungal ABC transporter protein, a mammalian ABC transporter protein, a bacterial Phosphotransferase System (PTS) transporter protein, a eukaryotic PTS transporter protein, a fungal PTS transporter protein, a mammalian PTS transporter protein, and a homolog thereof. In certain embodiments, the recombinant polynucleotide encodes an E. coli galP transporter protein.

Another aspect of the present disclosure relates to an isolated bacterial cell of a photoautotrophic species containing a recombinant polynucleotide encoding a disaccharide sugar transporter protein, where expression of the disaccharide sugar transporter protein results in transport of a disaccharide sugar into the bacterial cell to increase growth of the bacterial cell on the disaccharide sugar under dark or diurnal conditions as compared to a corresponding photoautotrophic bacterial cell lacking the recombinant polynucleotide. In certain embodiments, the recombinant polynucleotide encodes a disaccharide sugar transporter protein selected from a sucrose transporter protein, a lactose transporter protein, a lactulose transporter protein, a maltose transporter protein, a trehalose transporter protein, a cellobiose transporter protein, and a homolog thereof. In certain embodiments, the recombinant polynucleotide encodes a sucrose transporter protein. In certain embodiments, the sucrose transporter protein is selected from an E. coli CscB sucrose transporter protein, a B. subtilis SacP transporter protein, a Brassica napus Sut1 transporter protein, a Juglans regia Sut1 transporter protein, an Arabidopsis thaliana Suc6 transporter protein, an Arabidopsis thaliana SUT4 transporter protein, a Drosophila melanogaster Slc45-1 transporter protein, and a Dickeya dadantii ScrA transporter protein. In certain embodiments, the sucrose transporter protein is an E. coli CscB sucrose transporter protein. In certain embodiments that may be combined with any of the preceding embodiments, the bacterial cell further contains at least one additional recombinant polynucleotide encoding a fructokinase protein. In certain embodiments, the fructokinase protein is selected from an E. coli CscK fructokinase protein, a Lycopersicon esculentum Frk1 fructokinase protein, a Lycopersicon esculentum Frk2 fructokinase protein, a H. sapiens KHK fructokinase protein, an A. thaliana FLN-1 fructokinase protein, an A. thaliana and FLN-2 fructokinase protein, a Yersinia pestis biovar Microtus str. 91001 NagC 1 fructokinase protein, a Yersinia pseudotuberculosis YajF fructokinase protein, and a Natronomonas pharaonis Suk fructokinase protein. In certain embodiments, the fructokinase protein is an E. coli CscK fructokinase protein.

Another aspect of the present disclosure relates to an isolated bacterial cell of a photoautotrophic species containing a recombinant polynucleotide encoding a xylose transporter protein, where expression of the xylose transporter protein results in transport of xylose into the bacterial cell to increase growth of the bacterial cell on xylose under dark or diurnal conditions as compared to a corresponding photoautotrophic bacterial cell lacking the recombinant polynucleotide. In certain embodiments, the recombinant polynucleotide encodes a xylose transporter protein selected from an E. coli XylE xylose transporter protein, an E. coli xylF/xylG/xylH ABC xylose transporter protein, a Candida intermedia Gxf1 transporter protein, a Pichia stipitis Sut1 transporter protein, and an A. thaliana At5g59250 transporter protein. In certain embodiments, the recombinant polynucleotide encodes an E. coli XylE xylose transporter protein. In certain embodiments that may be combined with any of the preceding embodiments, the bacterial cell further contains at least one additional recombinant polynucleotide encoding a xylose isomerase. In certain embodiments that may be combined with any of the preceding embodiments, the bacterial cell further contains at least one additional recombinant polynucleotide encoding a xylulokinase. In certain embodiments that may be combined with any of the preceding embodiments, the bacterial cell further contains a second recombinant polynucleotide encoding a xylose isomerase and a third recombinant polynucleotide encoding a xylulokinase. In certain embodiments that may be combined with any of the preceding embodiments, the recombinant polynucleotide further encodes a xylose isomerase and a xylulokinase. In certain embodiments that may be combined with any of the preceding embodiments, the xylose isomerase is selected from an E. coli XylA xylose isomerase, an A. thaliana AT5G57655 xylose isomerase, an Aspergillus niger XyrA xylose isomerase, and a Hypocrea jecorina Xyl1 xylose isomerase. In certain embodiments that may be combined with any of the preceding embodiments, the xylose isomerase is an E. coli XylA xylose isomerase. In certain embodiments that may be combined with any of the preceding embodiments, the xylulokinase is selected from an E. coli XylB xylulokinase, an Arabidopsis thaliana XK-1 xylulokinase, an Arabidopsis thaliana XK-2 xylulokinase, an E. coli LynK xylulokinase, a Streptomyces coelicolor SCO1170 xylulokinase, a Pseudomonas aeruginosa MtlY xylulokinase, a Yersinia pseudotuberculosis SgbK xylulokinase, and an E. coli AtlK xylulokinase. In certain embodiments that may be combined with any of the preceding embodiments, the xylulokinase is an E. coli XylB xylulokinase.

In certain embodiments that may be combined with any of the preceding embodiments, the recombinant polynucleotide and/or at least one additional recombinant polynucleotide is stably integrated into the genome of the bacterial cell. In certain embodiments that may be combined with any of the preceding embodiments, the bacterial cell further contains at least one additional recombinant polynucleotide encoding a sugar transport protein, where expression of the sugar transporter protein results in transport of sugar into the bacterial cell. In certain embodiments that may be combined with any of the preceding embodiments, the sugar is selected from a hexose, galactose, glucose, fructose, mannose, a disaccharide, sucrose, lactose, lactulose, maltose, trehalose, cellobiose, a pentose, xylose, arabinose, ribose, ribulose, and xylulose. In certain embodiments that may be combined with any of the preceding embodiments, the bacterial cell further contains the proteins necessary for the bacterial cell to produce at least one commodity chemical. In certain embodiments, the bacterial cell produces the at least one commodity chemical. In certain embodiments, the bacterial cell continually produces the at least one commodity chemical under diurnal conditions. In certain embodiments that may be combined with any of the preceding embodiments, the commodity chemical is selected from a polymer, 2,3-butanediol, 1,3-propandiol, 1,4-butanediol, polyhydroxyalkanoate, polyhydroxybutyrate, isoprene, lactate, succinate, glutamate, citrate, malate, 3-hydroxypropionate, ascorbate, sorbitol, an amino acid, hydroxybutyrate, a carotenoid, lycopene, β-carotene, a pharmaceutical intermediate, a polyketide, a statin, an omega-3 fatty acid, an isoprenoid, a steroid, an antibiotic, erythromycin, a soprenoid, a steroid, erythromycin, and combinations thereof. In certain embodiments that may be combined with any of the preceding embodiments, the commodity chemical is a biofuel selected from an alcohol, ethanol, propanol, isopropanol, acetone, butanol, isobutanol, 2-methyl-1-butanol, 3-methyl-1-butanol, phenylethanol, a fatty alcohol, isopentenol, an aldehyde, acetylaldehyde, propionaldehyde, butryaldehyde, isobutyraldehyde, 2-methyl-1-butanal, 3-methyl-1-butanal, phenylacetaldehyde, a fatty aldehyde, a hydrocarbon, an alkane, an alkene, an isoprenoids, a fatty acid, a wax ester, an ethyl ester, hydrogen, and combinations thereof. In certain embodiments that may be combined with any of the preceding embodiments, the bacterial cell is selected from cyanobacteria, Acaryochloris, Anabaena, Arthrospira, Cyanothece, Gleobacter, Microcystis, Nostoc, Prochlorococcus, Synechococcus, Synechococcus elongatus, S. elongatus PCC7942, Synechocystis, Thermosynechococcus, Trichodesmium, purple sulfur bacteria, Chromatiaceae, Ectothiorhodospiraceae, purple non-sulfur bacteria, Acetobacteraceae, Bradyrhizobiaceae, Comamonadaceae, Hyphomicrobiaceae, Rhodobacteraceae, Rhodobiaceae, Rhodocyclaceae, Rhodospirillaceae, green non-sulfur bacteria, and Chloroflexaceae.

Another aspect of the present disclosure includes a method of increasing bacterial growth, by providing a bacterial cell of a photoautotrophic species containing a recombinant polynucleotide encoding a sugar transporter protein; and culturing the bacterial cell with a sugar substrate under conditions whereby the recombinant polynucleotide is expressed, where expression of the recombinant polynucleotide results in transport of the sugar substrate into the bacterial cell to increase cell growth on sugar under dark or diurnal conditions as compared to cell growth of a corresponding photoautotrophic bacterial cell lacking the recombinant polynucleotide.

Another aspect of the present disclosure includes a method of increasing bacterial cell density under dark or diurnal conditions, by providing a bacterial cell of a photoautotrophic species containing a recombinant polynucleotide encoding a sugar transporter protein; and culturing the bacterial cell with a sugar substrate under conditions whereby the recombinant polynucleotide is expressed, where expression of the recombinant polynucleotide results in transport of the sugar substrate into the bacterial cell to increase cell density under dark or diurnal conditions as compared to a corresponding photoautotrophic bacterial cell lacking the recombinant polynucleotide.

Another aspect of the present disclosure includes a method of increasing bacterial biomass production under dark or diurnal conditions, by providing a bacterial cell of a photoautotrophic species containing a recombinant polynucleotide encoding a sugar transporter protein; and culturing the bacterial cell with a sugar substrate under conditions whereby the recombinant polynucleotide is expressed, where expression of the recombinant polynucleotide results in transport of the sugar substrate into the bacterial cell to increase biomass production under dark or diurnal conditions as compared to a corresponding photoautotrophic bacterial cell lacking the recombinant polynucleotide.

Another aspect of the present disclosure includes a method of producing at least one commodity chemical, by providing a bacterial cell of a photoautotrophic species containing a recombinant polynucleotide encoding a sugar transporter protein; culturing the bacterial cell with a sugar substrate under conditions whereby the recombinant polynucleotide is expressed and at least one commodity chemical is produced; and collecting the at least one commodity chemical, where expression of the recombinant polynucleotide results in transport of the sugar substrate into the bacterial cell. In certain embodiments, the bacterial cell contains the proteins necessary for the bacterial cell to produce the at least one commodity chemical.

In certain embodiments that may be combined with any of the preceding embodiments, the recombinant polynucleotide encodes a sugar transporter protein selected from a hexose sugar transporter protein, a galactose transporter protein, a glucose transporter protein, a fructose transporter protein, a mannose transporter protein, a Major Facilitator Superfamily (MFS) transporter protein, an ATP-Binding Cassette Superfamily (ABC) transporter protein, a Phosphotransferase System (PTS) transporter protein, a disaccharide sugar transporter protein, a sucrose transporter protein, a lactose transporter protein, a lactulose transporter protein, a maltose transporter protein, a trehalose transporter protein, a cellobiose transporter protein, a pentose transporter protein, a xylose transporter protein, an arabinose transporter protein, a ribose transporter protein, a ribulose transporter protein, and a xylulose transporter protein. In certain embodiments, the bacterial cell is cultured with a sugar selected from a hexose, galactose, glucose, fructose, mannose, a disaccharide, sucrose, lactose, lactulose, maltose, trehalose, cellobiose, a pentose, xylose, arabinose, ribose, ribulose, and xylulose. In certain embodiments that may be combined with any of the preceding embodiments, the recombinant polynucleotide encodes a galactose transporter protein. In certain embodiments, the galactose transporter protein is selected from a bacterial galP transporter protein, a eukaryotic galP transporter protein, a fungal galP transporter protein, a mammalian galP transporter protein, a bacterial MFS transporter protein, a eukaryotic MFS transporter protein, a fungal MFS transporter protein, a mammalian MFS transporter protein, a bacterial PTS transporter protein, a eukaryotic PTS transporter protein, a fungal PTS transporter protein, and a mammalian PTS transporter protein. In certain embodiments, the galactose transporter protein is an E. coli galP transporter protein. In certain embodiments that may be combined with any of the preceding embodiments, the galactose transporter protein transports glucose into the bacterial cell. In certain embodiments that may be combined with any of the preceding embodiments, the bacterial cell is cultured with glucose. In certain embodiments that may be combined with any of the preceding embodiments, the recombinant polynucleotide encodes a disaccharide sugar transporter protein. In certain embodiments, the disaccharide sugar transporter protein is selected from a sucrose transporter protein, a lactose transporter protein, a lactulose transporter protein, a maltose transporter protein, a trehalose transporter protein, a cellobiose transporter protein, and a homolog thereof. In certain embodiments, the disaccharide sugar transporter protein is a sucrose transporter protein. In certain embodiments, the sucrose transporter protein is selected from an E. coli CscB sucrose transporter protein, a B. subtilis SacP transporter protein, a Brassica napus Sut1 transporter protein, a Juglans regia Sut1 transporter protein, an Arabidopsis thaliana Suc6 transporter protein, an Arabidopsis thaliana SUT4 transporter protein, a Drosophila melanogaster Slc45-1 transporter protein, and a Dickeya dadantii ScrA transporter protein. In certain embodiments, the sucrose transporter protein is an E. coli CscB sucrose transporter protein. In certain embodiments that may be combined with any of the preceding embodiments, the bacterial cell further contains at least one additional recombinant polynucleotide encoding a fructokinase protein. In certain embodiments, the fructokinase is selected from an E. coli CscK fructokinase protein, a Lycopersicon esculentum Frk1 fructokinase protein, a Lycopersicon esculentum Frk2 fructokinase protein, a H. sapiens KHK fructokinase protein, an A. thaliana FLN-1 fructokinase protein, an A. thaliana and FLN-2 fructokinase protein, a Yersinia pestis biovar Microtus str. 91001 NagC1 fructokinase protein, a Yersinia pseudotuberculosis YajF fructokinase protein, and a Natronomonas pharaonis Suk fructokinase protein. In certain embodiments, the fructokinase protein is an E. coli CscK fructokinase protein. In certain embodiments that may be combined with any of the preceding embodiments, the bacterial cell further contains the proteins necessary to convert the disaccharide sugar into its corresponding monosaccharides. In certain embodiments that may be combined with any of the preceding embodiments, the bacterial cell is cultured with a sugar selected from a disaccharide sugar, sucrose, lactose, lactulose, maltose, trehalose, and cellobiose. In certain embodiments that may be combined with any of the preceding embodiments, the recombinant polynucleotide encodes a xylose transporter protein. In certain embodiments, the xylose transporter protein selected from an E. coli XylE xylose transporter protein, an E. coli xylF/xylG/xylH ABC xylose transporter protein, a Candida intermedia Gxf1 transporter protein, a Pichia stipitis Sut1 transporter protein, and an A. thaliana At5g59250transporter protein. In certain embodiments, the xylose transporter protein is an E. coli XylE xylose transporter protein. In certain embodiments that may be combined with any of the preceding embodiments, the bacterial cell further contains at least one additional recombinant polynucleotide encoding a xylose isomerase. In certain embodiments that may be combined with any of the preceding embodiments, the bacterial cell further contains at least one additional recombinant polynucleotide encoding a xylulokinase. In certain embodiments that may be combined with any of the preceding embodiments, the bacterial cell further contains a second recombinant polynucleotide encoding a xylose isomerase and a third recombinant polynucleotide encoding a xylulokinase. In certain embodiments that may be combined with any of the preceding embodiments, the recombinant polynucleotide further encodes a xylose isomerase and a xylulokinase. In certain embodiments that may be combined with any of the preceding embodiments, the xylose isomerase is selected from an E. coli XylA xylose isomerase, an A. thaliana AT5G57655 xylose isomerase, an Aspergillus niger XyrA xylose isomerase, and a Hypocrea jecorina Xyl1 xylose isomerase. In certain embodiments that may be combined with any of the preceding embodiments, the xylose isomerase is an E. coli XylA xylose isomerase. In certain embodiments that may be combined with any of the preceding embodiments, the xylulokinase is selected from an E. coli XylB xylulokinase, an Arabidopsis thaliana XK-1 xylulokinase, an Arabidopsis thaliana XK-2 xylulokinase, an E. coli LynK xylulokinase, a Streptomyces coelicolor SCO1170 xylulokinase, a Pseudomonas aeruginosa MtlY xylulokinase, a Yersinia pseudotuberculosis SgbK xylulokinase, and an E. coli AtlK xylulokinase. In certain embodiments that may be combined with any of the preceding embodiments, the xylulokinase is an E. coli XylB xylulokinase. In certain embodiments that may be combined with any of the preceding embodiments, the recombinant polynucleotide and/or at least one additional recombinant polynucleotide is stably integrated into the genome of the bacterial cell. In certain embodiments that may be combined with any of the preceding embodiments, the bacterial cell further contains at least one additional recombinant polynucleotide encoding a second sugar transport protein, where expression of the second sugar transporter protein results in transport of a second sugar substrate into the bacterial cell. In certain embodiments that may be combined with any of the preceding embodiments, the bacterial cell continually produces the at least one commodity chemical. In certain embodiments, the at least one commodity chemical is continually produced 24 hours a day. In certain embodiments that may be combined with any of the preceding embodiments, he commodity chemical is selected from a polymer, 2,3-butanediol, 1,3-propandiol, 1,4-butanediol, polyhydroxyalkanoate, polyhydroxybutyrate, isoprene, lactate, succinate, glutamate, citrate, malate, 3-hydroxypropionate, ascorbate, sorbitol, an amino acid, hydroxybutyrate, a carotenoid, lycopene, β-carotene, a pharmaceutical intermediate, a polyketide, a statin, an omega-3 fatty acid, an isoprenoid, a steroid, an antibiotic, erythromycin, a soprenoid, a steroid, erythromycin, a biofuel, and combinations thereof. In certain embodiments that may be combined with any of the preceding embodiments, the commodity chemical is a biofuel selected from an alcohol, ethanol, propanol, isopropanol, acetone, butanol, isobutanol, 2-methyl-1-butanol, 3-methyl-1-butanol, phenylethanol, a fatty alcohol, isopentenol, an aldehyde, acetylaldehyde, propionaldehyde, butryaldehyde, isobutyraldehyde, 2-methyl-1-butanal, 3-methyl-1-butanal, phenylacetaldehyde, a fatty aldehyde, a hydrocarbon, an alkane, an alkene, an isoprenoids, a fatty acid, a wax ester, an ethyl ester, hydrogen, and combinations thereof. In certain embodiments that may be combined with any of the preceding embodiments, the bacterial cell is selected from cyanobacteria, Acaryochloris, Anabaena, Arthrospira, Cyanothece, Gleobacter, Microcystis, Nostoc, Prochlorococcus, Synechococcus, Synechococcus elongatus, S. elongatus PCC7942, Synechocystis, Thermosynechococcus, Trichodesmium, purple sulfur bacteria, Chromatiaceae, Ectothiorhodospiraceae, purple non-sulfur bacteria, Acetobacteraceae, Bradyrhizobiaceae, Comamonadaceae, Hyphomicrobiaceae, Rhodobacteraceae, Rhodobiaceae, Rhodocyclaceae, Rhodospirillaceae, green non-sulfur bacteria, and Chloroflexaceae.

### BRIEF DESCRIPTION OF THE DRAWINGS

FIG. 1A depicts a schematic representation of the integration of a glucose transporter gene into the genome of S. elongatus. FIG. 1B depicts a growth curve of the S. elongates galP strain (circles) and the wild-type strain (squares) cultured with and without 5 g/L glucose. FIG. 1C depicts a growth curve of the S. elongates Glut 1 strain (triangles), glcP strain (diamonds), and the wild-type strain (squares) cultured with and without glucose. For FIGS. 1B and 1C, open shapes depict growth without 5 g/L glucose, and solid shapes depict growth with 5 g/L glucose. FIG. 1D depicts a growth curve of the S. elongates galP strain (circles) and the wild-type strain (square) with and without the glgC deletion. For FIG. 1D, samples were cultured with 5 g/L glucose. Solid shapes depict strain containing the glgC gene, and “x” shapes depict strains having the glgC deletion. For FIGS. 1B-1D, white areas indicate light cycles and shaded areas indicate dark cycles. Error bars represent standard deviation in triplicate.

FIG. 2A depicts a schematic representation of a recombination event to delete the glgC gene with the pAL82 plasmid to create the S. elongatus glgC deletion strain. Bars indicate homologous regions for recombination. Arrowheads indicate primers used for the verification of the recombination. FIG. 2B depicts PCR confirmation of correct recombinants. PCR was performed with primers GR050 and IM581 (Lane 1-3, product size: 1.7 kb); and IM573 and GR015 (Lane 4-6, product size: 2.2 kb), using genomic DNA of the S. elongatus wild type strain (lanes 1 and 4), AL535 strain (lanes 2 and 5), and AL536 strain (lanes 3 and 6) as templates.

FIG. 3A-E depicts the characterization of the S. elongates galP strain. All samples were grown in BG-11 media with 5 g/L glucose under continuous light. FIG. 3A depicts a confocal microscope image (left) and a transmitted light microscope image (right) of the S. elongates wild-type strain. FIG. 3B depicts a confocal microscope image of the S. elongates gfp strain (left), and the S. elongates galP-gfp strain (right). FIG. 3C depicts a growth curve of the S. elongates galP-gfp strain cultured in the presence of varying amounts of IPTG. Circles represent no IPTG, squares represent 0.01 mM IPTG; triangles represent 0.1 mM IPTG; and diamonds represent 1.0 mM IPTG. FIG. 3D depicts fluorescence analysis of the S. elongates galP-gfp strain during the course of the growth curve analysis depicted in FIG. 3C. The Y-axis indicates GFP fluorescence intensity divided with OD₇₃₀. FIG. 3E depicts a growth curve of the S. elongates galP strain (without gfp; closed shape) and the wild-type strain (open shape) cultured in the presence of various concentrations of sodium bicarbonate (NaHCO₃). Squares represent no bicarbonate; circles represent 5 mM bicarbonate; triangles represent 10 mM bicarbonate; and inverted triangles represent 20 mM bicarbonate. Error bars represent standard deviation in triplicate.

FIG. 4A depicts a schematic representation of the integration of the sucrose degradation pathway into the genome of S. elongatus. FIG. 4B depicts a synthetic sucrose degradation pathway in S. elongatus. Grey arrows indicate steps catalyzed by heterologous enzymes. “PPP” corresponds to the pentose phosphate pathway. FIG. 4C depicts a growth curve of the S. elongatus cscB-cscK strain (circles) and the wild-type strain (squares). Empty shapes represent cells cultured without 5 g/L sucrose, and solid shapes represent cells cultured with 5 g/L sucrose. White areas indicate light cycles and shaded areas indicate dark cycles. Error bars represent standard deviation in triplicate.

FIG. 5A depicts a schematic representation of the integration of the xylose degradation pathway into the genome of S. elongatus. FIG. 5B depicts a synthetic xylose degradation pathway in S. elongatus. Grey arrows indicate steps catalyzed by heterologous enzymes. FIG. 5C depicts a growth curve of the S. elongatus xylEAB strain (circles), xylE strain (squares), and wild-type strain (triangles). Empty shapes represent cells cultured without 5g/L xylose and solid shapes represent cells cultured with 5 g/L xylose. White areas indicate light cycles and shaded areas indicate dark cycles. Error bars represent standard deviation in triplicate.

### DETAILED DESCRIPTION

The present disclosure is based, at least in part, on the surprising discovery that recombinant expression of a sugar transporter protein, such as a glucose transporter protein, a sucrose transporter protein, or a xylose transporter protein, allows bacterial cells of a photoautotrophic species to grow in the absence of light and thus to continually produce commodity chemicals, such as biofuels, 24 hours a day. Advantageously, the recombinant bacterial cells of the present disclosure do not require a chemical inhibitor of photosynthesis nor do they require continual light input to grow, thus reducing production costs.

Certain aspects of the present disclosure provide isolated bacterial cells of a photoautotrophic species containing a recombinant polynucleotide encoding a galactose transporter protein, where expression of the galactose transporter protein results in transport of glucose into the bacterial cell to increase growth of the bacterial cell on glucose under dark or diurnal conditions as compared to a corresponding photoautotrophic bacterial cell of the same species lacking the recombinant polynucleotide. Other aspects of the present disclosure provide isolated bacterial cells of a photoautotrophic species containing a recombinant polynucleotide encoding a disaccharide sugar transporter protein, where expression of the disaccharide sugar transporter protein results in transport of a disaccharide sugar into the bacterial cell to increase growth of the bacterial cell on the disaccharide sugar under dark or diurnal conditions as compared to a corresponding photoautotrophic bacterial cell of the same species lacking the recombinant polynucleotide. Other aspects of the present disclosure provide isolated bacterial cells of a photoautotrophic species containing a recombinant polynucleotide encoding a xylose transporter protein, where expression of the xylose transporter protein results in transport of xylose into the bacterial cell to increase growth of the bacterial cell on xylose under dark or diurnal conditions as compared to a corresponding photoautotrophic bacterial cell of the same species lacking the recombinant polynucleotide. In certain embodiments, the isolated bacterial cells produce a commodity chemical.

Other aspects of the present disclosure provide methods of increasing bacterial growth by providing a bacterial cell of a photoautotrophic species containing a recombinant polynucleotide encoding a sugar transporter protein; and culturing the bacterial cell with a sugar substrate under conditions whereby the recombinant polynucleotide is expressed, where expression of the recombinant polynucleotide results in transport of the sugar substrate into the bacterial cell to increase cell growth on sugar under dark or diurnal conditions as compared to cell growth of a corresponding photoautotrophic bacterial cell of the same species lacking the recombinant polynucleotide. Other aspects of the present disclosure provide methods of increasing bacterial cell density or biomass production under dark or diurnal conditions by providing a bacterial cell of a photoautotrophic species containing a recombinant polynucleotide encoding a sugar transporter protein; and culturing the bacterial cell with a sugar substrate under conditions whereby the recombinant polynucleotide is expressed, where expression of the recombinant polynucleotide results in transport of the sugar substrate into the bacterial cell to increase cell density or biomass production under dark or diurnal conditions as compared to a corresponding photoautotrophic bacterial cell of the same species lacking the recombinant polynucleotide.

Further aspects of the present disclosure provide methods of producing at least one commodity chemical by providing a bacterial cell of a photoautotrophic species containing a recombinant polynucleotide encoding a sugar transporter protein; culturing the bacterial cell with a sugar substrate under conditions whereby the recombinant polynucleotide is expressed and at least one commodity chemical is produced; and collecting the at least one commodity chemical, where expression of the recombinant polynucleotide results in transport of the sugar substrate into the bacterial cell. In certain embodiments, the bacterial cell contains the proteins necessary for the bacterial cell to produce the at least one commodity chemical.

### Isolated Bacterial Cells

Certain aspects of the present disclosure relate to recombinant bacterial cells of a photoautotrophic species that are able to grow on sugars, such as galactose, sucrose, or xylose.

As used herein, the term “photoautotrophic bacteria,” “photoautotrophic bacterial cell(s),” “photoautotroph(s),” or “cell(s) of a photoautotrophic species” refers to bacterial cells that carry out photosynthesis to acquire energy and can utilize carbon dioxide as a sole carbon source. Photoautotrophic bacteria use the energy from sunlight to convert carbon dioxide and water into organic materials to be utilized in cellular functions, such as biosynthesis and respiration. Photoautotrophic bacteria are typically Gram-negative rods which obtain their energy from sunlight through the process of photosynthesis. In this process, sunlight energy is used in the synthesis of carbohydrates, which in recombinant photoautotrophs can be further used as intermediates in the synthesis of biofuels. Certain photoautotrophs called anoxygenic photoautotrophs grow only under anaerobic conditions and neither use water as a source of hydrogen nor produce oxygen from photosynthesis. Other photoautotrophic bacteria include oxygenic photoautotrophs. These bacteria are typically cyanobacteria. Oxygenic photoautotrophs use chlorophyll pigments and photosynthesis in photosynthetic processes resembling those in algae and complex plants. During the process, they use water as a source of hydrogen and produce oxygen as a product of photosynthesis.

Suitable isolated bacterial cells of the present disclosure include, without limitation, halobacteria, heliobacteria, purple sulfur bacteria, purple non-sulfur bacteria, green sulfur bacteria, green non-sulfur bacteria, and cyanobacteria. Cyanobacteria typically include types of bacterial rods and cocci, as well as certain filamentous forms. Examples of suitable cyanobacteria include, without limitation, Prochlorococcus, Synechococcus, and Nostocvarious. The cells may contain thylakoids, which are cytoplasmic, platelike membranes containing chlorophyll. The organisms produce heterocysts, which are specialized cells believed to function in the fixation of nitrogen compounds.

Accordingly, in certain embodiments, an isolated cell of the present disclosure is selected from cyanobacteria, Acaryochloris, Anabaena, Arthrospira, Cyanothece, Gleobacter, Microcystis, Nostoc, Prochlorococcus, Synechococcus, Synechococcus elongatus, S. elongatus PCC7942, Synechocystis, Thermosynechococcus, Trichodesmium, purple sulfur bacteria, Chromatiaceae, Ectothiorhodospiraceae, purple non-sulfur bacteria, Acetobacteraceae, Bradyrhizobiaceae, Comamonadaceae, Hyphomicrobiaceae, Rhodobacteraceae, Rhodobiaceae, Rhodocyclaceae, Rhodospirillaceae, green non-sulfur bacteria, and Chloroflexaceae. In still other embodiments, the photoautotrophic bacterial cell is a cyanobacterium. In yet other embodiments, the isolated bacterial cell is Synechococcus. Preferably, the isolated bacterial cell is Synechococcus elongatus. More preferably, the isolated bacterial cell is S. elongatus PCC7942.

Isolated bacterial cells of a photoautotrophic species of the present disclosure are genetically modified in that recombinant polynucleotides have been introduced into the bacterial cells, and as such the genetically modified bacterial cells do not occur in nature. The suitable isolated bacterial cell is one capable of expressing one or more polynucleotide constructs encoding one or more proteins capable of transporting a desired sugar. In preferred embodiments, the one or more proteins include, but are not limited to a Major Facilitator Superfamily (MFS) transporter, a Phosphotransferase System (PTS) transporter, an ATP-Binding Cassette Superfamily (ABC) transporter, a hexose sugar transporter, a galactose transporter, a glucose transporter, a fructose transporter, a mannose transporter, a pentose transporter, a xylose transporter, an arabinose transporter, a ribose transporter, a ribulose transporter, a xylulose transporter, a sucrose transporter, a lactose transporter, a lactulose transporter, a maltose transporter, a trehalose transporter, and a cellobiose transporter. In preferred embodiments, the one or more proteins are capable of transporting sugars, which lead to increased growth, biomass production, and commodity chemical production under diurnal conditions.

“Diurnal condition(s)” or “diurnal cycle(s)” as used herein refers to growth conditions that occur over a daily 24 hour cycle and that include light or daylight phases and dark or night phases.

“Recombinant polynucleotide” or “recombinant nucleic acid” as used herein refers to a polymer of nucleic acids wherein at least one of the following is true: (a) the sequence of nucleic acids is foreign to (i.e., not naturally found in) a given host microorganism; (b) the sequence may be naturally found in a given host microorganism, but is present in an unnatural (e.g., greater than expected) amount; or (c) the sequence of nucleic acids contains two or more subsequences that are not found in the same relationship to each other in nature. For example, regarding instance (c), a recombinant nucleic acid sequence will have two or more sequences from unrelated genes arranged to make a new functional nucleic acid. Specifically, the present disclosure describes the introduction of an expression vector into an isolated bacterial cell, where the expression vector contains a nucleic acid sequence coding for an enzyme that is not normally found in the cell or contains a nucleic acid coding for an enzyme that is normally found in the cell but is under the control of different regulatory sequences. With reference to the isolated bacterial cell's genome, then, the nucleic acid sequence that codes for the protein is recombinant.

“Genetically engineered” or “genetically modified” refers to any isolated bacterial cell of a photoautotrophic species modified by any recombinant DNA or RNA technology. In other words, the isolated bacterial cell has been transfected, transformed, or transduced with a recombinant polynucleotide molecule, and thereby been altered so as to cause the cell to alter expression of a desired protein. Methods and vectors for genetically engineering isolated bacterial cells are well known in the art; for example, various techniques are illustrated in Current Protocols in Molecular Biology, Ausubel et al., eds. (Wiley & Sons, New York, 1988, and quarterly updates). Genetic engineering techniques include but are not limited to expression vectors, targeted homologous recombination, and gene activation (see, for example, U.S. Pat. No. 5,272,071), and trans-activation by engineered transcription factors (see, for example, Segal et al., 1999, Proc Natl Acad Sci USA 96(6):2758-63).

Genetic modifications that result in an increase in gene expression or function can be referred to as amplification, overproduction, overexpression, activation, enhancement, addition, or up-regulation of a gene. More specifically, reference to increasing the action (or activity) of proteins or enzymes discussed herein generally refers to any genetic modification in the photoautotrophic bacterial cells in question that results in increased expression and/or functionality (biological activity) of the proteins or enzymes and includes higher activity of the enzymes (e.g., specific activity or in vivo enzymatic activity), reduced inhibition or degradation of the enzymes, and overexpression of the enzymes. For example, gene copy number can be increased, expression levels can be increased by use of a promoter that gives higher levels of expression than that of the native promoter, or a gene can be altered by genetic engineering or classical mutagenesis to increase the biological activity of an enzyme. Combinations of some of these modifications are also possible.

In general, according to the present disclosure, an increase or a decrease in a given characteristic of a mutant or modified protein (e.g., protein function) is made with reference to the same characteristic of a wild-type (i.e., normal, not modified) protein that is derived from the same organism (i.e., from the same source or parent sequence), and is measured or established under the same or equivalent conditions. Similarly, an increase or decrease in a characteristic of a genetically modified isolated bacterial cell (e.g., expression and/or biological activity of a protein, or production of a product) is made with reference to the same characteristic of a wild-type bacterial cell of the same species, and preferably the same strain, under the same or equivalent conditions. Such conditions include the assay or culture conditions (e.g., medium components, temperature, pH, etc.) under which the activity of the protein (e.g., expression or biological activity) or other characteristic of the isolated bacterial cell is measured, as well as the type of assay used, the host bacterial cell that is evaluated, etc. As discussed above, equivalent conditions are conditions (e.g., culture conditions) which are similar, but not necessarily identical (e.g., some conservative changes in conditions can be tolerated), and which do not substantially change the effect on microbe growth or enzyme expression or biological activity as compared to a comparison made under the same conditions.

Preferably, a genetically modified isolated bacterial cell of a photoautotrophic species that has a genetic modification that increases or decreases the function of a given protein has an increase or decrease, respectively, in the activity (e.g., expression, production and/or biological activity) of the protein, as compared to the activity of the wild-type protein in a corresponding wild-type bacterial cell of the same species lacking the protein, of at least about 2-fold, and more preferably at least about 5-fold, and more preferably at least about 10-fold, and more preferably about 20-fold, and more preferably at least about 30-fold, and more preferably at least about 40-fold, and more preferably at least about 50-fold, and more preferably at least about 75-fold, and more preferably at least about 100-fold, and more preferably at least about 125-fold, and more preferably at least about 150-fold, or any whole integer increment starting from at least about 2-fold (e.g., 3-fold, 4-fold, 5-fold, 6-fold, etc.).

### Sugar Transporter Proteins

Other aspects of the present disclosure relate to isolated bacterial cells of a photoautotrophic species containing a recombinant polynucleotide encoding a sugar transporter protein, such as a galactose transporter protein, a sucrose transporter protein, or a xylose transporter protein.

Sugar transporter proteins of the present disclosure include, without limitation, any transporter protein that is capable of transporting an amount of sugar substrate sufficient to be utilized by a photoautotrophic bacterial cell of the present disclosure without being toxic. Moreover, utilization of the sugar substrate increases the growth, cell density, and/or biomass production of the photoautotrophic bacterial cell under dark and/or diurnal conditions.

Suitable sugar transporter proteins include, without limitation, Major Facilitator Superfamily (MFS) transporter proteins, Phosphotransferase System (PTS) transporter proteins, ATP-Binding Cassette (ABC) Superfamily transporter proteins, hexose sugar transporter proteins, disaccharide sugar transporter proteins, and pentose transporter proteins.

Examples of suitable hexose sugar transporter proteins include without limitation, galactose transporter proteins, such a galP transporter protein, an MFS transporter protein, a PTS transporter protein, and the SyjfF/ytfR/ytfT/ytfQ ABC system of Escherichia coli; glucose transporter proteins, such as the ptsI/ptsH/ptsG/crr PTS system of Escherichia coli, the GLUT1 and GLUT3 MFS transporters of Homo sapiens, and the glcP MFS transporter of Synechocystis sp PCC6803; fructose transporter proteins, such as the GLUT5 MFS transporter of Homo sapiens; mannose transporter proteins, such as the manX/manY/manZ PTS system of Escherichia coli, and glucose-specific transporters that transport mannose.

Examples of suitable disaccharide sugar transporter proteins include, without limitation, sucrose transporter proteins, such as the sacP PTS system of Bacillus subtilis, and the cscB MFS transporter of Escherichia coli EC3132; lactose transporter proteins, such as the lacY MFS transporter from Escherichia coli; lactulose transporter proteins, such as the LacE2 MFS transporter from Streptococcus pneumoniae; maltose transporter proteins, such as the malE/malF/malG/malK ABC system of Escherichia coli; trehalose transporter proteins, such as the TreB PTS proteins from Escherichia coli; and cellobiose transporter proteins, such as the CelD PTS system in Strptococcus pneumonia.

Examples of suitable pentose transporter proteins include, without limitation, xylose transporter proteins, such as the E. coli xylE MFS transporter protein and the xylF/xylG/xylH ABC system of Escherichia coli; arabinose transporter proteins, such as the araJ MFS transporter of Escherichia coli; ribose transporter proteins, such as the Rbs ABC transporter system of Escherichia coli; ribulose transporter proteins; and xylulose transporter proteins, such as the MFS transporter (LKI_{—}09995) of Leuconostoc kimchii.

Suitable sugar transporters proteins also include, without limitation, transporter proteins that have been mutated or altered from their endogenous form so as to enable the transport of an amount of sugar substrate sufficient to be utilized by a photoautotrophic bacterial cell of the present disclosure without being toxic.

Additionally, the sugar transporter proteins described herein can be readily replaced using a homologous protein thereof. “Homologous protein” as used herein refers to a protein that has a polypeptide sequence that is at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to any of the proteins described in this specification or in a cited reference. Homologous proteins retain amino acid residues that are recognized as conserved for the protein. Homologous proteins may have non-conserved amino acid residues replaced or found to be of a different amino acid, or amino acid(s) inserted or deleted, as long as they do not affect or have insignificant effect on the function or activity of the homologous protein. A homologous protein has a function or activity that is essentially the same as the function or activity of any one of the proteins described in this specification or in a cited reference. Homologous proteins may be found in nature or be an engineered mutant thereof.

Galactose Transporter Proteins

In certain embodiments, isolated bacterial cells of the present disclosure contain a recombinant polynucleotide encoding a galactose transporter protein that is capable of transporting glucose into the bacterial cell. Galactose transporter proteins are integral membrane proteins that generally transport galactose into a cell. However, certain galactose transporters, such as the E. coli galactose transporter galP and other members of the Major Facilitator Superfamily (MFS) transporters, have been shown to be capable of also transporting glucose into a cell (Flores N et al Nat Biotech 14(5): 620-623. 1996). Such galactose transporters have been shown to transport low levels of glucose into photoautotrophic bacterial cells, such as Synechococcus elongatus, as compared to levels of glucose that is transported into the cell by a glucose transporter. Without wishing to be bound by theory, it is believed that most chemoheterotrophic microorganisms, such as E. coli, use a large percentage of their glucose uptake for energy consumption (e.g., burning to CO₂—measured by biomass produced vs. sugar consumed) while photoautotrophic bacterial cells, such S. elongatus, generally generate the majority of their energy from photosynthesis, and therefore only use glucose uptake as a source of carbon backbone for biosynthesis. Without wishing to be bound by theory, it is also believed that the transport of high levels of glucose into the cell can be toxic to certain photoautotrophic bacterial cells, such as the cyanobacteria S. elongatus. However, without wishing to be bound by theory, it is further believed that transport of low levels of glucose is not toxic and can be utilized by photoautotrophic bacterial cells, such as the cyanobacteria S. elongatus, to grow during the dark phases of a diurnal cycle.

Galactose transporters may be active transporters that require energy to transport the sugar into the cell. The energy may be supplied by ATP or by co-transporting an ion down its electrochemical gradient. For example, ATP-Binding Cassette (ABC) transporters utilize ATP to transport sugars into a photoautotrophic bacterial cell. Alternatively, a galactose transporter may be a passive transporter that utilizes facilitated diffusion to transport sugar into the cell. In certain embodiments, the galactose transporter is a galP transporter. The galP transporter is a member of the Major Facilitator Superfamily family of transporters. The MTS transporters are sugar-proton (H⁺) symporters that pump the sugar into the bacterial cell cytosol against a concentration gradient by secondary active transport into the cell, using the electrochemical H⁺ gradient.

Another example of a suitable galactose transport system is a phosphotransferase system. Phosphotransferase system (PTS), also known as PEP group translocation, is a distinct method of active transport used by bacterial cells for sugar uptake where the source of energy is from phosphoenolpyruvate (PEP). The PTS system is known as multicomponent system that involves enzymes of the plasma membrane and those in the cytoplasm. An example of this transport is found in E. coli. The PTS system is involved in transporting many sugars into bacterial cells, including galactose, glucose, mannose, fructose, and cellobiose.

Accordingly, in certain embodiments, the galactose transporter protein is selected from a bacterial galP transporter protein, a eukaryotic galP transporter protein, a fungal galP transporter protein, a mammalian galP transporter protein, a bacterial Major Facilitator Superfamily (MFS) transporter protein, a eukaryotic MFS transporter protein, a fungal MFS transporter protein, a mammalian MFS transporter protein, a bacterial ATP-Binding Cassette Superfamily (ABC) transporter protein, a eukaryotic ABC transporter protein, a fungal ABC transporter protein, a mammalian ABC transporter protein, a bacterial Phosphotransferase System (PTS) transporter protein, a eukaryotic PTS transporter protein, a fungal PTS transporter protein, a mammalian PTS transporter protein, and a homolog thereof. In certain preferred embodiments, the galactose transporter protein is an E. coli galP transporter protein, or homologs thereof. In other preferred embodiments, the galactose transporter protein is an E. coli galP transporter protein having an amino acid sequence that is at least at least 70%, at least 75%, at least 80%, at least 85%, at least 90%, at least 91%, at least 92%, at least 93%, at least 94%, at least 95%, at least 96%, at least 97%, at least 98%, or at least 99% identical, or 100% identical to SEQ ID NO: 2.

In other embodiments, the recombinant polynucleotide encoding the galactose transporter protein is stably integrated into the genome of the isolated bacterial cell. Methods of stably integrating a recombinant polynucleotide into the genome of a bacterial cell are well known in the art and include, without limitation, homologous recombination.

In addition to the galactose transporter protein, isolated bacterial cells of the present disclosure may also contain at least one additional recombinant polynucleotide encoding a sugar transport protein, where expression of the sugar transporter protein results in transport of sugar into the photoautotrophic bacterial cell. Sugar transporter proteins are well known in the art and include, without limitation, proteins that transport hexose sugars, such as galactose, fructose, mannose, etc.; proteins that transport disaccharide sugars, such as sucrose, lactose, lactulose, maltose, trehalose, cellobiose, etc.; and proteins that transport pentose sugars, such as xylose, arabinose, ribose, ribulose, xylulose, etc.

Other aspects of the present disclosure relate to improved characteristics resulting from the expression of a recombinant polynucleotide encoding a galactose transporter protein of the present disclosure. These improved characteristics include, without limitation, increased growth on glucose under dark or diurnal conditions. These characteristics are improved when compared to a corresponding photoautotrophic bacterial cell of the same species that does not contain (i.e., lacks) the recombinant polynucleotide encoding the galactose transporter protein. Thus, in certain embodiments, an isolated bacterial cell of the present disclosure has increased growth, cell density, and/or biomass production on glucose under dark or diurnal conditions compared to a corresponding photoautotrophic bacterial cell lacking a recombinant polynucleotide encoding a galactose transporter protein of the present disclosure. In other embodiments, the growth, cell density, and/or biomass production of the isolated bacterial cells on glucose under dark or diurnal conditions is increased by at least 5%, at least 10%, at least 15%, at least 20%, at least 25%, at least 30%, at least 35%, at least 36%, at least 37%, at least 38%, at least 39%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 125%, at least 130%, at least 140%, at least 150%, at least 175%, at least 200%, at least 225%, at least 250%, at least 275%, at least 300%, at least 350%, at least 400%, at least 450%, at least 500%, any percentage, in whole integers between 5% and 500% (e.g., 6%, 7%, 8%, etc.), or more compared to a corresponding photoautotrophic bacterial cell lacking a recombinant polynucleotide encoding a galactose transporter protein of the present disclosure.

Disaccharide Transporter Proteins

In other embodiments, isolated bacterial cells of a photoautotrophic species of the present disclosure contain a recombinant polynucleotide encoding a disaccharide transporter protein, such as a sucrose transporter protein, where expression of the disaccharide sugar transporter protein results in transport of a disaccharide sugar into the bacterial cell to increase growth of the bacterial cell on the disaccharide sugar under dark or diurnal conditions as compared to a corresponding photoautotrophic bacterial cell lacking the recombinant polynucleotide. In some embodiments, the bacterial cell also contains the proteins necessary to convert the disaccharide sugar into its corresponding monosaccharides. Proteins necessary for converting a disaccharide sugar, such as sucrose, into its corresponding monosaccharides are well known in the art.

The disaccharide transporter protein may be a sucrose transporter protein, a lactose transporter protein, a lactulose transporter protein, a maltose transporter protein, a trehalose transporter protein, or a cellobiose transporter protein.

Sucrose is a natural metabolite of cyanobacteria, such as S. elongatus, and can be synthesized in response to osmotic pressure (Ducat D C et al., (2012) Appl Environ Microbiol 78(8):2660-2668; and Suzuki E et al, (2010) Appl Environ Microbiol 76(10):3153-3159). Accordingly, in certain preferred embodiments, the disaccharide transporter protein is a sucrose transporter protein. The sucrose transporter protein may be an E. coli CscB sucrose transporter protein, a B. subtilis SacP transporter protein, a Brassica napus Sut1 transporter protein, a Juglans regia Sut1 transporter protein, an Arabidopsis thaliana Suc6 transporter protein, an Arabidopsis thaliana SUT4 transporter protein, a Drosophila melanogaster Slc45-1 transporter protein, a Dickeya dadantii ScrA transporter protein, and homologs thereof. In certain preferred embodiments, the sucrose transporter protein is an E. coli CscB sucrose transporter protein, or homologs thereof. In other preferred embodiments, the sucrose transporter protein is an E. coli CscB sucrose transporter protein having an amino acid sequence that is at least at least 70%, at least 75%, at least 80%, at least 85%, at least 90%, at least 91%, at least 92%, at least 93%, at least 94%, at least 95%, at least 96%, at least 97%, at least 98%, or at least 99% identical, or 100% identical to SEQ ID NO: 14.

In other embodiments, the recombinant polynucleotide encoding the disaccharide transporter protein is stably integrated into the genome of the isolated bacterial cell. Methods of stably integrating a recombinant polynucleotide into the genome of a bacterial cell are well known in the art and include, without limitation, homologous recombination.

In addition to the disaccharide transporter protein, isolated bacterial cells of the present disclosure may also contain at least one additional recombinant polynucleotide encoding a sugar transport protein, where expression of the sugar transporter protein results in transport of sugar into the photoautotrophic bacterial cell. Sugar transporter proteins are well known in the art and include, without limitation, proteins that transport hexose sugars, such as galactose, fructose, mannose, etc.; proteins that transport disaccharide sugars, such as sucrose, lactose, lactulose, maltose, trehalose, cellobiose, etc.; and proteins that transport pentose sugars, such as xylose, arabinose, ribose, ribulose, xylulose, etc.

Other aspects of the present disclosure relate to improved characteristics resulting from the expression of a recombinant polynucleotide encoding a disaccharide transporter protein of the present disclosure. These improved characteristics include, without limitation, increased growth on a disaccharide, such as sucrose, under dark or diurnal conditions. These characteristics are improved when compared to a corresponding photoautotrophic bacterial cell of the same species that does not contain (i.e., lacks) the recombinant polynucleotide encoding the disaccharide transporter protein. Thus, in certain embodiments, an isolated bacterial cell of the present disclosure has increased growth, cell density, and/or biomass production on a disaccharide, such as sucrose, under dark or diurnal conditions compared to a corresponding photoautotrophic bacterial cell lacking a recombinant polynucleotide encoding a disaccharide transporter protein of the present disclosure. In other embodiments, the growth, cell density, and/or biomass production of the isolated bacterial cells on a disaccharide, such as sucrose, under dark or diurnal conditions is increased by at least 5%, at least 10%, at least 15%, at least 20%, at least 25%, at least 30%, at least 35%, at least 36%, at least 37%, at least 38%, at least 39%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 125%, at least 130%, at least 140%, at least 150%, at least 175%, at least 200%, at least 225%, at least 250%, at least 275%, at least 300%, at least 350%, at least 400%, at least 450%, at least 500%, any percentage, in whole integers between 5% and 500% (e.g., 6%, 7%, 8%, etc.), or more compared to a corresponding photoautotrophic bacterial cell lacking a recombinant polynucleotide encoding a disaccharide transporter protein of the present disclosure.

Xylose Transporter Proteins

Xylose is the major part of abundantly available hemicellulosic biomass, and may provide an inexpensive renewable feedstock for microbial production of a commodity chemical, such as biofuels (Steen E J et al., (2010) Nature 463(7280):559-562). However, xylose is not a known metabolite of photoautotrophic bacterial cells, such as the cyanobacterium S. elongatus.

Accordingly, in further embodiments, isolated bacterial cells of a photoautotrophic species of the present disclosure contain a recombinant polynucleotide encoding a xylose transporter protein, where expression of the xylose transporter protein results in transport of xylose into the bacterial cell to increase growth of the bacterial cell on xylose under dark or diurnal conditions as compared to a corresponding photoautotrophic bacterial cell lacking the recombinant polynucleotide.

The xylose transporter protein may an E. coli XylE xylose transporter protein, an E. coli xylF/xylG/xylH ABC xylose transporter protein, a Candida intermedia Gxf1 transporter protein, a Pichia stipitis Sut1 transporter protein, an A. thaliana At5g59250transporter protein, and homologs thereof. In certain preferred embodiments, the xylose transporter protein is an E. coli XylE xylose transporter protein, or homologs thereof. In other preferred embodiments, the xylose transporter protein is an E. coli XylE xylose transporter protein having an amino acid sequence that is at least at least 70%, at least 75%, at least 80%, at least 85%, at least 90%, at least 91%, at least 92%, at least 93%, at least 94%, at least 95%, at least 96%, at least 97%, at least 98%, or at least 99% identical, or 100% identical to SEQ ID NO: 8.

In other embodiments, the recombinant polynucleotide encoding the xylose transporter protein is stably integrated into the genome of the isolated bacterial cell. Methods of stably integrating a recombinant polynucleotide into the genome of a bacterial cell are well known in the art and include, without limitation, homologous recombination.

In addition to the xylose e transporter protein, isolated bacterial cells of the present disclosure may also contain at least one additional recombinant polynucleotide encoding a sugar transport protein, where expression of the sugar transporter protein results in transport of sugar into the photoautotrophic bacterial cell. Sugar transporter proteins are well known in the art and include, without limitation, proteins that transport hexose sugars, such as galactose, fructose, mannose, etc.; proteins that transport disaccharide sugars, such as sucrose, lactose, lactulose, maltose, trehalose, cellobiose, etc.; and proteins that transport pentose sugars, such as xylose, arabinose, ribose, ribulose, xylulose, etc.

Other aspects of the present disclosure relate to improved characteristics resulting from the expression of a recombinant polynucleotide encoding a xylose transporter protein of the present disclosure. These improved characteristics include, without limitation, increased growth on xylose under dark or diurnal conditions. These characteristics are improved when compared to a corresponding photoautotrophic bacterial cell of the same species that does not contain (i.e., lacks) the recombinant polynucleotide encoding the xylose transporter protein. Thus, in certain embodiments, an isolated bacterial cell of the present disclosure has increased growth, cell density, and/or biomass production on xylose under dark or diurnal conditions compared to a corresponding photoautotrophic bacterial cell lacking a recombinant polynucleotide encoding a xylose transporter protein of the present disclosure. In other embodiments, the growth, cell density, and/or biomass production of the isolated bacterial cells on xylose under dark or diurnal conditions is increased by at least 5%, at least 10%, at least 15%, at least 20%, at least 25%, at least 30%, at least 35%, at least 36%, at least 37%, at least 38%, at least 39%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 125%, at least 130%, at least 140%, at least 150%, at least 175%, at least 200%, at least 225%, at least 250%, at least 275%, at least 300%, at least 350%, at least 400%, at least 450%, at least 500%, any percentage, in whole integers between 5% and 500% (e.g., 6%, 7%, 8%, etc.), or more compared to a corresponding photoautotrophic bacterial cell lacking a recombinant polynucleotide encoding a disaccharide transporter protein of the present disclosure.

Additional Protein Components

In further embodiments, isolated bacterial cells of a photoautotrophic species of the present disclosure containing a recombinant polynucleotide encoding a sugar transporter protein may further contain at least one additional recombinant polynucleotide encoding at least one downstream metabolic enzyme that facilitates incorporation of the disclosed sugars into the central metabolism of the isolated bacterial cells. The at least one additional recombinant polynucleotide encoding the at least one downstream metabolic enzyme may be stably integrated into the genome of the isolated bacterial cell. Methods of stably integrating a recombinant polynucleotide into the genome of a bacterial cell are well known in the art and include, without limitation, homologous recombination.

In certain embodiments, the at least one downstream metabolic enzyme facilitates incorporation of a hexose sugar of the present disclosure into the central metabolism of the isolated bacterial cells of the present disclosure containing a recombinant polynucleotide encoding a hexose sugar transporter protein. Examples of suitable downstream metabolic galactose enzymes include, without limitation, galactose-1-epimerase, galactokinase, galactose-1-phosphate uridyltransferase, and UDP-glucose-4-epimerase. Examples of suitable downstream metabolic glucose enzymes include, without limitation, glucokinase. Examples of suitable downstream metabolic fructose enzymes include, without limitation, manno(fructo)kinase and fructokinase. Examples of suitable downstream metabolic mannose enzymes include, without limitation, manno(fructo)kinase, and Mannose-6-phosphate isomerase.

In certain embodiments, the at least one downstream metabolic enzyme facilitates incorporation of a disaccharide sugar of the present disclosure into the central metabolism of isolated bacterial cells of the present disclosure containing a recombinant polynucleotide encoding a disaccharide transporter protein. Examples of suitable downstream metabolic lactose enzymes include, without limitation, β-galactosidase. Examples of suitable downstream metabolic maltose enzymes include, without limitation, maltose phosphorylase and β-phosphoglucomutase. Examples of suitable downstream metabolic terehalose enzymes include, without limitation, treA from B. subtilis, treP and pgmB from L. lactis. Examples of suitable downstream metabolic cellobiose enzymes include, without limitation, Cel1a, Cel3a BGLI or BGLII from Hypocrea jecorina.

In certain preferred embodiments, the at least one downstream metabolic enzyme facilitates incorporation of sucrose into the central metabolism of isolated bacterial cells of the present disclosure containing a recombinant polynucleotide encoding a sucrose transporter protein. Examples of suitable downstream metabolic sucrose enzymes include, without limitation, fructokinase, sucrase-6-phosphate hydrolase and invertase. Examples of suitable fructokinases include, without limitation, a CscK protein encoded by the E. coli cscK gene, a Lycopersicon esculentum Frk1 fructokinase protein, a Lycopersicon esculentum Frk2 fructokinase protein, a H. sapiens KHK fructokinase protein, an A. thaliana FLN-1 fructokinase protein, an A. thaliana and FLN-2 fructokinase protein, a Yersinia pestis biovar Microtus str. 91001 NagC1 fructokinase protein, a Yersinia pseudotuberculosis YajF fructokinase protein, and a Natronomonas pharaonis Suk fructokinase protein, and homologs thereof. In certain preferred embodiments, the fructokinase is a CscK protein encoded by the E. coli cscK gene, or homologs thereof. In other preferred embodiments, the fructokinase is a CscK protein having an amino acid sequence that is at least at least 70%, at least 75%, at least 80%, at least 85%, at least 90%, at least 91%, at least 92%, at least 93%, at least 94%, at least 95%, at least 96%, at least 97%, at least 98%, or at least 99% identical, or 100% identical to SEQ ID NO: 16.

In certain embodiments, the at least one downstream metabolic enzyme facilitates incorporation of a pentose sugar of the present disclosure into the central metabolism of the isolated bacterial cells. Examples of suitable downstream metabolic arabinose enzymes include, without limitation, L-arabinose isomerase, L-ribulokinase, and L-ribulose-5-phosphate-4-epimerase. Examples of suitable downstream metabolic ribose enzymes include, without limitation, RbsK from E. coli.

In certain preferred embodiments, the at least one downstream metabolic enzyme facilitates incorporation of xylose into the central metabolism of isolated bacterial cells of the present disclosure containing a recombinant polynucleotide encoding a xylose transporter protein. Examples of suitable downstream metabolic xylose enzymes include, without limitation, xylose isomerase and xylulokinase. The isolated bacterial cells of the present disclosure containing a recombinant polynucleotide encoding a xylose transporter protein may further contain an additional recombinant polynucleotide encoding a xylose isomerase and/or an additional recombinant polynucleotide encoding a xylulokinase. Alternatively, isolated bacterial cells of the present disclosure containing a recombinant polynucleotide encoding a xylose transporter protein may further contain an additional recombinant polynucleotide encoding both a xylose isomerase and a xylulokinase. Preferably, isolated bacterial cells of the present disclosure that can grow on xylose contain a recombinant polynucleotide encoding a xylose transporter protein, a xylose isomerase and a xylulokinase. In certain embodiments, the xylose isomerase is a XylA protein encoded by the E. coli xylA gene, an A. thaliana AT5G57655 xylose isomerase, an Aspergillus niger XyrA xylose isomerase, a Hypocrea jecorina Xyl1 xylose isomerase, and homologs thereof. In certain preferred embodiments, the xylose isomerase is a XylA protein encoded by the E. coli xylA gene, or homologs thereof. In other preferred embodiments, the xylose isomerase is an E. coli XylA protein having an amino acid sequence that is at least at least 70%, at least 75%, at least 80%, at least 85%, at least 90%, at least 91%, at least 92%, at least 93%, at least 94%, at least 95%, at least 96%, at least 97%, at least 98%, or at least 99% identical, or 100% identical to SEQ ID NO: 10.

In other embodiments, the xylulokinase is a XylB protein encoded by the E. coli xylB gene, an Arabidopsis thaliana XK-1 xylulokinase, an Arabidopsis thaliana XK-2 xylulokinase, an E. coli LynK xylulokinase, a Streptomyces coelicolor SCO1170 xylulokinase, a Pseudomonas aeruginosa MtlY xylulokinase, a Yersinia pseudotuberculosis SgbK xylulokinase, an E. coli AtlK xylulokinase, and homologs thereof. In certain preferred embodiments, the xylulokinase is a XylB protein encoded by the E. coli xylB gene, or homologs thereof. In other preferred embodiments, the xylulokinase is an E. coli XylB protein having an amino acid sequence that is at least at least 70%, at least 75%, at least 80%, at least 85%, at least 90%, at least 91%, at least 92%, at least 93%, at least 94%, at least 95%, at least 96%, at least 97%, at least 98%, or at least 99% identical, or 100% identical to SEQ ID NO: 12.

Additionally, isolated bacterial cells of the present disclosure may be engineered to express combinations of the disclosed sugar transporter proteins and downstream metabolic enzymes. Non-limiting examples of such combinations include glucose and fructose transport with metabolic enzymes; glucose and xylose transport with metabolic enzymes; glucose, xylose, and arabinose transport with metabolic enzymes; glucose, fructose, xylose, and arabinose transport with metabolic enzymes; glucose, fructose, mannose, galactose, xylose, and arabinose transport with metabolic enzymes; and sucrose and lactose transport with metabolic enzymes.

### Polynucleotide Constructs Encoding Sugar Transporter Proteins

Other aspects of the present disclosure relate to polynucleotide constructs containing polynucleotide sequences encoding one or more sugar transporter proteins and/or downstream metabolic enzymes. The polynucleotides of the present disclosure may be operably linked to promoters and optional control sequences such that the subject proteins are expressed in an isolated bacterial cell of the present disclosure cultured under suitable conditions. The promoters and control sequences may be specific for the photoautotrophic species of each isolated bacterial cell of the present disclosure. In some embodiments, expression vectors contain the polynucleotide constructs. Methods for designing and making polynucleotide constructs and expression vectors are well known to those skilled in the art.

As used herein, the terms “polynucleotide sequence,” “sequence of polynucleotides,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. Thus, these terms include known types of polynucleotide sequence modifications, for example, substitution of one or more of the naturally occurring nucleotides with an analog; internucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970).

Sequences of polynucleotides encoding the subject proteins are prepared by any suitable method known to those of ordinary skill in the art, including, for example, direct chemical synthesis or cloning. For direct chemical synthesis, formation of a polymer of polynucleotides typically involves sequential addition of 3′-blocked and 5′-blocked nucleotide monomers to the terminal 5′-hydroxyl group of a growing nucleotide chain, where each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired polynucleotide sequence from the gel via techniques known to those of ordinary skill in the art, such as utilization of polymerase chain reactions (PCR; e.g., U.S. Pat. No. 4,683,195).

Each polynucleotide sequence encoding the desired subject protein can be incorporated into an expression vector. “Expression vector” or “vector” refers to a compound and/or composition that transduces, transforms, or infects an isolated bacterial cell of the present disclosure, thereby causing the cell to express polynucleotides and/or proteins other than those endogenous to the cell, or in a manner not naturally occurring in the cell. An expression vector contains a sequence of polynucleotides (ordinarily RNA or DNA) to be expressed by the isolated bacterial cell. Optionally, the expression vector also contains materials to aid in achieving entry of the polynucleotide into the isolated bacterial cells, such as a virus, liposome, protein coating, or the like. The expression vectors contemplated for use in the present disclosure include those into which a polynucleotide sequence can be inserted, along with any preferred or required operational elements. Further, the expression vector can be transferred into an isolated bacterial cell and replicated therein. Once transferred or transformed into a suitable isolated bacterial cell, the vector may replicate and function independently of the host genome, or may, in some instances, integrate into the genome itself. Examples of expression vectors include, without limitation, a plasmid, a phage particle, or simply a potential genomic insert. Preferred expression vectors are plasmids, particularly those with restriction sites that have been well-documented and that contain the operational elements preferred or required for transcription of the polynucleotide sequence. Such plasmids, as well as other expression vectors, are well known to those of ordinary skill in the art.

Incorporation of the individual polynucleotide sequences may be accomplished through known methods that include, for example, the use of restriction enzymes (such as BamHI, EcoRI, Hhal, Xhol, Xmal, and so forth) to cleave specific sites in the expression vector, e.g., plasmid. The restriction enzyme produces single-stranded ends that may be annealed to a polynucleotide sequence having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired polynucleotide sequence are often cleaved with the same restriction enzyme, thereby assuring that the ends of the expression vector and the ends of the polynucleotide sequence are complementary to each other. In addition, DNA linkers may be used to facilitate linking of polynucleotide sequences into an expression vector.

A series of individual polynucleotide sequences can also be combined by utilizing methods that are known to those having ordinary skill in the art (e.g., U.S. Pat. No. 4,683,195). For example, each of the desired polynucleotide sequences can be initially generated in a separate PCR. Thereafter, specific primers are designed such that the ends of the PCR products contain complementary sequences. When the PCR products are mixed, denatured, and reannealed, the strands may have matching sequences at their 3′ end overlap and can act as primers for each other. Extension of this overlap by DNA polymerase produces a molecule in which the original sequences are “spliced” together. In this way, a series of individual polynucleotide sequences may be “spliced” together and subsequently transduced into an isolated bacterial cell simultaneously. Thus, expression of each of the plurality of polynucleotide sequences is affected.

Individual polynucleotide sequences, or “spliced” polynucleotide sequences, are then incorporated into an expression vector. The present disclosure is not limited with respect to the process by which the polynucleotide sequence is incorporated into the expression vector. Those of ordinary skill in the art are familiar with the necessary steps for incorporating a polynucleotide sequence into an expression vector. A typical expression vector contains the desired polynucleotide sequence preceded by one or more regulatory regions, along with a ribosome binding site, e.g., a nucleotide sequence that is 3-9 nucleotides in length and located 3-11 nucleotides upstream of the initiation codon in E. coli (see Shine et al. (1975), Nature 254:34 and Steitz, Biological Regulation and Development: Gene Expression (ed. R. F. Goldberger), vol. 1, p. 349, 1979, Plenum Publishing, NY).

Regulatory regions include, for example, those regions that contain a promoter and an operator. A promoter is operably linked to the desired polynucleotide sequence, thereby initiating transcription of the polynucleotide sequence via an RNA polymerase enzyme. An operator is a sequence of polynucleotides adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. Examples include lactose promoters (Lad repressor protein changes conformation when contacted with lactose, thereby preventing the Lad repressor protein from binding to the operator) and tryptophan promoters (when complexed with tryptophan, TrpR repressor protein has a conformation that binds the operator; in the absence of tryptophan, the TrpR repressor protein has a conformation that does not bind to the operator). Another example is the tac promoter (see deBoer et al. (1983) Proc Natl Acad Sci USA, 80:21-25). As will be appreciated by those of ordinary skill in the art, these and other expression vectors may be used in the present disclosure, and the present disclosure is not limited in this respect. In certain embodiments, the promoter is the lac-regulatable P_{trc} promoter.

Although any suitable expression vector may be used to incorporate the desired sequences, readily-available expression vectors include, without limitation, plasmids, such as pAM2991, pSClOl, pBR322, pBBRlMCS-3, pUR, pEX, pMRlOO, pCR4, pBAD24, pUC19, and bacteriophages, such as M13 phage and λ phage. Of course, such expression vectors may only be suitable for particular bacterial cells of a photoautotrophic species. One of ordinary skill in the art, however, can readily determine through routine experimentation whether any particular expression vector is suited for any given isolated bacterial cell. For example, the expression vector can be introduced into the isolated bacterial cell, which is then monitored for viability and expression of the sequences contained in the vector. In addition, reference may be made to the relevant texts and literature, which describe expression vectors and their suitability to any particular isolated bacterial cell.

### Methods of Producing and Culturing Isolated Bacterial Cells

The expression vectors of the present disclosure are introduced or transferred into an isolated bacterial cell of a photoautotrophic species of the present disclosure. Such methods for transferring the expression vectors into isolated bacterial cells are well known to those of ordinary skill in the art. For example, one method for transforming bacterial cells with an expression vector involves a calcium chloride treatment where the expression vector is introduced via a calcium precipitate. Other salts, e.g., calcium phosphate, may also be used following a similar procedure. In addition, electroporation (i.e., the application of a current to increase the permeability of cells to polynucleotide sequences) may be used to transfect the isolated bacterial cell. Also, microinjection of the polynucleotide sequences provides the ability to transfect isolated bacterial cells. Other means, such as lipid complexes, liposomes, and dendrimers, may also be employed. Those of ordinary skill in the art can transfect an isolated bacterial cell with a desired sequence using these or other methods.

For identifying a transformed bacterial cell, a variety of methods are available. For example, a culture of potentially transformed bacterial cells may be separated, using a suitable dilution, into individual cells and thereafter individually grown and tested for expression of the desired polynucleotide sequence. In addition, when plasmids are used, an often-used practice involves the selection of cells based upon antimicrobial resistance that has been conferred by genes intentionally contained within the expression vector, such as the amp, kan, gpt, neo, and hyg genes.

Once the isolated bacterial cell has been transformed with the expression vector, the isolated bacterial cell is allowed to grow. Methods of the present disclosure include culturing the isolated bacterial cell such that recombinant polynucleotides in the cell are expressed. For bacterial cells of a photoautotrophic species, this process entails culturing the cells in a suitable medium. Typically cells can be grown at temperatures from about 25° C. to about 35° C. in appropriate media. Preferred growth media of the present disclosure are common commercially prepared media such as BG-11 medium. Other defined or synthetic growth media may also be used, and the appropriate medium for growth of the particular bacterial cell of a photoautotrophic species will be known by someone skilled in the art of microbiology or bacteriology.

According to some aspects of the present disclosure, the culture media may contain a carbon source for the isolated bacterial cell. Such a “carbon source” generally refers to a substrate or compound suitable to be used as a source of carbon for bacterial cell growth. Carbon sources can be in various forms, including, but not limited to polymers, carbohydrates, acids, alcohols, aldehydes, ketones, amino acids, peptides, etc. These include, for example, various monosaccharides, such as glucose, galactose, xylose, and arabinose; disaccharides, such as sucrose and lactose; oligosaccharides; polysaccharides; biomass polymers, such as cellulose and hemicellulose; saturated or unsaturated fatty acids; succinate; lactate; acetate; ethanol; etc.; or mixtures thereof. Multiple biomass polymers may be generated by treating plant biomass with ionic liquid. This treated biomass may then be added to a culture so that the culture contains more than one biomass polymer. In preferred embodiments of the present disclosure, the carbon source is a sugar substrate such as a monosaccharide or a disaccharide.

In addition to an appropriate carbon source, fermentation media for the production of a biofuel may contain suitable minerals, salts, cofactors, buffers and other components, known to those skilled in the art, suitable for the growth of the cultures and promotion of the enzymatic pathways necessary for production of fatty acid-derived molecules. Reactions may be performed under aerobic or anaerobic conditions where aerobic, anoxic, or anaerobic conditions are preferred based on the requirements of the microorganism. As the isolated bacterial cell grows and/or multiplies, the proteins necessary for producing a commodity chemical such as a biofuel are expressed.

### Embodiments Relating to Increasing Growth, Cell Density, and Biomass Production of Bacterial Cells

As disclosed herein, expression of a sugar transporter protein results in increased growth on a sugar substrate, increased cell density, and increased biomass production of bacterial cells of a photoautotrophic species under dark or diurnal conditions compared to wild-type or untransformed photoautotrophic bacterial cells of the same species.

Accordingly, certain embodiments of the present disclosure relate to methods of increasing bacterial growth, by providing a bacterial cell of a photoautotrophic species containing a recombinant polynucleotide encoding a sugar transporter protein; and culturing the bacterial cell with a sugar substrate under conditions whereby the recombinant polynucleotide is expressed, where expression of the recombinant polynucleotide results in transport of the sugar substrate into the bacterial cell to increase cell growth on sugar under dark or diurnal conditions as compared to cell growth of a corresponding photoautotrophic bacterial cell of the same species lacking the recombinant polynucleotide encoding the sugar transporter protein. In other embodiments, growth of the bacterial cells expressing a sugar transporter on a sugar substrate under dark or diurnal conditions is increased by at least 5%, at least 10%, at least 15%, at least 20%, at least 25%, at least 30%, at least 35%, at least 36%, at least 37%, at least 38%, at least 39%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 125%, at least 130%, at least 140%, at least 150%, at least 175%, at least 200%, at least 225%, at least 250%, at least 275%, at least 300%, at least 350%, at least 400%, at least 450%, at least 500%, any percentage, in whole integers between 5% and 500% (e.g., 6%, 7%, 8%, etc.), or more as compared to a corresponding photoautotrophic bacterial cell of the same species that lacks the recombinant polynucleotide encoding the sugar transporter protein.

Other embodiments relate to methods of increasing bacterial cell density under dark or diurnal conditions, by providing a bacterial cell of a photoautotrophic species containing a recombinant polynucleotide encoding a sugar transporter protein; and culturing the bacterial cell with a sugar substrate under conditions whereby the recombinant polynucleotide is expressed, where expression of the recombinant polynucleotide results in transport of the sugar substrate into the bacterial cell to increase cell density under dark or diurnal conditions as compared to a corresponding photoautotrophic bacterial cell of the same species that lacks the recombinant polynucleotide encoding the sugar transporter protein. In other embodiments, cell density of the bacterial cells expressing a sugar transporter on a sugar substrate under diurnal conditions is increased by at least 5%, at least 10%, at least 15%, at least 20%, at least 25%, at least 30%, at least 35%, at least 36%, at least 37%, at least 38%, at least 39%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 125%, at least 130%, at least 140%, at least 150%, at least 175%, at least 200%, at least 225%, at least 250%, at least 275%, at least 300%, at least 350%, at least 400%, at least 450%, at least 500%, any percentage, in whole integers between 5% and 500% (e.g., 6%, 7%, 8%, etc.), or more as compared to a corresponding photoautotrophic bacterial cell of the same species that lacks the recombinant polynucleotide encoding the sugar transporter protein.

Further embodiments relate to methods of increasing bacterial biomass production under dark or diurnal conditions, by providing a bacterial cell of a photoautotrophic species containing a recombinant polynucleotide encoding a sugar transporter protein; and culturing the photoautotrophic bacterial cell with a sugar substrate under conditions whereby the recombinant polynucleotide is expressed, where expression of the recombinant polynucleotide results in transport of the sugar substrate into the bacterial cell to increase biomass production under dark or diurnal conditions as compared to a corresponding photoautotrophic bacterial cell of the same species that lacks the recombinant polynucleotide encoding the sugar transporter protein. In other embodiments, biomass production of the bacterial cells expressing a sugar transporter on a sugar substrate under diurnal conditions is increased by at least 5%, at least 10%, at least 15%, at least 20%, at least 25%, at least 30%, at least 35%, at least 36%, at least 37%, at least 38%, at least 39%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 125%, at least 130%, at least 140%, at least 150%, at least 175%, at least 200%, at least 225%, at least 250%, at least 275%, at least 300%, at least 350%, at least 400%, at least 450%, at least 500%, any percentage, in whole integers between 5% and 500% (e.g., 6%, 7%, 8%, etc.), or more as compared to a corresponding photoautotrophic bacterial cell of the same species that lacks the recombinant polynucleotide encoding the sugar transporter protein.

In other embodiments, the recombinant polynucleotide encodes a sugar transporter protein selected from a hexose sugar transporter, a galactose transporter, a glucose transporter, a fructose transporter, a mannose transporter, a Major Facilitator Superfamily (MFS) transporter, a Phosphotransferase System (PTS) transporter, a pentose transporter, a xylose transporter, an arabinose transporter, a ribose transporter, a ribulose transporter, a xylulose transporter, and a homolog thereof. In other embodiments, the recombinant polynucleotide encodes a disaccharide sugar transporter protein selected from a sucrose transporter, a lactose transporter, a lactulose transporter, a maltose transporter, a trehalose transporter, a cellobiose transporter, and a homolog thereof. In embodiments where the bacterial cell contains a disaccharide sugar transporter protein, the bacterial cell may further contain the proteins necessary to convert the disaccharide sugar into its corresponding monosaccharides.

In certain embodiments, the recombinant polynucleotide encodes a galactose transporter protein. In yet other embodiments, the galactose transporter protein is selected from a bacterial galP transporter, an E. coli galP transporter, a eukaryotic galP transporter, a fungal galP transporter, a mammalian galP transporter, a bacterial MFS transporter, a eukaryotic MFS, a fungal MFS, a mammalian MFS, a bacterial PTS transporter, a eukaryotic PTS, a fungal PTS, a mammalian PTS, and a homolog thereof. Preferably, the galactose transporter protein transports glucose into the bacterial cell.

In other embodiments, the recombinant polynucleotide encodes a disaccharide transporter protein. In certain embodiments, the disaccharide transporter protein is selected from a sucrose transporter protein, a fructose transporter protein, a lactose transporter protein, a lactulose transporter protein, a maltose transporter protein, a trehalose transporter protein, a cellobiose transporter protein, and a homolog thereof. Preferably, the disaccharide transporter protein is a sucrose transporter protein. More preferably, the sucrose transporter protein is an E. coli CscB sucrose transporter protein. In further embodiments, a bacterial cell of the present disclosure containing a recombinant polynucleotide encoding a sucrose transporter protein further contains at least one additional recombinant polynucleotide encoding a fructokinase protein of the present disclosure.

In other embodiments, the recombinant polynucleotide encodes a xylose transporter protein. In certain preferred embodiments, the xylose transporter protein is an E. coli XylE xylose transporter protein. In further embodiments, a bacterial cell of the present disclosure containing a recombinant polynucleotide encoding a xylose transporter protein further contains at least one additional recombinant polynucleotide encoding a xylose isomerase and/or xylulokinase protein of the present disclosure. In certain embodiments, the xylose transporter protein, xylose isomerase, and/or xylulokinase protein are encoded by a single recombinant polynucleotide. Preferably the xylose isomerase is an E. coli XylA xylose isomerase, and the xylulokinase is an E. coli XylB xylulokinase.

In further embodiments, the recombinant polynucleotide is stably integrated into the genome of the bacterial cell. Methods of stably integrating a recombinant polynucleotide into the genome of the bacterial cell are well known in the art and include, without limitation, homologous recombination.

In addition to the sugar transporter protein, the bacterial cells of the present disclosure may also contain at least one additional recombinant polynucleotide encoding a second sugar transport protein, a third sugar transport protein, a fourth sugar transport protein, a fifth sugar transport protein, or more sugar transport proteins, where expression of each sugar transporter protein results in transport of a second, third, fourth, fifth, or more sugars into the bacterial cell. The second, third, fourth, fifth, or more sugar transporter proteins may transport the same type of sugar as the first sugar transporter, or may transport a sugar that is distinct from that transported by the first sugar transporter. The second, third, fourth, fifth, or more sugar transporter proteins may be any of the disclosed sugar transporters.

The methods of the present disclosure utilize bacterial cells of a photoautotrophic species that express a sugar transporter protein. These bacterial cells can utilize exogenous sugar substrate as a carbon source in order to grow during the dark phases of diurnal cycles. Moreover, utilization of an exogenous sugar substrate by the bacterial cells expressing a sugar transporter protein is compatible with and compliments photosynthesis performed by the bacterial cells. In this way, the bacterial cells can grow continuously 24 hours a day. Exogenous sugar substrate is provided as part of the step of culturing the bacterial cells, and may be provided in the culture medium.

Suitable sugar substrates that are added during the culturing of the bacterial cells of a photoautotrophic species include sugars that can be transported by the sugar transporter protein that is expressed by the bacterial cells. Examples of suitable sugars include, without limitation, hexoses, such as galactose, glucose, fructose, and mannose; and pentoses such as xylose, arabinose, ribose, ribulose, and xylulose. Additional examples of suitable sugars include disaccharide sugars, such as sucrose, lactose, lactulose, maltose, trehalose, and cellobiose.

Additionally, sugar substrates may also include, without limitation, plant biomass, lignocellulosic biomass, biomass polymers, lignocellulose, cellulose, hemicellulose, polysaccharides, or mixtures thereof. Sources of such compositions include, without limitation, grasses (e.g., switchgrass, Miscanthus), rice hulls, bagasse, cotton, jute, eucalyptus, hemp, flax, bamboo, sisal, abaca, straw, leaves, grass clippings, corn stover, corn cobs, distillers grains, legume plants, sorghum, sugar cane, sugar beet pulp, wood chips, sawdust, and biomass crops (e.g., Crambe). Moreover, sources of such substrates may be an unrefined plant feedstock (e.g., ionic liquid-treated plant biomass) or a refined biomass polymer (e.g., beechwood xylan or phosphoric acid swollen cellulose).

Accordingly, in certain embodiments, the bacterial cell of a photoautotrophic species is cultured with a sugar selected from a hexose, galactose, glucose, fructose, mannose, a pentose, xylose, arabinose, ribose, ribulose, and xylulose. In other embodiments, the bacterial cell is cultured with a sugar selected from a disaccharide sugar, sucrose, lactose, lactulose, maltose, trehalose, and cellobiose. In certain preferred embodiments, the bacterial cell is cultured with glucose.

Additionally, bacterial cells of a photoautotrophic species of the present disclosure may be mutated through random mutagenesis to generate a library of mutants that can be screened for bacterial mutants that can utilize a sugar substrate as a sole carbon source in the complete and extended absence of light. Methods of random mutagenesis and screening are well known in the art. Examples of methods of random mutagenesis include, without limitation, chemical mutagenesis and radiation mutagenesis.

### Methods of Producing Commodity Chemicals

Certain aspects of the present disclosure further relate to isolated bacterial cells of a photoautotrophic species that produce commodity chemicals and to methods of producing commodity chemicals. Commodity chemicals include, without limitation, any saleable or marketable chemical that can be produced either directly or as a by-product of the disclosed isolated bacterial cells. Examples of commodity chemicals include, without limitation, biofuels, polymers, specialty chemicals, and pharmaceutical intermediates. Biofuels include, without limitation, alcohols such as ethanol, propanol, isopropanol, acetone, butanol, isobutanol, 2-methyl-1-butanol, 3-methyl-1-butanol, phenylethanol, fatty alcohols, and isopentenol; aldehydes, such as acetylaldehyde, propionaldehyde, butryaldehyde, isobutyraldehyde, 2-methyl-1-butanal, 3-methyl-1-butanal, phenylacetaldehyde, and fatty aldehydes; hydrocarbons, such as alkanes, alkenes, isoprenoids, fatty acids, wax esters, and ethyl esters; and inorganic fuels such as hydrogen. Polymers include, without limitation, 2,3-butanediol, 1,3-propandiol, 1,4-butanediol, polyhydroxyalkanoate, polyhydroxybutyrate, and isoprene. Specialty chemicals include, without limitation, carotenoids, such as lycopene, β-carotene, etc. Pharmaceutical intermediates include, without limitation, polyketides, statins, omega-3 fatty acids, isoprenoids, steroids, and erythromycin (antibiotic). Further examples of commodity chemicals include, without limitation, lactate, succinate, glutamate, citrate, malate, 3-hydroxypropionate, ascorbate, sorbitol, amino acids (leucine, valine, isoleucine, etc.), and hydroxybutyrate.

In some embodiments, an isolated bacterial cell of the present disclosure naturally produces any of the precursors for the production of the desired commodity chemical. These genes encoding the desired enzymes may be heterologous to the isolated bacterial cell, or these genes may be endogenous to the isolated bacterial cell but are operatively linked to heterologous promoters and/or control regions which result in higher expression of the gene(s) in the bacterial cell. For example, in certain embodiments, an isolated bacterial cell of the present disclosure may be further modified to overexpress metabolic genes involved in sugar digestion, including without limitation glycolytic, pentose phosphate, and tricarboxylic acid cycle genes.

In other embodiments, an isolated bacterial cell of the present disclosure does not naturally produce the desired commodity chemical, and thus contains heterologous polynucleotide constructs capable of expressing one or more genes necessary for producing the desired commodity chemical. Examples of such heterologous genes that allow bacterial cells to produce commodity chemicals are disclosed in PCT publication WO 2010/071581 and U.S. Patent Application Publication Nos. US 2010/0068776 and US 2011/0053216.

Additionally, isolated bacterial cells of the present disclosure may be engineered to produce ethanol by expressing, for example, pyruvate decarboxylase (pdc) and alcohol dehydrogenase (adhB) from Zymomonas mobilis (or homologues thereof). Isolated bacterial cells of the present disclosure may also be engineered to produce isobutyraldehyde by expressing, for example, 2-acetolactate synthase (alsS) from Bacillus subtilis, acetohydroxy acid isomeroreductase (ilvC) and dihydroxy acid dehydratase (ilvD) from Escherichia coli, and 2-ketoisovalerate decarboxylase (kivd) from Lactococcus lactis. Isolated bacterial cells of the present disclosure may further be engineered to produce isobutanol by expressing, for example, all genes responsible for isobutyraldehyde production along with alcohol dehydrogenase (yqhD) from Escherichia coli. Moreover, isolated bacterial cells of the present disclosure may be engineered to produce other higher order chain alcohols, such as 1-propanol, 1-butanol, 2-methyl-1-butanol, 3-methyl-1-butanol, and 2-phenylethanol, by expressing, for example, 2-ketoisovalerate decarboxlase (kivd) from Lactococcus lactis and alcohol dehydrogenase (yqhD) from Escherichia coli.

The present disclosure also provides for isolating a commodity chemical produced from the methods of the present disclosure. Isolating the commodity chemical involves separating at least part or all of the isolated bacterial cells, and parts thereof, from which the commodity chemical was produced, from the isolated commodity chemical. The isolated commodity chemical may be free or essentially free of impurities formed from at least part or all of the bacterial cells, and parts thereof. The isolated commodity chemical is essentially free of these impurities when the amount and properties of the impurities remaining do not interfere in the use of the commodity chemical.

Accordingly, in certain embodiments, an isolated bacterial cell of a photoautotrophic species containing a sugar transporter protein of the present disclosure further contains the proteins necessary for the bacterial cell to produce at least one commodity chemical. In certain embodiments, the isolated bacterial cell produces at least one commodity chemical.

Other aspects of the present disclosure relate to methods of producing at least one commodity chemical by providing a bacterial cell of a photoautotrophic species containing a recombinant polynucleotide encoding a sugar transporter protein; culturing the photoautotrophic bacterial cell with a sugar substrate under conditions whereby the recombinant polynucleotide is expressed and at least one commodity chemical is produced; and collecting the at least one commodity chemical, where expression of the recombinant polynucleotide results in transport of the sugar substrate into the bacterial cell. In certain embodiments, the bacterial cell contains the proteins necessary for the bacterial cell to produce the at least one commodity chemical. Exemplary sugar transporter proteins and sugar substrates are as described in previous sections. In embodiments where the bacterial cell expresses a disaccharide sugar transporter, the bacterial cell may further contain the proteins necessary to convert the disaccharide sugar into its corresponding monosaccharides. In other embodiments, the recombinant polynucleotide is stably integrated into the genome of the bacterial cell. In still other embodiments, the bacterial cell further contains at least one additional recombinant polynucleotide encoding a second sugar transport protein, a third sugar transport protein, a fourth sugar transport protein, a fifth sugar transport protein, or more sugar transport proteins, where expression of each sugar transporter protein results in transport of a second, third, fourth, fifth, or more sugar substrate into the bacterial cell. The second, third, fourth, fifth, or more sugar transporter proteins may transport the same type of sugar substrate as the first sugar transporter, or may transport a sugar substrate that is distinct from that transported by the first sugar transporter. The second, third, fourth, fifth, or more sugar transporter proteins may be any of the disclosed sugar transporters.

As disclosed herein, expression of a sugar transporter protein, such as a galactose transporter protein, a disaccharide transporter protein, or xylose transporter protein allows the bacterial cells to continually produce a commodity chemical under diurnal conditions. That is, the bacterial cells produce the commodity chemical during the day by utilizing photosynthesis and during the night (i.e., in the dark) by utilizing an exogenously provided sugar substrate as a carbon source. This in turn allows the bacterial cells to continually produce at least one commodity chemical 24 hours a day. Thus, in certain embodiments, the bacterial cell continually produces the at least one commodity chemical under diurnal conditions. Preferably, the at least one commodity chemical is continually produced 24 hours a day.

In other embodiments, the at least one produced commodity chemical is selected from a polymer, 2,3-butanediol, 1,3-propandiol, 1,4-butanediol, polyhydroxyalkanoate, polyhydroxybutyrate, isoprene, lactate, succinate, glutamate, citrate, malate, 3-hydroxypropionate, ascorbate, sorbitol, an amino acid, hydroxybutyrate, a carotenoid, lycopene, β-carotene, a pharmaceutical intermediate, a polyketide, a statin, an omega-3 fatty acid, an isoprenoid, a steroid, an antibiotic, erythromycin, a soprenoid, a steroid, erythromycin, a biofuel, and combinations thereof. In further embodiments, the produced commodity chemical is a biofuel selected from an alcohol, ethanol, propanol, isopropanol, acetone, butanol, isobutanol, 2-methyl-1-butanol, 3-methyl-1-butanol, phenylethanol, a fatty alcohol, isopentenol, an aldehyde, acetylaldehyde, propionaldehyde, butryaldehyde, isobutyraldehyde, 2-methyl-1-butanal, 3-methyl-1-butanal, phenylacetaldehyde, a fatty aldehyde, a hydrocarbon, an alkane, an alkene, an isoprenoids, a fatty acid, a wax ester, an ethyl ester, hydrogen, and combinations thereof.

### Supplemental Information

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989); Oligonucleotide Synthesis (Gait, ed., 1984); Animal Cell Culture (Freshney, ed., 1987); Handbook of Experimental Immunology (Weir & Blackwell, eds.); Gene Transfer Vectors for Mammalian Cells (Miller & Calos, eds., 1987); Current Protocols in Molecular Biology (Ausubel et al., eds., 1987); PCR: The Polymerase Chain Reaction, (Mullis et al., eds., 1994); Current Protocols in Immunology (Coligan et al., eds., 1991); The Immunoassay Handbook (Wild ed., Stockton Press NY, 1994); Bioconjugate Techniques (Hermanson, ed., Academic Press, 1996); and Methods of Immunological Analysis (Masseyeff, Albert, and Staines, eds., Weinheim: VCH Verlags gesellschaft mbH, 1993).

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. Thus, the term “homologous proteins” is defined to mean that the two proteins have similar amino acid sequences.

As used herein, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. Similarly, two polynucleotides (or a region of the polynucleotides) are substantially homologous when the nucleic acid sequences have at least about 30%, 40%, 50% 60%, 65%, 70%, 75%, 80%, 85%, 90%, 91%, 92%, 93%, 94%, 95%, 96%, 97%, 98%, or 99% identity . To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The percent identity between the two sequences is a function of the number of identical positions shared by the sequences, taking into account the number of gaps, and the length of each gap, which need to be introduced for optimal alignment of the two sequences. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. When comparing two sequences for identity, it is not necessary that the sequences be contiguous, but any gap would carry with it a penalty that would reduce the overall percent identity. For blastn, the default parameters are Gap opening penalty=5 and Gap extension penalty=2. For blastp, the default parameters are Gap opening penalty=11 and Gap extension penalty=1.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted using known algorithms (e.g., by the local homology algorithm of Smith and Waterman, Adv Appl Math, 2:482, 1981; by the homology alignment algorithm of Needleman and Wunsch, J Mol Biol, 48:443, 1970; by the search for similarity method of Pearson and Lipman, Proc Natl Acad Sci USA, 85:2444, 1988; by computerized implementations of these algorithms FASTDB (Intelligenetics), BLAST (National Center for Biomedical Information), GAP, BESTFIT, FASTA, and TFASTA in the Wisconsin Genetics Software Package (Genetics Computer Group, Madison, Wis.), or by manual alignment and visual inspection.

A preferred example of an algorithm that is suitable for determining percent sequence identity and sequence similarity is the FASTA algorithm (Pearson and Lipman, Proc Natl Acad Sci USA, 85:2444, 1988; and Pearson, Methods Enzymol, 266:227-258, 1996). Preferred parameters used in a FASTA alignment of DNA sequences to calculate percent identity are optimized, BL50 Matrix 15:-5, k-tuple=2; joining penalty=40, optimization=28; gap penalty-12, gap length penalty=-2; and width=16.

Another preferred example of algorithms suitable for determining percent sequence identity and sequence similarity are the BLAST and BLAST 2.0 algorithms (Altschul et al., Nuc Acids Res, 25:3389-3402, 1977; and Altschul et al., J Mol Biol, 215:403-410, 1990, respectively). BLAST and BLAST 2.0 are used, with the parameters described herein, to determine percent sequence identity for the nucleic acids and proteins of the disclosure. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (Henikoff and Henikoff, Proc Natl Acad Sci USA, 89:10915, 1989) alignments (B) of 50, expectation (E) of 10, M=5, N=-4, and a comparison of both strands.

The BLAST algorithm also performs a statistical analysis of the similarity between two sequences (See, e.g., Karlin and Altschul, Proc Natl Acad Sci USA, 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method (Feng and Doolittle, J Mol Evol, 35:351-360, 1987), employing a method similar to a published method (Higgins and Sharp, CABIOS 5:151-153, 1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc Acids Res, 12:387-395, 1984).

Another preferred example of an algorithm that is suitable for multiple DNA and amino acid sequence alignments is the CLUSTALW program (Thompson et al., Nucl Acids. Res, 22:4673-4680, 1994). ClustalW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff and Henikoff, Proc Natl Acad Sci USA, 89:10915-10919, 1992).

Polynucleotides of the disclosure further include polynucleotides that encode conservatively modified variants of the polypeptides encoded by the genes of Table 4 and the nucleic acid and amino acid sequences of SEQS ID NOS:1-16. “Conservatively modified variants” as used herein include individual mutations that result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups contain amino acids that are conservative substitutions for one another: 1. Alanine (A), Glycine (G); 2. Aspartic acid (D), Glutamic acid (E); 3. Asparagine (N), Glutamine (Q); 4. Arginine (R), Lysine (K); 5. Isoleucine (I), Leucine (L), Methionine (M), Valine (V); 6. Phenylalanine (F), Tyrosine (Y), Tryptophan (W); 7. Serine (S), Threonine (T); and 8. Cysteine (C), Methionine (M).

The terms “derived from” or “of” when used in reference to a nucleic acid or protein indicates that its sequence is identical or substantially identical to that of an organism of interest.

The term “corresponding” when used in reference to a bacterium, refers to a bacterium of the same genus and species as the bacterium of interest. For instance in regard to an S. elongates cell comprising a recombinant polynucleotide encoding a galactose transporter protein, a “corresponding bacterium” is an S. elongates cell (wild type, parental, or otherwise comparable) lacking the recombinant polynucleotide (e.g., or otherwise not expressing the galactose transporter protein.”

The terms “decrease,” “reduce” and “reduction” as used in reference to biological function (e.g., enzymatic activity, production of compound, expression of a protein, etc.) refer to a measurable lessening in the function by preferably at least 10%, more preferably at least 50%, still more preferably at least 75%, and most preferably at least 90%. Depending upon the function, the reduction may be from 10% to 100%. The term “substantial reduction” and the like refers to a reduction of at least 50%, 75%, 90%, 95% or 100%.

The terms “increase,” “elevate” and “elevation” as used in reference to biological function (e.g., enzymatic activity, production of compound, expression of a protein, etc.) refer to a measurable augmentation in the function by preferably at least 10%, more preferably at least 50%, still more preferably at least 75%, and most preferably at least 90%. Depending upon the function, the elevation may be from 10% to 100%; or at least 10-fold, 100-fold, or 1000-fold up to 100-fold, 1000-fold or 10,000-fold or more. The term “substantial elevation” and the like refers to an elevation of at least 50%, 75%, 90%, 95% or 100%.

The terms “isolated” and “purified” as used herein refers to a material that is removed from at least one component with which it is naturally associated (e.g., removed from its original environment). The term “isolated,” when used in reference to a biosythetically-produced chemical, refers to a chemical that has been removed from the culture medium of the bacteria that produced the chemical. As such an isolated chemical is free of extraneous or unwanted compounds (e.g., substrate molecules, bacterial components, etc.).

As used herein, the singular form “a”, “an”, and “the” includes plural references unless indicated otherwise. For example, “a” galactose transport protein includes one or more galactose transport proteins.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments. It is understood that aspects and embodiments described herein as “comprising” include “consisting” and/or “consisting essentially of” aspects and embodiments.

It is to be understood that, while the compositions and methods disclosed herein have been described in conjunction with the preferred embodiments thereof, the foregoing description is intended to illustrate and not limit the scope thereof as defined in the appended claims. Other aspects, advantages, and modifications within the scope thereof as defined in the appended claims will be apparent to those skilled in the art to which the present disclosure pertains.

The following Examples are merely illustrative and are not meant to limit any aspects of the present disclosure in any way.

### EXAMPLES

### Example 1

### Expression of Glucose Transporter Proteins in Synechococcus elongatus PCC7942

Photoautotrophic bacterial cells, such as cyanobacteria, can be developed as a platform for the conversion of renewable solar energy to commodity chemicals, including biofuels. To achieve this conversion, a model cyanobacterium, Synechococcus elongatus PCC7942, was previously engineered to produce isobutyraldehyde and isobutanol (see, PCT publication WO 2010/071851). However, S. elongatus is an obligate photoautotroph, strictly dependent on the generation of photosynthetically derived energy for growth, and thus incapable of biomass or product formation in the absence of light energy. In order for any cyanobacterial fuel conversion to be economically competitive, the light energy must be supplied from the sun, and thus is only available between about 9 to 16 hours per day. To improve this scenario, three S. elongatus strains were developed to each grow on glucose, sucrose, and xylose, respectively, during night time (i.e., dark phase of a diurnal cycle). To utilize glucose, sucrose, and xylose during night time, glucose-, sucrose-, and xylose-specific sugar transporter proteins were each introduced into S. elongatus PCC7942.

### Materials and Methods

Reagents. The saccharides glucose, fructose, sucrose, and xylose were obtained from Sigma-Aldrich (St. Louis, Mo.). IPTG was obtained from Fisher Scientific (Hanover Park, Ill.). Phusion polymerase was obtained from NEB (Ipswich, Mass.). KOD polymerase was obtained from EMD4Biosciences (San Diego, Calif.). Spectinomycin was obtained from MP Biomedicals (Santa Ana, Calif.). Oligonucleotides were synthesized from Integrated DNA Technologies, Inc. (San Diego, Calif.).

Culture conditions. All cyanobacterial strains were grown in a BG-11 medium (Rippka R, et al., (1979) Journal of General Microbiology 111(1):1-61) at 30° C. Cultures were maintained in a custom cabinet with dimensions 56 cm by 36 cm by 76 cm. This cabinet was outfitted with 2 CFL natural spectrum bulbs from Verilux, rated at 26 watts. Light fluorescence rates were 25 μE s⁻¹m−². A shaker maintained shaking with settings at 100 rpm. Pre-cultures for diurnal experiments were maintain for at least 72 hours in diurnal lighting conditions to ensure proper circadian rhythm. Growth assays used a total volume of 10 mL of culture in 30 mL test tubes. Cell growth was monitored by measuring OD₇₃₀.

For growth assays with the Synechococcus elongatus (S. elongates) strains, cells in exponential phase were diluted to an OD₇₃₀ of 0.2 in 10 mL BG-11 medium including 20 μg/mL spectinomycin and 0.1 mM IPTG. Wild-type assays omitted spectinomycin.

To test assays for contamination, bright field microscopy was utilized. Cells were counted within a Petroff-Hausser Counting Chamber slide to ensure constant volume throughout. Counting chambers were chosen randomly and green cells versus colorless cells were tallied. For all reported results, green cells were greater than 99% of the culture where n>500.

Plasmid construction. All S. elongatus strains and plasmids used in Example 1 are described in Table 1. All primers used are listed in Table 2.

TABLE 1
Strains, Plasmids and Genotypes

| Strain | Relevant Genotypes |
|---|---|
| AL257 | Synechococcus elongatus PCC7942 (wild-type) |
| AL358 | Ptrc: glcP integrated at NSI |
| AL360 | Ptrc: GLUT1 integrated at NSI |
| AL361 | Ptrc: galP integrated at NSI |
| AL434 | Ptrc: xylE-xylA-xylB integrated at NSI |
| AL504 | Ptrc: gfp integrated at NSI |
| AL505 | Ptrc: galP-gfp integrated at NSI |
| AL535 | Same as AL361, but ΔglgC |
| AL536 | Same as wild-type, but ΔglgC |
| AL1030 | Ptrc: cscK-cscB integrated at NSI |
| AL1067 | Ptrc: xylE integrated at NSI |
| Plasmid | Genotypes |
| pAM2991 | NSI targeting vector; Ptrc |
| pBBR1MCS-5 | gm^{r}; broad host range vector |
| pSA69 | P15A ori; amp^{r}; P_{LlacO1}::alsS-ilvC-ilvD |
| pAL18 | Same as pAM2991, but Ptrc: GLUT1; lacI^{q} |
| pAL40 | Same as pAM2991, but Ptrc: galP; lacI^{q} |
| pAL46 | Same as pAM2991, but Ptrc: glcP; lacI^{q} |
| pAL61 | Same as pAM2991, but Ptrc: gfp; lacI^{q} |
| pAL63 | Same as pAM2991, but Ptrc: gfp-galP; lacI^{q} |
| pAL65 | Same as pAM2991, but Ptrc: xylE; lacI^{q} |
| pAL70 | Same as pAM2991, but Ptrc: xylE-xylA-xylB; lacI^{q} |
| pAL82 | glgC knockout vector; gm^{r} |
| pAL288 | Same as pAM2991, but Ptrc: cscK-cscB; lacI^{q} |

TABLE 2
Primers

| Primer | Sequence (5′ -> 3′) |
|---|---|
| JM5 | CCGGAATTCAATACCCAGTATAATTCCAGTTATATATTTTCGA |
| JM6 | CGGGATCCATCCTAGGTTACAGCGTAGCAGTTTGTTGT |
| JM7 | CGCCTAGGAACTTTAAGAAGGAGATATACCATGCAAGCCTATT |
| | TTGACCAGCTCG |
| JM8 | CGGGATCCTTACGCCATTAATGGCAGAAGTTGC |
| JM55 | ATGGAATTCATGTCAGCCAAAGTATGGGT |
| JM56 | GGATCCATTGGGACGTCACCTCCTATATTGCTGAAGGTACAGG |
| JM57 | GAGGTGACGTCATGACGCAATCTCGATTGC |
| JM58 | TAGAGGATCCTTAACCCAGTTGCCAGAGTG |
| JM66 | ATGGAATTCATGGCACTGAATATTCCATT |
| MC127 | CTAACAATTGATGCCTGACGCTAAAAAACAGGGGCG |
| MC128 | CTATAGATCTTTAATCGTGAGCGCCTATTTCGCGCAGTT |
| MC186 | CTATCTCGAGTTAATCGTGAGCGCCTATTTCGCGCAGTT |
| MC187 | CATGCCTGACGCTAAAAAACAGGGGCGGTCA |
| MC188 | TTACGGCCGCTGCCACCGCCGCTACCGCCATCGTGAGCGCCTA |
| | TTTCGCGCAGTTTACG |
| MC189 | ACGATGGCGGTAGCGGCGGTGGCAGCGGCCGTAAAGGAGAAGA |
| | ACTTTTCACTGGAGTT |
| MC190 | CTTAGCATGCTTTGTATAGTTCATCCATGCCATG |
| MC191 | CTATGAATTCCGTAAAGGAGAAGAACTTTTCACTGGAGTT |
| MC192 | CTAAGGATCCTTAGCATGCTTTGTATAGTTCATCCATGCCATG |
| IM573 | GGTGCTAGCCACCGTGGAAACGGATGAAGG |
| IM574 | CATTTTTGTCGACGCCGGGAAGCCGATCTCG |
| IM581 | GAGTAGGTGGCTACGTCTCC |
| GR005 | CCGCTCGAGTACCAGCGATCCGTGTCCCTACTCG |
| GR006 | CGAGCACGCGTCAATTGCCCTAAGACAGTTGTCGTC |
| GR015 | CGCCGAACTGTTTGAACAGC |
| GR050 | TAGTAACCTCCAGCCTTTTTTGCC |

The galP, xylE, xylA, and xylB genes were isolated from E. coli genomic DNA (gDNA). The glcP gene was isolate from Synechocystis sp. PCC6803 gDNA (ATCC). The cscB and cscK genes were isolated from E. coli ATCC700927 (ATCC) gDNA. The glut1 gene was isolated from human erythrocytes. The GeneID accession number and nucleotide sequence of each gene are listed in Table 3.

TABLE 3
Genes and Origins

| Gene Name | Source | GeneID |
|---|---|---|
| galP | E. coli | 947434 |
| glcP | Synechocystis sp. PCC6803 | 952710 |
| glut1 | H. sapiens | 6513 |
| xylE | E. coli | 948529 |
| xylA | E. coli | 948141 |
| xylB | E. coli | 948133 |
| cscB | E. coli ATCC700927 | 958132 |
| cscK | E. coli ATCC700927 | 956713 |

The galP gene was amplified using primers MC 127 and MC 128, digested with MfeI and BgIII, and then ligated with pAM 2991 digested with EcoRI and BamHI to create pAL40. The xylE gene was amplified from JM05 and JM06, digested with EcoRI and BamHI and ligated with pAM2991 digested with the same enzymes, creating pAL65. The xylAB genes were amplified using JM07 and JM08 and digested with AvrII and BamHI then ligated to similarly digested pAL65 to create pAL70. The glcP gene was amplified using MC 170 and MC171, digested with BamHI and EcoRI, and ligated to similarly digested pAM 2991 to create pAL46. The cscB and cscK genes were amplified with JM55 and JM56, digested with EcoRI and BamHI, and ligated to similarly digested pAM2991 to create pAL289.

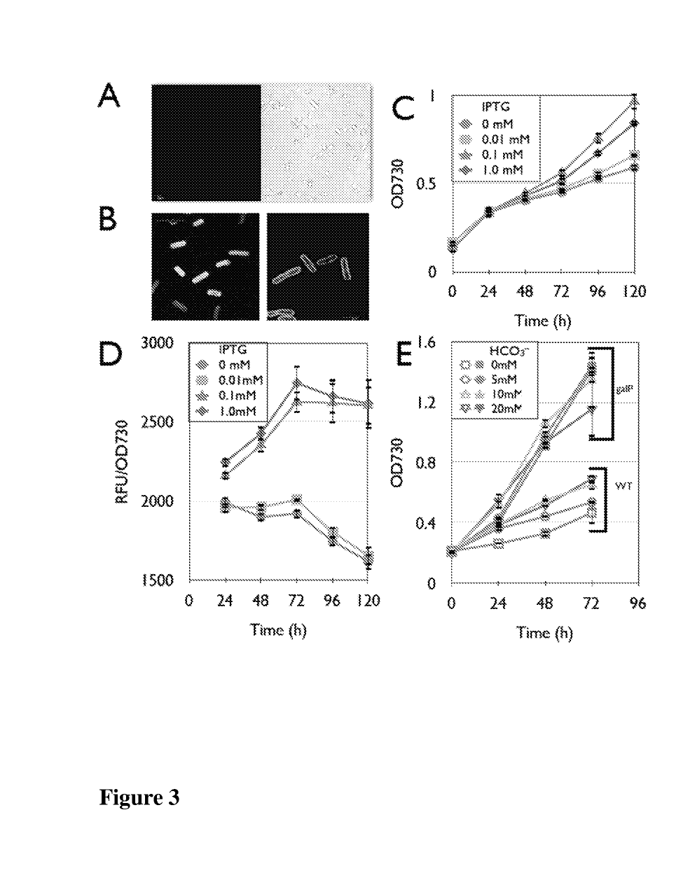

The pAL82 plasmid was constructed to delete the glgC gene from the S. elongatus chromosome (FIG. 3). The region for homologous recombination (590,459-593,751) was amplified from S. elongatus gDNA using primers GROOS and GRO06. The product was digested with XhoI and MluI and ligated with pSA69 (Atsumi S et al., (2008) Nature 451(7174):86-89) digested the same enzymes, creating pGR01. To clone the gentamicin resistance gene, pBBR1MCS-5 (Kovach Me et al., (1994) Biotechniques 16(5):800-802) was used as the PCR template with primers IM573 and IM574. The PCR product was digested with SalI and NheI and ligated with pGRO1 cut with the same enzyme, creating pAL82.

Transformation of S. elongates. Transformation of S. elongatus was performed as previously described (Golden S et al., (1987) Methods Enzymol 153:215-246). Strains were segregated several times by transferring colonies to fresh selective plates. Correct recombinants were confirmed by PCR to verify integration of targeting genes into the chromosome at NSI. The strains that were used and generated are listed in Table 1.

Confocal microscopy. All confocal microscopy images were taken using the Olympus America FV1000 system. A 488 nm laser was used for excitation of all mutants. Emission filter was set 500 nm-600 nm. Pinhole aperture was set to 100 μm. Laser % was set to 11.5%. Cells were placed in glass-bottom dishes for imaging.

Plate reader GFP assay. All GFP assays were conducted using a Microtek Synergy H1 plate reader (BioTek). BG-11 media only was used as a blank and excitation and emission wavelengths were set to 485 nm and 528 nm, respectively.

Glucose and xylose consumption assays. Glucose and xylose concentrations in the culture media were measured by a High Performance Liquid Chromatograph (Shimadzu) equipped Aminex HPX-87 column (Bio-Rad) and a refractive index detector. Samples were centrifuged and filtered using FiltrEX filter 96 plates (Corning).

### Results

Growth on glucose. We determined that Synechococcus elongatus (S. elongates) with the ability to grow under dark conditions by inducing the efficient uptake of sugars in S. elongates cells. Glucose is a common energy storage molecule in S. elongatus in the form of glycogen (Smith A J (1983) Ann Microbiol (Paris) 134B(1):93-113). Glycogen is built up within the cell throughout the light phase of metabolism then used as an energy source to maintain essential chemical process throughout the dark phase (Smith A J (1983) Ann Microbiol (Paris) 134B(1):93-113). Therefore, all the required genes for the breakdown of endogenous glucose should be present in S. elongatus.

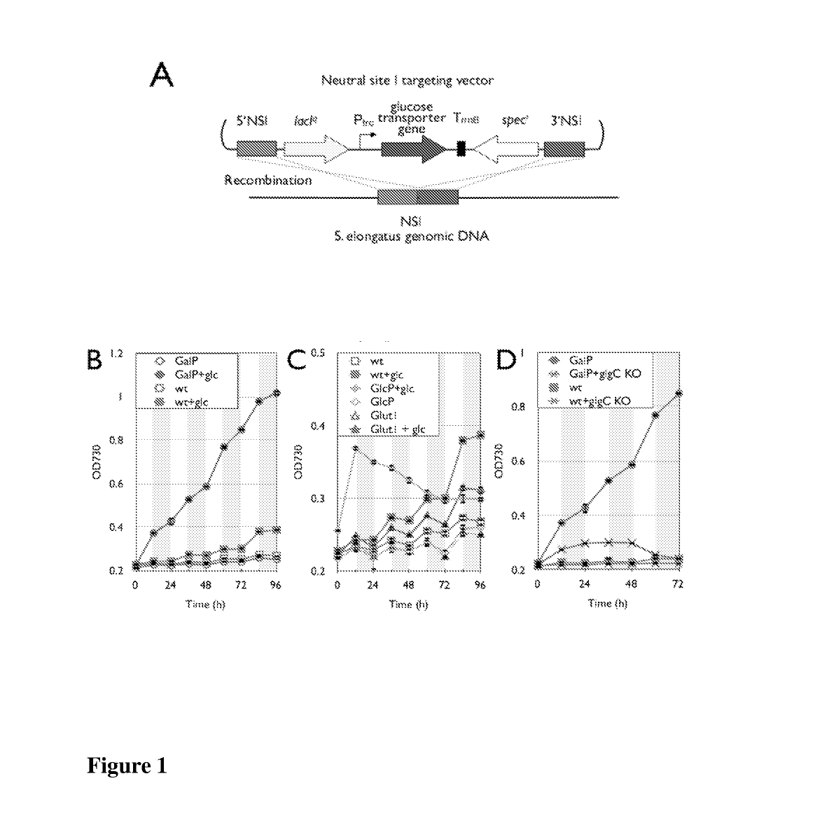

In order to engineer heterotrophic behavior in S. elongatus, we utilized heterologous genes encoding sugar transporter proteins in an attempt to confer heterotrophic behavior to S. elongatus. We integrated three glucose transporter proteins from a variety of organisms individually into the chromosome S. elongatus cells (FIG. 1). The three transporter proteins were the GlcP transporter protein from Synechocystis sp. PCC6803 (Zhang C C et al., (1989) Mol Microbiol 3(9):1221-1229), the GalP transporter protein from Escherichia coli (Hernandez-Montalvo V et al., (2003) Biotechnol Bioeng 83(6):687-694), and the Glut1 transporter protein from human erythrocytes (Mueckler M et al., (1985) Science 229(4717):941-945). Each gene was integrated into the S. elongatus chromosome at Neutral Site I (NSI) under the control of the isopropyl β-D-1-thiogalactopyranoside (IPTG) inducible promoter Ptrc (FIG. 1A). Growth of the resulting three strains and a wild-type control was measured under diurnal illumination conditions when cultured in the presence and absence of glucose (FIGS. 1B and 1C).

The galP strain. To amplify the growth difference between strains that could and could not grow on extracellular glucose, conditions were set so as to limit the amount of CO₂ and light intensity (25 μE/m²/s). The baseline growth rate of wild-type S. elongatus based on OD₇₃₀ was 0.161 day⁻¹ during the light cycle (48-60 h), with no growth during the dark cycle (FIG. 1B and Table 4). The wild-type control grew at a greater rate (0.204 day⁻¹) when cultured with glucose in the presence of light, yet exhibited no growth during the dark cycle (FIG. 1B). Growth rates for all periods are reported in Table 4. No changes in cellular morphology, such as size and shape of cells grown in the presence of absence of glucose were detected under microscopy analysis (1,000×).

In Table 4, the growth rates were calculated from OD₇₃₀ throughout diurnal conditions. All growth rates are reported in day⁻¹. In the table, “±” indicates the standard deviation. Any growth rate calculated to be less than 0.050 day⁻¹ was considered insignificant and shown in the table as not detectable (nd). In the table, “L” corresponds to light conditions; “D” corresponds to dark conditions; “OE” corresponds to overexpression; “KO” corresponds to deletion; “Xyl” corresponds to xylose; “Glc” corresponds to glucose; “Suc” corresponds to sucrose; and “n/a” corresponds to not analyzed.

TABLE 4
Growth Rates on Various Substrates

| | | | | 0-12 | 12-24 | 24-36 | 36-48 | 48-60 | 60-72 | 72-84 | 84-96 |
|---|---|---|---|---|---|---|---|---|---|---|---|
| Strain | OE | KO | Sugar | L | D | L | D | L | D | L | D |
| AL257 | | | | 0.082 ± | nd | 0.110 ± | nd | 0.161 ± | nd | 0.163 ± | nd |
| | | | | 0.064 | | 0.025 | | 0.030 | | 0.026 | |
| AL257 | | | Glc | 0.128 ± | nd | 0.238 ± | nd | 0.204 ± | nd | 0.469 ± | nd |
| | | | | 0.008 | | 0.017 | | 0.018 | | 0.012 | |
| AL536 | | glgC | Glc | nd | nd | nd | nd | nd | nd | n/a | n/a |
| AL257 | | | Suc | 0.298 ± | 0.081 ± | 0.319 ± | nd | 0.310 ± | 0.087 ± | 0.298 ± | 0.071 ± |
| | | | | 0.010 | 0.007 | 0.015 | | 0.017 | 0.027 | 0.025 | 0.019 |
| AL257 | | | Xyl | 0.149 ± | 0.104 ± | 0.285 ± | nd | 0.350 ± | 0.062 ± | 0.446 ± | 0.098 ± |
| | | | | 0.021 | 0.017 | 0.012 | | 0.017 | 0.012 | 0.016 | 0.016 |
| AL361 | galP | | | 0.128 ± | nd | 0.067 ± | nd | 0.126 ± | nd | 0.158 ± | nd |
| | | | | 0.017 | | 0.010 | | 0.018 | | 0.004 | |
| AL361 | galP | | Glc | 0.992 ± | 0.269 ± | 0.428 ± | 0.211 ± | 0.540 ± | 0.199 ± | 0.285 ± | 0.079 ± |
| | | | | 0.014 | 0.083 | 0.030 | 0.015 | 0.017 | 0.012 | 0.012 | 0.021 |
| AL535 | galP | glgC | Glc | 0.438 ± | 0.159 ± | nd | nd | Nd | nd | n/a | n/a |
| | | | | 0.016 | 0.013 | | | | | | |
| AL358 | glcP | | | 0.122 ± | nd | 0.118 ± | nd | 0.112 ± | nd | 0.279 ± | nd |
| | | | | 0.025 | | 0.036 | | 0.040 | | 0.049 | |
| AL358 | glcP | | Glc | 0.730 ± | nd | nd | nd | Nd | nd | nd | nd |
| | | | | 0.008 | | | | | | | |
| AL360 | GLUT1 | | | 0.140 ± | nd | 0.112 ± | nd | 0.115 ± | nd | 0.292 ± | nd |
| | | | | 0.018 | | 0.014 | | 0.029 | | 0.045 | |
| AL360 | GLUT1 | | Glc | 0.219 ± | nd | 0.212 ± | nd | 0.201 ± | nd | 0.352 ± | nd |
| | | | | 0.029 | | 0.027 | | 0.051 | | 0.055 | |
| AL1030 | cscKB | | | 0.134 ± | nd | 0.106 ± | nd | 0.141 ± | nd | 0.148 ± | nd |
| | | | | 0.023 | | 0.032 | | 0.038 | | 0.026 | |
| AL1030 | cscKB | | Suc | 0.327 ± | 0.157 ± | 0.401 ± | 0.122 ± | 0.376 ± | 0.128 ± | 0.412 ± | 0.126 ± |
| | | | | 0.029 | 0.021 | 0.026 | 0.034 | 0.031 | 0.011 | 0.008 | 0.009 |
| AL1067 | xylE | | | 0.102 ± | nd | 0.141 ± | nd | 0.161 ± | nd | 0.172 ± | nd |
| | | | | 0.004 | | 0.010 | | 0.020 | | 0.024 | |
| AL1067 | xylE | | Xyl | 0.140 ± | nd | 0.179 ± | nd | 0.154 ± | nd | 0.192 ± | nd |
| | | | | 0.021 | | 0.007 | | 0.015 | | 0.011 | |
| AL434 | xylEAB | | | nd | nd | 0.050 ± | nd | 0.121 ± | nd | 0.106 ± | nd |
| | | | | | | 0.011 | | 0.011 | | 0.014 | |
| AL434 | xylEAB | | Xyl | 0.395 ± | 0.351 ± | 0.572 ± | 0.336 ± | 0.471 ± | 0.291 ± | 0.313 ± | 0.082 ± |
| | | | | 0.026 | 0.014 | 0.025 | 0.012 | 0.012 | 0.020 | 0.015 | 0.020 |

The only recombinant S. elongatus strain to show a consistent increase of growth when cultured in the presence of glucose was the strain expressing the GalP transporter protein (FIG. 1B).

In addition to increased growth as compared to the wild-type control, the strain expressing the GalP transporter protein (the galP strain) showed growth even during the dark cycles (OD₇₃₀ of about 1), where the wild-type control showed no growth (OD₇₃₀ less than 0.4) (FIGS. 1B and 1C). The growth rate of the galP strain when cultured with glucose under light conditions was 0.540 day⁻¹, while the growth rate when cultured with glucose under dark conditions was 0.199 day⁻¹. The growth rate of the galP strain was 164% greater under light conditions in the presence of glucose than that of the wild-type control grown under the same conditions (0.204 day⁻¹). However, glucose consumption of the galP strain after 96 hours was not detectable with HPLC analysis. The dry weight of biomass increased only 0.1 g/L during the experiment.

The glcP and glut1 strains. The strain containing the GlcP transporter protein showed good growth during the first light cycle, but growth was arrested in the succeeding cycles (FIG. 1C). This result is consistent with previous results (Zhang C-C et al., (1998) FEMS Microbiol Lett 161(2):285-292; and Stebegg R et al., (2012) J Bacteriol 194(17):4601-4607). Additionally, the strain containing the Glut1 transporter protein exhibited impaired growth (FIG. 1C). All growth assays were confirmed to be free of contamination via microscope analysis. The above results demonstrate that S. elongatus can metabolize glucose for growth once glucose is transported into the cells.

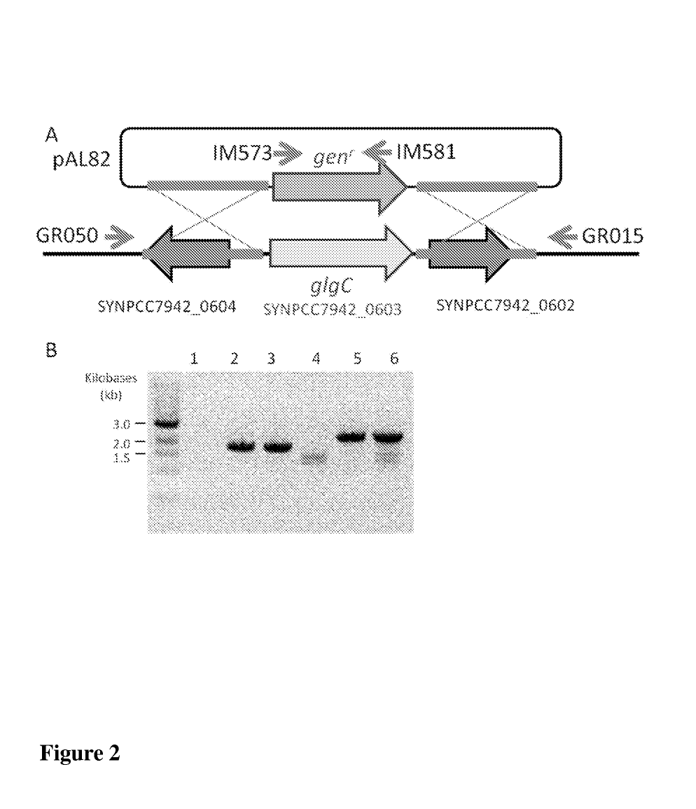

The galP-ΔglgC strain. Since S. elongatus naturally stores fixed carbon in the form of glycogen (Stanier R (1975) Biochem Soc Trans 3(3):352-359), we hypothesized that some portion of the transported glucose was captured and stored instead of contributing to cell growth. To test this possibility, we deleted a gene that is necessary for the formation of glycogen, glgC, from both the galP strain (the galP-ΔglgC strain) and the wild-type control (FIG. 2). We assayed these strains to test for any change in growth behavior. Both the galP-ΔglgC strain and the wild-type control containing the glgC deletion failed to grow significantly in the presence of glucose (FIG. 1D).

Characterization of galP expression. To characterize the expression of galP in the galP strain, gfp was fused to the 3′ end of galP (denoted as galP-gfp). The strains expressing galP-gfp or gfp alone were examined with fluorescent confocal microscopy (FIGS. 3A and 3B). The strain expressing galP-gfp showed a fluorescent signal only in the cellular membrane (FIG. 3B), while the gfp-expressing control strain showed a fluorescent signal throughout the cytoplasm (FIG. 3A) and the wild-type control strain showed no fluorescent signal (FIG. 3A). These results indicate that GalP transporter protein is successfully localized to the membrane of S. elongates, thus allowing efficient transport of glucose into the cell.

The galP and galP-gfp strains were also cultured with various concentrations of IPTG, and their growth was measured under continuous light conditions (FIG. 3C). The variation in IPTG concentration led to variation in growth of the cultures. This was also the case with the galP strain. However, the growth of the wild-type control was not altered by the addition of any amount of IPTG up to 1 mM. The optimum growth resulted from induction using 0.1 mM IPTG, while induction with 1 mM led to slightly lower cell growth (FIG. 3C).

Fluorescence intensity of the cultures of the galP-gfp strain was also measured throughout the growth assay (FIG. 3D). Time courses of GFP fluorescence intensity and OD₇₃₀ of the galP-gfp strain were measured with cells cultured with various concentrations of IPTG (FIG. 3D). Standardized fluorescent intensity (RFU/OD₇₃₀) indicated that culturing with 1 mM IPTG and 0.1 mM IPTG caused a similar level expression of the GalP-GFP fusion protein, and that expression from the P_{trc} promoter was saturated with 0.1 mM IPTG in this construct.

We also characterized the effects of bicarbonate on heterotrophic growth (FIG. 3E). This experiment was used to determine whether heterotrophic growth of the galP strain was measurable only due to the carbon limitations introduced by the assay conditions. The galP strain showed similar growth when cultured in the absence of bicarbonate, or in the presence of various concentrations of bicarbonate under continuous light conditions (FIG. 3E). However, higher concentrations of bicarbonate slightly enhanced growth of the wild-type control (FIG. 3E). The growth rate of the wild-type control increased to 0.398 day⁻¹ with 20 mM bicarbonate from 0.259 day⁻¹ when cultured without bicarbonate, while the growth rate of the galP strain did not change in the presence of bicarbonate (˜0.636 day⁻¹). These results suggest that carbon fixation is not the rate-limiting step for growth of the galP strain in the presence of 5 g/L glucose. However, carbon fixation appears to growth-limiting for the wild-type control in the presence of 5 g/L glucose.

Growth on sucrose. Sucrose is a natural metabolite in S. elongatus, and it has been shown to be synthesized in response to osmotic pressure (Ducat D C et al., (2012) Appl Environ Microbiol 78(8):2660-2668; and Suzuki E et al, (2010) Appl Environ Microbiol 76(10):3153-3159).

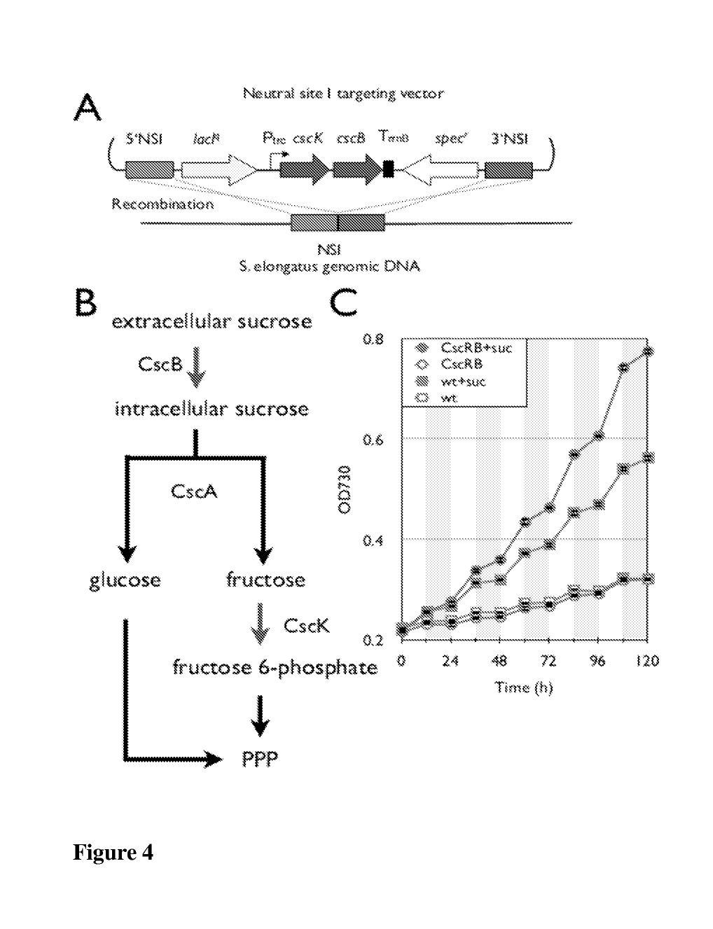

When cultured in the presence of sucrose, wild-type S. elongatus had an increased growth rate, 0.310 day⁻¹ under light conditions and 0.087 day⁻¹ under dark conditions, as compared the growth rate when cultured without sucrose under light conditions (0.161 day⁻¹) and no growth under dark conditions (FIG. 4C). This result demonstrates that S. elongatus is weakly permeable to sucrose and can utilize sucrose as a carbon source.

In order to improve S. elongatus growth rates with sucrose, the E. coli ATCC700927sucrose transporter gene cscB, and the E. coli ATCC700927 fructokinase gene cscK were integrated into the S. elongatus genome (FIG. 4A). This gene has been shown to be properly expressed in S. elongatus cells. This strain was constructed to more fully allow sucrose into the cell through the CscB transporter, and to be hydrolyzed to glucose and fructose by the endogenous sucrose invertase (encoded by SYNPCC7942_{—}0397). The fructokinase gene cscK was overexpressed to maximize the carbon flux to fructose-6-phosphate, a central metabolite in the oxidative pentose phosphate pathway (FIG. 4B).

The results showed that the cscK-cscB strain had an increased growth rate of 0.376 day⁻¹ under light conditions when cultured in the presences of 5 g/L sucrose (FIG. 4C). This represents an increase of approximately 21.3%, as compared to the growth rate of the wild-type control (0.310 day⁻¹). The results also showed that the cscK-cscB strain had an increased growth rate of 0.128 day⁻¹ under dark conditions when cultured in the presences of 5 g/L sucrose (FIG. 4C). This represents an increase of approximately 47.1%, as compared to the growth rate of the wild-type control (0.087 day⁻¹). Although sucrose enhanced the growth of the wild-type control, the effects of sucrose on growth were greater in the cscB-cscK strain than in the wild-type control.

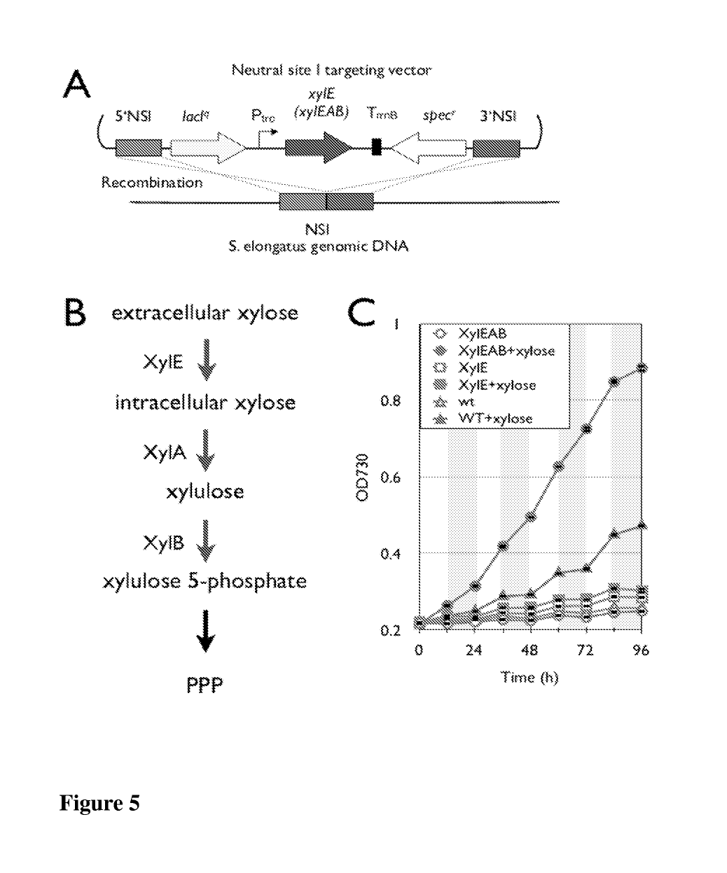

Growth on xylose. Xylose is the major part of abundantly available hemicellulosic biomass, and could be a promising inexpensive renewable feedstock for microbial production of biofuels and chemicals (Steen E J et al., (2010) Nature 463(7280):559-562). However, xylose is not a known metabolite of cyanobacteria, such as S. elongatus (FIG. 5).

To engineer S. elongatus to utilize xylose, the E. coli xylE gene, which encodes a xylose transporter, was integrated into S. elongatus (FIG. 5A). However, expression of the xylose transporter did not improve the growth of the strain when cultured in the presence of xylose under diurnal conditions (FIG. 5C).

Based on these results, we hypothesized that downstream xylose metabolism enzymes were missing from S. elongatus that allow the conversion of xylose to central metabolites. Based on S. elongatus genome sequence analysis, we determined that the S. elongatus genome does not contain the genes encoding for a xylose isomerase and a xylulokinase. Xylose isomerase and xylulokinase are responsible for the first two steps of xylose degradation (FIG. 5B).

Accordingly, we engineered a S. elongatus to express the E. coli genes encoding xylose isomerase and xylulokinase (xylA and xylB, respectively). To introduce xylose isomerase and xylulokinase into S. elongatus, an operon including the E. coli xylE, xylA, and xylB genes was integrated into the S. elongatus genome to generate the xylEAB strain (FIG. 5A).

This xylEAB strain was shown to grow heterotrophically under diurnal conditions (FIG. 5B). The xylE-xylA-xylB operon also allowed heterotrophic growth under dark conditions when the strain was cultured with xylose, while the wild-type control showed no growth when cultured with xylose under dark conditions (FIG. 5B). The xylEAB strain had a growth rate of 0.471 day⁻¹ under light conditions and 0.291 day⁻¹ under dark conditions when cultured in the presence of xylose. This was in contrast to the wild-type control, which had growth rates of 0.350 day⁻¹ and 0.062 day⁻¹ under the same respective conditions. Accordingly, the xylEAB strain had a growth rate under light conditions that was approximately 34.6% greater than the growth rate of the wild-type control grown under the same conditions. Moreover, the xylEAB strain had a growth rate under dark conditions that was approximately 369% greater than the growth rate of the wild-type control grown under the same conditions. The xylose consumption rate, which was measured by HPLC, averaged about 10mg h⁻¹ over the 96 h growth period.

Interestingly, the growth of the galP strain was faster than that of the xylEAB strain under light conditions when cultured in the presence of their respective sugars (FIGS. 1B and 5C). However, the growth of the xylEAB strain was faster than that of the galP strain under dark conditions when cultured in the presence of their respective sugars (FIGS. 1B and 5C).

### Example 2

### Expression of Sugar Transporter Proteins in Synechococcus elongatus PCC7942

Sugar transporters for alternative mono and disaccharide sugars are cloned into the Neutral Site I (NSI) of Synechococcus elongatus PCC7942 under the control of the P_{TRC} promoter, as described in Example 1. The cloned sugar transporters include, but are not limited to:
Glucose Transporters—ptsI/ptsH/ptsG/crr PTS system of Escherichia coli along with the GLUT3 MFS transporters of Homo sapiens.
Fructose Transporters—GLUT5 MFS transporter of Homo sapiens.
Mannose Transporters—manX/manY/manZ PTS system of Escherichia coli. Glucose specific transporters are also tested for their uptake of mannose.
Galactose Transporters—yjfF/ytfR/ytfT/ytfQ ABC system of Escherichia coli.
Xylose Transporters—the xylF/xylG/xylH ABC system of Escherichia coli.
Arabinose—araJ MFS transporter of Escherichia coli.
Sucrose—sacP PTS system of Bacillus subtilis.
Lactose—lacY MFS transporter of Escherichia coli.
Maltose—malE/malF/malG/malK ABC system of Escherichia coli.

### Example 3

### Integration of Metabolic Genes into Synechococcus elongatus PCC7942

Downstream metabolic genes are integrated into Synechococcus elongatus PCC7942 strains transformed with the sugar transporters of Example 2 to facilitate incorporation of sugars into their central metabolism. Integration is accomplished by cloning each of the sugar-specific metabolic genes, in various combinations containing a single gene or the complete list, upstream of their corresponding sugar transporter and integrating them into the NSI of Synechococcus elongatus PCC7942 under the control of the P_{TRC} promoter, as described in Example 1.

Below is a non-limiting list of sugars and their corresponding metabolic genes:

Glucose—Glucokinase (glk) of Escherichia coli for phosphorylation of glucose to glucose-6-phosphate.

Fructose—Manno(fructo)kinase (mak) of Escherichia coli and Fructokinase (cscK) of Escherichia coli EC3132 for phosphorylation of fructose to fructose-6-phosphate.

Mannose—Manno(fructo)kinase (mak) of Escherichia coli for the phosphorylation of mannose to mannose-6-phosphate, and for all systems Mannose-6-phosphate isomerase (manA) of Escherichia coli for the isomerization of mannose-6-phosphate to fructose-6-phosphate.

Galactose—Galactose-1-epimerase (galM) of Escherichia coli for the epimerization of β-D-galactose to α-D-galactose, Galactokinase (galK) of Escherichia coli for the phosphorylation of α-D-galactose to α-D-galactose-1-phosphate, Galactose-1-phosphate uridyltransferase (galT) of Escherichia coli for the conversion of α-D-galactose-1-phosphate to uridyldiphosphate (UDP)-D-galactose, and UDP-glucose-4-epimerase (galE) of Escherichia coli for the epimerization of UDP-D-galactose to UDP-D-glucose.

Arabinose—L-arabinose isomerase (araA) of Escherichia coli for the isomerization of L-arabinose to L-ribulose, L-ribulokinase (araB) of Escherichia coli for the phosphorylation of L-ribulose to L-ribulose-5-phosphate, and L-ribulose-5-phosphate-4-epimerase for the epimerization of L-ribulose-5-phosphate to xylulose-5-phoshate.

Sucrose—In conjunction with PTS transport systems, Sucrase-6-phosphate hydrolase (sacA) from Bacillus subtilis for the hydration of sucrose-6-phosphate to β-D-glucose-6-phosphate and β-D-fructose, along with the fructose degradation enzymes previously described. For use with MFS transport systems, Invertase (cscA) from Escherichia coli EC3132 for the hydration of sucrose to β-D-glucose and β-D-fructose, along with the fructose degradation enzymes previously described.

Maltose—Maltose phosphorylase (malP) from Lactococcus lactis for the phosphorylation of maltose to β-D-glucose and β-D-glucose-1-phosphate, and β-phosphoglucomutase (pgm) from Lactococcus lactis for the conversion of β-D-glucose-1-phosphate to β-D-glucose-6-phosphate.

Lactose—β-galactosidase (lacZ) from Escherichia coli for the hydration of lactose into β-D-galactose and β-D-glucose, along with the previously described galactose degradation enzymes.

### Example 4

### Combinations of Sugar Catabolic Pathways in Synechococcus elongatus PCC7942

After each of the sugar catabolic pathways described in Example 3 are cloned and characterized, different combinations of the catabolic pathways are constructed in Synechococcus elongatus PCC7942 to further expand the substrate utilization of individual strains.

Below is a non-limiting list of pathway combinations:
Glucose and Fructose Transport/Metabolism
Glucose and Xylose Transport/Metabolism
Glucose, Xylose, and Arabinose Transport/Metabolism
Glucose, Fructose, Xylose, and Arabinose Transport/Metabolism
Glucose, Fructose, Mannose, Galactose, Xylose, and Arabinose Transport/Metabolism
Sucrose and Lactose Transport/Metabolism

### Example 5

### Construction of Sugar Catabolic Pathways in Other Photoautotrophic or Photoheterotrophic Cyanobacteria

The sugar catabolic pathways described in Example 4 are constructed into other photoautotrophic or photoheterotrophic strains of cyanobacteria that include, without limitation, the thermostable cyanobacterium Thermosynechococcus elongatus BP-1, the marine cyanobacterium Synechococcus sp. WH8102, Synechococcus elongatus PCC7002, and Synechocystis sp PCC6803. As benchmarks, glucose pathways are constructed into the marine photoautotroph Synechococcus elongatus PCC7002 and the thermophilic freshwater photoautotroph Thermosynechococcus elongatus BP-1. The sugar utilization capacity of Synechocystis sp PCC6803 is expanded beyond glucose by incorporating the pathways for all monosaccharide and disaccharide pathways.

### Example 6

### Production of Commodity Chemicals in Synechococcus elongatus PCC7942

The production of commodity chemicals including, without limitation, biofuels, polymers, specialty chemicals, and pharmaceutical intermediates is increased in Synechococcus elongatus PCC7942 strains containing the various sugar transporters and sugar catabolic pathways described in Examples 1-4. Biofuels include, without limitation, alcohols such as ethanol, propanol, isopropanol, acetone, butanol, isobutanol, 2-methyl-1-butanol, 3-methyl-1-butanol, phenylethanol, fatty alcohols, and isopentenol; aldehydes, such as acetylaldehyde, propionaldehyde, butryaldehyde, isobutyraldehyde, 2-methyl-1-butanal, 3-methyl-1-butanal, phenylacetaldehyde, and fatty aldehydes; hydrocarbons, such as alkanes, alkenes, isoprenoids, fatty acids, wax esters, and ethyl esters; and inorganic fuels such as hydrogen. Polymers include, without limitation, 2,3-butanediol, 1,3-propandiol, 1,4-butanediol, polyhydroxyalkanoate, polyhydroxybutyrate, and isoprene. Specialty chemicals include, without limitation, carotenoids, such as lycopene, β-carotene, etc. Pharmaceutical intermediates include, without limitation, polyketides, statins, omega-3 fatty acids, isoprenoids, steroids, and erythromycin (antibiotic). Further examples of commodity chemicals include, without limitation, lactate, succinate, glutamate, citrate, malate, 3-hydroxypropionate, ascorbate, sorbitol, amino acids (leucine, valine, isoleucine, etc.), and hydroxybutyrate.

A strain of Synechococcus elongatus PCC7942 capable of metabolizing one or more mono- or disaccharide sugars is engineered to produce several biofuels.

The produced biofuels include, but are not limited to:

Ethanol: Integration of pyruvate decarboxylase (pdc) and alcohol dehydrogenase (adhB) from Zymomonas mobilis (or homologues thereof).

Isobutyraldehyde: Integration of 2-acetolactate synthase (alsS) from Bacillus subtilis, acetohydroxy acid isomeroreductase (ilvC) and dihydroxy acid dehydratase (ilvD) from Escherichia coli, and 2-ketoisovalerate decarboxylase (kivd) from Lactococcus lactis.

Isobutanol: Integration of all genes responsible for isobutyraldehyde production along with alcohol dehydrogenase (yqhD) from Escherichia coli.

Other Higher Chain Alcohols: 1-propanol, 1-butanol, 2-methyl-1-butanol, 3-methyl-1-butanol, and 2-phenylethanol minimally require 2-ketoisovalerate decarboxlase (kivd) from Lactococcus lactis and alcohol dehydrogenase (yqhD) from Escherichia coli.

2,3-Butanediol: Integration of 2-acetolactate synthase (alsS) from Bacillus subtilis, 2-acetolactate decarboxylase (alsD) from Aeromonas hydrophila, secondary alcohol dehydrogenase (adh) from Clostridium beijerinckii.

### Example 7

### Engineering a Synechococcus elongatus PCC7942 Strain for Unlimited Growth in Absence of Light

A Synechococcus elongatus PCC7942 strain is engineered to be capable of unlimited growth on a sugar substrate in the absence of light by random mutagenesis. Briefly, a chemical mutagen, such as N-methyl-N′-nitro-N-nitrosoguanidine (NTG) or ethylmethanesulfonate (EMS), is used to induce random point mutations across the genome of S. elongatus PCC7942 carrying a functional glucose transport pathway. After mutagenesis, cultures are enriched for a short time (1-2 weeks) under photoautotrophic conditions to ensure the preservation of photosynthetic capability. After this, cells are plated on BG-11 plates containing 10 g/L of glucose and incubated at 30° C. in the dark to select for strains able to grow on glucose in the absence of light. Strains capable of growth are verified by PCR to ensure they are derivatives of S. elongatus PCC7942, after which the entire genome is sequenced and further analyzed.

SEQUENCES
SEQ ID NO: 1: E. coli galP nucleotide sequence:
ATGCCTGACG CTAAAAAACA GGGGCGGTCA AACAAGGCAA TGACGTTTTT
CGTCTGCTTC CTTGCCGCTC TGGCGGGATT ACTCTTTGGC CTGGATATCG
GTGTAATTGC TGGCGCACTG CCGTTTATTG CAGATGAATT CCAGATTACT
TCGCACACGC AAGAATGGGT CGTAAGCTCC ATGATGTTCG GTGCGGCAGT
CGGTGCGGTG GGCAGCGGCT GGCTCTCCTT TAAACTCGGG CGCAAAAAGA
GCCTGATGAT CGGCGCAATT TTGTTTGTTG CCGGTTCGCT GTTCTCTGCG
GCTGCGCCAA ACGTTGAAGT ACTGATTCTT TCCCGCGTTC TACTGGGGCT
GGCGGTGGGT GTGGCCTCTT ATACCGCACC GCTGTACCTC TCTGAAATTG
CGCCGGAAAA AATTCGTGGC AGTATGATCT CGATGTATCA GTTGATGATC
ACTATCGGGA TCCTCGGTGC TTATCTTTCT GATACCGCCT TCAGCTACAC
CGGTGCATGG CGCTGGATGC TGGGTGTGAT TATCATCCCG GCAATTTTGC
TGCTGATTGG TGTCTTCTTC CTGCCAGACA GCCCACGTTG GTTTGCCGCC
AAACGCCGTT TTGTTGATGC CGAACGCGTG CTGCTACGCC TGCGTGACAC
CAGCGCGGAA GCGAAACGCG AACTGGATGA AATCCGTGAA AGTTTGCAGG
TTAAACAGAG TGGCTGGGCG CTGTTTAAAG AGAACAGCAA CTTCCGCCGC
GCGGTGTTCC TTGGCGTACT GTTGCAGGTA ATGCAGCAAT TCACCGGGAT
GAACGTCATC ATGTATTACG CGCCGAAAAT CTTCGAACTG GCGGGTTATA
CCAACACTAC CGAGCAAATG TGGGGGACCG TGATTGTCGG CCTGACCAAC
GTACTTGCCA CCTTTATCGC AATCGGCCTT GTTGACCGCT GGGGACGTAA
ACCAACGCTA ACGCTGGGCT TCCTGGTGAT GGCTGCTGGC ATGGGCGTAC
TCGGTACAAT GATGCATATC GGTATTCACT CTCCGTCGGC GCAGTATTTC
GCCATCGCCA TGCTGCTGAT GTTTATTGTC GGTTTTGCCA TGAGTGCCGG
TCCGCTGATT TGGGTACTGT GCTCCGAAAT TCAGCCGCTG AAAGGCCGCG
ATTTTGGCAT CACCTGCTCC ACTGCCACCA ACTGGATTGC CAACATGATC
GTTGGCGCAA CGTTCCTGAC CATGCTCAAC ACGCTGGGTA ACGCCAACAC
CTTCTGGGTG TATGCGGCTC TGAACGTACT GTTTATCCTG CTGACATTGT
GGCTGGTACC GGAAACCAAA CACGTTTCGC TGGAACATAT TGAACGTAAT
CTGATGAAAG GTCGTAAACT GCGCGAAATA GGCGCTCACG ATTAA
SEQ ID NO: 2: E. coli galP amino acid sequence:
MPDAKKQGRS NKAMTFFVCF LAALAGLLFG LDIGVIAGAL PFIADEFQIT
SHTQEWVVSS MMFGAAVGAV GSGWLSFKLG RKKSLMIGAI LFVAGSLFSA
AAPNVEVLIL SRVLLGLAVG VASYTAPLYL SEIAPEKIRG SMISMYQLMI
TIGILGAYLS DTAFSYTGAW RWMLGVIIIP AILLLIGVFF LPDSPRWFAA
KRRFVDAERV LLRLRDTSAE AKRELDEIRE SLQVKQSGWA LFKENSNFRR
AVFLGVLLQV MQQFTGMNVI MYYAPKIFEL AGYTNTTEQM WGTVIVGLTN
VLATFIAIGL VDRWGRKPTL TLGFLVMAAG MGVLGTMMHI GIHSPSAQYF
AIAMLLMFIV GFAMSAGPLI WVLCSEIQPL KGRDFGITCS TATNWIANMI
VGATFLTMLN TLGNANTFWV YAALNVLFIL LTLWLVPETK HVSLEHIERN
LMKGRKLREI GAHD
SEQ ID NO: 3: Synechocystis sp. PCC6803 glcP nucleotide
sequence:
ATGAATCCCT CCTCTTCTCC TTCCCAATCT ACGGCTAACG TTAAGTTTGT
CCTGCTGATT TCGGGGGTAG CAGCCCTGGG GGGGTTCCTG TTTGGCTTTG
ACACTGCGGT GATCAATGGG GCGGTGGCGG CCCTACAAAA ACATTTTCAG
ACGGACAGTC TTTTAACAGG TTTATCTGTA TCCTTAGCTC TGTTGGGATC
AGCACTGGGA GCCTTTGGGG CGGGACCGAT CGCCGATCGC CATGGGCGGA
TTAAAACGAT GATTTTAGCG GCGGTGCTGT TCACCCTCAG TTCCATTGGG
TCGGGTTTAC CTTTCACCAT TTGGGATTTT ATTTTTTGGC GGGTGTTGGG
GGGCATTGGG GTGGGGGCCG CTAGCGTTAT TGCCCCGGCC TACATTGCGG
AAGTGTCGCC GGCCCATCTG CGGGGGCGTT TAGGATCTTT GCAACAGTTG
GCCATTGTTT CTGGCATTTT CATTGCCCTG CTCAGTAATT GGTTTATTGC
TTTGATGGCG GGGGGATCGG CCCAAAATCC CTGGTTGTTC GGTGCGGCGG
CCTGGCGTTG GATGTTCTGG ACAGAGCTAA TTCCCGCCCT GCTCTATGGA
GTTTGCGCTT TCCTGATCCC CGAATCTCCC CGGTATTTAG TCGCCCAAGG
GCAAGGGGAA AAAGCGGCGG CTATTTTGTG GAAAGTGGAA GGGGGAGACG
TGCCCAGTCG CATTGAGGAA ATCCAGGCAA CGGTTAGTCT CGACCATAAA
CCCCGGTTTA GCGATCTGCT CAGTCGTCGG GGAGGATTAT TGCCCATTGT
CTGGATTGGT ATGGGGTTGT CGGCACTACA ACAGTTTGTT GGCATTAACG
TAATTTTTTA TTACAGTAGC GTGCTCTGGC GATCGGTGGG TTTTACCGAA
GAAAAGTCTC TGTTAATCAC GGTAATCACT GGTTTTATCA ATATCCTCAC
CACCCTAGTG GCGATCGCCT TTGTGGATAA ATTTGGCCGT AAGCCTTTGT
TGCTCATGGG CTCCATTGGT ATGACCATTA CCTTGGGCAT CCTTTCCGTG
GTGTTTGGGG GAGCAACGGT GGTTAATGGC CAACCCACCC TGACGGGGGC
CGCTGGGATA ATTGCTTTGG TGACAGCCAA TCTTTATGTA TTTAGTTTTG
GTTTTTCTTG GGGGCCCATT GTTTGGGTCT TGCTGGGGGA AATGTTTAAT
AACAAAATTC GAGCGGCGGC CCTATCGGTG GCGGCGGGGG TACAGTGGAT
TGCTAACTTT ATTATTTCCA CTACTTTTCC GCCCCTATTG GATACGGTGG
GTTTGGGCCC CGCCTATGGT TTATATGCCA CTTCAGCGGC CATTTCAATT
TTCTTTATCT GGTTTTTTGT GAAGGAAACT AAGGGTAAAA CTCTGGAGCA
AATGTGA
SEQ ID NO: 4: Synechocystis sp. PCC6803 glcP amino acid
sequence:
MNPSSSPSQS TANVKFVLLI SGVAALGGFL FGFDTAVING AVAALQKHFQ
TDSLLTGLSV SLALLGSALG AFGAGPIADR HGRIKTMILA AVLFTLSSIG
SGLPFTIWDF IFWRVLGGIG VGAASVIAPA YIAEVSPAHL RGRLGSLQQL
AIVSGIFIAL LSNWFIALMA GGSAQNPWLF GAAAWRWMFW TELIPALLYG
VCAFLIPESP RYLVAQGQGE KAAAILWKVE GGDVPSRIEE IQATVSLDHK
PRFSDLLSRR GGLLPIVWIG MGLSALQQFV GINVIFYYSS VLWRSVGFTE
EKSLLITVIT GFINILTTLV AIAFVDKFGR KPLLLMGSIG MTITLGILSV
VFGGATVVNG QPTLTGAAGI IALVTANLYV FSFGFSWGPI VWVLLGEMFN
NKIRAAALSV AAGVQWIANF IISTTFPPLL DTVGLGPAYG LYATSAAISI
FFIWFFVKET KGKTLEQM
SEQ ID NO: 5: H. sapiens glut1 nucleotide sequence:
ATGGAACCCAGCTCCAAGAAATTGACCGGACGCCTGATGCTGGCCGTTGGAGGGGCCGTGCT
GGGCTCGCTGCAGTTTGGCTACAACACCGGCGTGATCAATGCGCCGCAGAAAGTGATTGAAG
AATTTTACAACCAAACCTGGGTCCATCGCTACGGCGAGAGCATCCTGCCCACAACCCTCACC
ACCCTGTGGAGCCTGAGCGTCGCCATTTTTAGTGTGGGAGGTATGATCGGCAGCTTCTCCGT
GGGCTTGTTCGTCAATCGCTTTGGTCGGCGCAACTCCATGCTCATGATGAACCTGTTGGCCT
TTGTGTCTGCGGTCCTCATGGGCTTCAGCAAACTGGGAAAGTCGTTTGAAATGCTGATCCTC
GGTCGCTTCATTATCGGCGTGTACTGTGGCTTGACTACCGGCTTTGTTCCTATGTACGTGGG
GGAGGTGTCGCCCACCGCACTCCGCGGTGCGCTGGGTACGTTGCATCAATTGGGCATCGTGG
TGGGCATTCTCATCGCCCAGGTGTTTGGCCTGGATAGCATCATGGGTAACAAAGATTTGTGG
CCGCTGCTCCTGAGTATCATTTTCATCCCCGCACTGTTGCAATGTATCGTGCTCCCTTTTTG
CCCGGAATCCCCCCGTTTTCTCCTCATCAACCGCAACGAAGAAAACCGCGCCAAAAGCGTGC
TCAAAAAGTTGCGAGGGACCGCCGATGTGACTCACGACTTGCAGGAGATGAAAGAGGAGAGC
CGTCAGATGATGCGCGAGAAGAAGGTGACAATTCTGGAACTGTTCCGCAGCCCTGCGTACCG
CCAACCAATCTTGATTGCAGTCGTGCTGCAACTCAGCCAGCAGTTGAGCGGCATTAACGCTG
TCTTTTACTATTCCACCTCGATCTTTGAAAAAGCAGGCGTGCAGCAACCCGTCTACGCCACC
ATTGGATCGGGGATCGTGAATACCGCTTTTACCGTTGTGTCGCTGTTTGTCGTTGAACGAGC
TGGACGGCGAACTCTCCACCTGATTGGTCTGGCCGGGATGGCTGGATGCGCCATCCTGATGA
CCATTGCACTGGCCCTCCTGGAACAACTGCCCTGGATGAGCTACCTCTCTATTGTTGCCATC
TTTGGCTTCGTGGCGTTCTTTGAGGTTGGTCCGGGTCCAATCCCATGGTTTATCGTGGCTGA
ACTCTTTAGCCAGGGTCCACGTCCGGCTGCGATTGCTGTGGCAGGTTTTTCGAATTGGACGA
GTAACTTCATCGTCGGCATGTGTTTTCAATACGTCGAACAGCTCTGTGGCCCATACGTGTTT
ATCATCTTCACCGTCTTGCTCGTCCTGTTCTTTATCTTTACATACTTCAAAGTGCCCGAAAC
CAAAGGGCGGACCTTTGATGAAATCGCCAGCGGCTTTCGGCAGGGAGGAGCCAGCCAGAGCG
ATAAGACCCCAGAGGAGTTGTTTCACCCATTGGGGGCTGATAGCCAAGTGTGA
SEQ ID NO: 6: H. sapiens glut1 amino acid sequence:
MEPSSKKLTG RLMLAVGGAV LGSLQFGYNT GVINAPQKVI EEFYNQTWVH
RYGESILPTT LTTLWSLSVA IFSVGGMIGS FSVGLFVNRF GRRNSMLMMN
LLAFVSAVLM GFSKLGKSFE MLILGRFIIG VYCGLTTGFV PMYVGEVSPT
AFRGALGTLH QLGIVVGILI AQVFGLDSIM GNKDLWPLLL SIIFIPALLQ
CIVLPFCPES PRFLLINRNE ENRAKSVLKK LRGTADVTHD LQEMKEESRQ
MMREKKVTIL ELFRSPAYRQ PILIAVVLQL SQQLSGINAV FYYSTSIFEK
AGVQQPVYAT IGSGIVNTAF TVVSLFVVER AGRRTLHLIG LAGMAGCAIL
MTIALALLEQ LPWMSYLSIV AIFGFVAFFE VGPGPIPWFI VAELFSQGPR
PAAIAVAGFS NWTSNFIVGM CFQYVEQLCG PYVFIIFTVL LVLFFIFTYF
KVPETKGRTF DEIASGFRQG GASQSDKTPE ELFHPLGADS QV
SEQ ID NO: 7: E. coli XylE nucleotide sequence:
ATGAATACCC AGTATAATTC CAGTTATATA TTTTCGATTA CCTTAGTCGC
TACATTAGGT GGTTTATTAT TTGGCTACGA CACCGCCGTT ATTTCCGGTA
CTGTTGAGTC ACTCAATACC GTCTTTGTTG CTCCACAAAA CTTAAGTGAA
TCCGCTGCCA ACTCCCTGTT AGGGTTTTGC GTGGCCAGCG CTCTGATTGG
TTGCATCATC GGCGGTGCCC TCGGTGGTTA TTGCAGTAAC CGCTTCGGTC
GTCGTGATTC ACTTAAGATT GCTGCTGTCC TGTTTTTTAT TTCTGGTGTA
GGTTCTGCCT GGCCAGAACT TGGTTTTACC TCTATAAACC CGGACAACAC
TGTGCCTGTT TATCTGGCAG GTTATGTCCC GGAATTTGTT ATTTATCGCA
TTATTGGCGG TATTGGCGTT GGTTTAGCCT CAATGCTCTC GCCAATGTAT
ATTGCGGAAC TGGCTCCAGC TCATATTCGC GGGAAACTGG TCTCTTTTAA
CCAGTTTGCG ATTATTTTCG GGCAACTTTT AGTTTACTGC GTAAACTATT
TTATTGCCCG TTCCGGTGAT GCCAGCTGGC TGAATACTGA CGGCTGGCGT
TATATGTTTG CCTCGGAATG TATCCCTGCA CTGCTGTTCT TAATGCTGCT
GTATACCGTG CCAGAAAGTC CTCGCTGGCT GATGTCGCGC GGCAAGCAAG
AACAGGCGGA AGGTATCCTG CGCAAAATTA TGGGCAACAC GCTTGCAACT
CAGGCAGTAC AGGAAATTAA ACACTCCCTG GATCATGGCC GCAAAACCGG
TGGTCGTCTG CTGATGTTTG GCGTGGGCGT GATTGTAATC GGCGTAATGC
TCTCCATCTT CCAGCAATTT GTCGGCATCA ATGTGGTGCT GTACTACGCG
CCGGAAGTGT TCAAAACGCT GGGGGCCAGC ACGGATATCG CGCTGTTGCA
GACCATTATT GTCGGAGTTA TCAACCTCAC CTTCACCGTT CTGGCAATTA
TGACGGTGGA TAAATTTGGT CGTAAGCCAC TGCAAATTAT CGGCGCACTC
GGAATGGCAA TCGGTATGTT TAGCCTCGGT ACCGCGTTTT ACACTCAGGC
ACCGGGTATT GTGGCGCTAC TGTCGATGCT GTTCTATGTT GCCGCCTTTG
CCATGTCCTG GGGTCCGGTA TGCTGGGTAC TGCTGTCGGA AATCTTCCCG
AATGCTATTC GTGGTAAAGC GCTGGCAATC GCGGTGGCGG CCCAGTGGCT
GGCGAACTAC TTCGTCTCCT GGACCTTCCC GATGATGGAC AAAAACTCCT
GGCTGGTGGC CCATTTCCAC AACGGTTTCT CCTACTGGAT TTACGGTTGT
ATGGGCGTTC TGGCAGCACT GTTTATGTGG AAATTTGTCC CGGAAACCAA
AGGTAAAACC CTTGAGGAGC TGGAAGCGCT CTGGGAACCG GAAACGAAGA
AAACACAACA AACTGCTACG CTGTAA
SEQ ID NO: 8: E. coli xylE amino acid sequence:
MNTQYNSSYI FSITLVATLG GLLFGYDTAV ISGTVESLNT VFVAPQNLSE
SAANSLLGFC VASALIGCII GGALGGYCSN RFGRRDSLKI AAVLFFISGV
GSAWPELGFT SINPDNTVPV YLAGYVPEFV IYRIIGGIGV GLASMLSPMY
IAELAPAHIR GKLVSFNQFA IIFGQLLVYC VNYFIARSGD ASWLNTDGWR
YMFASECIPA LLFLMLLYTV PESPRWLMSR GKQEQAEGIL RKIMGNTLAT
QAVQEIKHSL DHGRKTGGRL LMFGVGVIVI GVMLSIFQQF VGINVVLYYA
PEVFKTLGAS TDIALLQTII VGVINLTFTV LAIMTVDKFG RKPLQIIGAL
GMAIGMFSLG TAFYTQAPGI VALLSMLFYV AAFAMSWGPV CWVLLSEIFP
NAIRGKALAI AVAAQWLANY FVSWTFPMMD KNSWLVAHFH NGFSYWIYGC
MGVLAALFMW KFVPETKGKT LEELEALWEP ETKKTQQTAT L
SEQ ID NO: 9: E. coli xylA nucleotide sequence:
ATGCAAGCCT ATTTTGACCA GCTCGATCGC GTTCGTTATG AAGGCTCAAA
ATCCTCAAAC CCGTTAGCAT TCCGTCACTA CAATCCCGAC GAACTGGTGT
TGGGTAAGCG TATGGAAGAG CACTTGCGTT TTGCCGCCTG CTACTGGCAC
ACCTTCTGCT GGAACGGGGC GGATATGTTT GGTGTGGGGG CGTTTAATCG
TCCGTGGCAG CAGCCTGGTG AGGCACTGGC GTTGGCGAAG CGTAAAGCAG
ATGTCGCATT TGAGTTTTTC CACAAGTTAC ATGTGCCATT TTATTGCTTC
CACGATGTGG ATGTTTCCCC TGAGGGCGCG TCGTTAAAAG AGTACATCAA
TAATTTTGCG CAAATGGTTG ATGTCCTGGC AGGCAAGCAA GAAGAGAGCG
GCGTGAAGCT GCTGTGGGGA ACGGCCAACT GCTTTACAAA CCCTCGCTAC
GGCGCGGGTG CGGCGACGAA CCCAGATCCT GAAGTCTTCA GCTGGGCGGC
AACGCAAGTT GTTACAGCGA TGGAAGCAAC CCATAAATTG GGCGGTGAAA
ACTATGTCCT GTGGGGCGGT CGTGAAGGTT ACGAAACGCT GTTAAATACC
GACTTGCGTC AGGAGCGTGA ACAACTGGGC CGCTTTATGC AGATGGTGGT
TGAGCATAAA CATAAAATCG GTTTCCAGGG CACGTTGCTT ATCGAACCGA
AACCGCAAGA ACCGACCAAA CATCAATATG ATTACGATGC CGCGACGGTC
TATGGCTTCC TGAAACAGTT TGGTCTGGAA AAAGAGATTA AACTGAACAT
TGAAGCTAAC CACGCGACGC TGGCAGGTCA CTCTTTCCAT CATGAAATAG
CCACCGCCAT TGCGCTTGGC CTGTTCGGTT CTGTCGACGC CAACCGTGGC
GATGCGCAAC TGGGCTGGGA CACCGACCAG TTCCCGAACA GTGTGGAAGA
GAATGCGCTG GTGATGTATG AAATTCTCAA AGCAGGCGGT TTCACCACCG
GTGGTCTGAA CTTCGATGCC AAAGTACGTC GTCAAAGTAC TGATAAATAT
GATCTGTTTT ACGGTCATAT CGGCGCGATG GATACGATGG CACTGGCGCT
GAAAATTGCA GCGCGCATGA TTGAAGATGG CGAGCTGGAT AAACGCATCG
CGCAGCGTTA TTCCGGCTGG AATAGCGAAT TGGGCCAGCA AATCCTGAAA
GGCCAAATGT CACTGGCAGA TTTAGCCAAA TATGCTCAGG AACATCATTT
GTCTCCGGTG CATCAGAGTG GTCGCCAGGA ACAACTGGAA AATCTGGTAA
ACCATTATCT GTTCGACAAA TAA
SEQ ID NO: 10: E. coli xylA amino acid sequence:
MQAYFDQLDR VRYEGSKSSN PLAFRHYNPD ELVLGKRMEE HLRFAACYWH
TFCWNGADMF GVGAFNRPWQ QPGEALALAK RKADVAFEFF HKLHVPFYCF
HDVDVSPEGA SLKEYINNFA QMVDVLAGKQ EESGVKLLWG TANCFTNPRY
GAGAATNPDP EVFSWAATQV VTAMEATHKL GGENYVLWGG REGYETLLNT
DLRQEREQLG RFMQMVVEHK HKIGFQGTLL IEPKPQEPTK HQYDYDAATV
YGFLKQFGLE KEIKLNIEAN HATLAGHSFH HEIATAIALG LFGSVDANRG
DAQLGWDTDQ FPNSVEENAL VMYEILKAGG FTTGGLNFDA KVRRQSTDKY
DLFYGHIGAM DTMALALKIA ARMIEDGELD KRIAQRYSGW NSELGQQILK
GQMSLADLAK YAQEHHLSPV HQSGRQEQLE NLVNHYLFDK
SEQ ID NO: 11: E. coli xylB nucleotide sequence:
ATGTATATCG GGATAGATCT TGGCACCTCG GGCGTAAAAG TTATTTTGCT
CAACGAGCAG GGTGAGGTGG TTGCTGCGCA AACGGAAAAG CTGACCGTTT
CGCGCCCGCA TCCACTCTGG TCGGAACAAG ACCCGGAACA GTGGTGGCAG
GCAACTGATC GCGCAATGAA AGCTCTGGGC GATCAGCATT CTCTGCAGGA
CGTTAAAGCA TTGGGTATTG CCGGCCAGAT GCACGGAGCA ACCTTGCTGG
ATGCTCAGCA ACGGGTGTTA CGCCCTGCCA TTTTGTGGAA CGACGGGCGC
TGTGCGCAAG AGTGCACTTT GCTGGAAGCG CGAGTTCCGC AATCGCGGGT
GATTACCGGC AACCTGATGA TGCCCGGATT TACTGCGCCT AAATTGCTAT
GGGTTCAGCG GCATGAGCCG GAGATATTCC GTCAAATCGA CAAAGTATTA
TTACCGAAAG ATTACTTGCG TCTGCGTATG ACGGGGGAGT TTGCCAGCGA
TATGTCTGAC GCAGCTGGCA CCATGTGGCT GGATGTCGCA AAGCGTGACT
GGAGTGACGT CATGCTGCAG GCTTGCGACT TATCTCGTGA CCAGATGCCC
GCATTATACG AAGGCAGCGA AATTACTGGT GCTTTGTTAC CTGAAGTTGC
GAAAGCGTGG GGTATGGCGA CGGTGCCAGT TGTCGCAGGC GGTGGCGACA
ATGCAGCTGG TGCAGTTGGT GTGGGAATGG TTGATGCTAA TCAGGCAATG
TTATCGCTGG GGACGTCGGG GGTCTATTTT GCTGTCAGCG AAGGGTTCTT
AAGCAAGCCA GAAAGCGCCG TACATAGCTT TTGCCATGCG CTACCGCAAC
GTTGGCATTT AATGTCTGTG ATGCTGAGTG CAGCGTCGTG TCTGGATTGG
GCCGCGAAAT TAACCGGCCT GAGCAATGTC CCAGCTTTAA TCGCTGCAGC
TCAACAGGCT GATGAAAGTG CCGAGCCAGT TTGGTTTCTG CCTTATCTTT
CCGGCGAGCG TACGCCACAC AATAATCCCC AGGCGAAGGG GGTTTTCTTT
GGTTTGACTC ATCAACATGG CCCCAATGAA CTGGCGCGAG CAGTGCTGGA
AGGCGTGGGT TATGCGCTGG CAGATGGCAT GGATGTCGTG CATGCCTGCG
GTATTAAACC GCAAAGTGTT ACGTTGATTG GGGGCGGGGC GCGTAGTGAG
TACTGGCGTC AGATGCTGGC GGATATCAGC GGTCAGCAGC TCGATTACCG
TACGGGGGGG GATGTGGGGC CAGCACTGGG CGCAGCAAGG CTGGCGCAGA
TCGCGGCGAA TCCAGAGAAA TCGCTCATTG AATTGTTGCC GCAACTACCG
TTAGAACAGT CGCATCTACC AGATGCGCAG CGTTATGCCG CTTATCAGCC
ACGACGAGAA ACGTTCCGTC GCCTCTATCA GCAACTTCTG CCATTAATGG
CGTAA
SEQ ID NO: 12: E. coli xylB amino acid sequence:
MYIGIDLGTS GVKVILLNEQ GEVVAAQTEK LTVSRPHPLW SEQDPEQWWQ
ATDRAMKALG DQHSLQDVKA LGIAGQMHGA TLLDAQQRVL RPAILWNDGR
CAQECTLLEA RVPQSRVITG NLMMPGFTAP KLLWVQRHEP EIFRQIDKVL
LPKDYLRLRM TGEFASDMSD AAGTMWLDVA KRDWSDVMLQ ACDLSRDQMP
ALYEGSEITG ALLPEVAKAW GMATVPVVAG GGDNAAGAVG VGMVDANQAM
LSLGTSGVYF AVSEGFLSKP ESAVHSFCHA LPQRWHLMSV MLSAASCLDW
AAKLTGLSNV PALIAAAQQA DESAEPVWFL PYLSGERTPH NNPQAKGVFF
GLTHQHGPNE LARAVLEGVG YALADGMDVV HACGIKPQSV TLIGGGARSE
YWRQMLADIS GQQLDYRTGG DVGPALGAAR LAQIAANPEK SLIELLPQLP
LEQSHLPDAQ RYAAYQPRRE TFRRLYQQLL PLMA
SEQ ID NO: 13: E. coli ATCC700927 cscB nucleotide sequence:
ATGGCACTGA ATATTCCATT CAGAAATGCG TACTATCGTT TTGCATCCAG
TTACTCATTT CTCTTTTTTA TTTCCTGGTC GCTGTGGTGG TCGTTATACG
CTATTTGGCT GAAAGGACAT CTAGGGTTGA CAGGGACGGA ATTAGGTACA
CTTTATTCGG TCAACCAGTT TACCAGCATT CTATTTATGA TGTTCTACGG
CATCGTTCAG GATAAACTCG GTCTGAAGAA ACCGCTCATC TGGTGTATGA
GTTTCATCCT GGTCTTGACC GGACCGTTTA TGATTTACGT TTATGAACCG
TTACTGCAAA GCAATTTTTC TGTAGGTCTA ATTCTGGGGG CGCTCTTTTT
TGGCCTGGGG TATCTGGCGG GATGCGGTTT GCTTGACAGC TTCACCGAAA
AAATGGCGCG AAATTTTCAT TTCGAATATG GAACAGCGCG CGCCTGGGGA
TCTTTTGGCT ATGCTATTGG CGCGTTCTTT GCCGGCATAT TTTTTAGTAT
CAGTCCCCAT ATCAACTTCT GGTTGGTCTC GCTATTTGGC GCTGTATTTA
TGATGATCAA CATGTGTTTT AAAGATAAGG ATCACCAGTG CGTAGCGGCG
GATGCGGGAG GGGTAAAAAA AGAGGATTTT ATCGCAGTTT TCAAGGATCG
AAACTTCTGG GTTTTCGTCA TATTTATTGT TGGGACGTGG TCTTTCTATA
ACATTTTTGA TCAACAACTT TTTCCTGTCT TTTATGCAGG TTTATTCGAA
TCACACGATG TAGGAACGCG CCTGTATGGT TATCTCAACT CATTCCAGGT
GGTACTCGAA GCGCTGTGCA TGGCGATTAT TCCGTTCTTT GTGAATCGGG
TAGGGCCAAA AAATGCATTA CTTATCGGTG TTGTGATTAT GGCGTTGCGT
ATCCTTTCCT GCGCGCTGTT CGTTAACCCC TGGATTATTT CATTAGTGAA
GCTGTTACAT GCCATTGAGG TTCCACTTTG TGTCATATCC GTCTTCAAAT
ACAGCGTGGC AAACTTTGAT AAGCGCCTGT CGTCGACGAT CTTTCTGATT
GGTTTTCAAA TTGCCAGTTC GCTTGGGATT GTGCTGCTTT CAACGCCGAC
TGGGATACTC TTTGACCACG CAGGCTACCA GACAGTTTTC TTCGCAATTT
CGGGTATTGT CTGCCTGATG TTGCTATTTG GCATTTTCTT CCTGAGTAAA
AAACGCGAGC AAATAGTTAT GGAAACGCCT GTACCTTCAG CAATATAG
SEQ ID NO: 14: E. coli ATCC700927 cscB amino acid sequence:
MALNIPFRNA YYRFASSYSF LFFISWSLWW SLYAIWLKGH LGLTGTELGT
LYSVNQFTSI LFMMFYGIVQ DKLGLKKPLI WCMSFILVLT GPFMIYVYEP
LLQSNFSVGL ILGALFFGLG YLAGCGLLDS FTEKMARNFH FEYGTARAWG
SFGYAIGAFF AGIFFSISPH INFWLVSLFG AVFMMINMCF KDKDHQCVAA
DAGGVKKEDF IAVFKDRNFW VFVIFIVGTW SFYNIFDQQL FPVFYAGLFE
SHDVGTRLYG YLNSFQVVLE ALCMAIIPFF VNRVGPKNAL LIGVVIMALR
ILSCALFVNP WIISLVKLLH AIEVPLCVIS VFKYSVANFD KRLSSTIFLI
GFQIASSLGI VLLSTPTGIL FDHAGYQTVF FAISGIVCLM LLFGIFFLSK
KREQIVMETP VPSAI
SEQ ID NO: 15: E. coli ATCC700927 cscK nucleotide sequence:
ATGTCAGCCA AAGTATGGGT TTTAGGGGAT GCGGGTCGTA GATCTCTTGC
CAGAATCAGA CGGGCGGNWT ACTGCCTTGT CCTGGCGGCG CGCCAGCTAA
CGTTGCCGGT GGGAATCGCC AGATTAGGCG GAACAAGTGG GTTTATAGGT
CGGGTGGGGG ATGATCCTTT TGGTGCATTA ATGCAAAGAA CGCTGCTAAC
TGAGGGAGTC GATATCACGT ATCTGAAGCA AGATGAATGG CACCGGACAT
CCACGGTGCT TGTCGATCTG AACGATCAAG GGGAACGTTC ATTTACGTTT
ATGGTCCGCC CCAGTGCCGA TCTTTTTTTA GAGACGACAG ACTTGCCCTG
CTGGCGACAT GGCGAATGGT TACATCTCTG TTCAATTGCG TTGTCTGCCG
AGCCTTCGCG TACCAGCGCA TTTACTGCGA TGACGGAGAT CCGGCATGCC
GGAGGTTTTG TCAGCTTCGA TCCCAATATT CGTGAAGATC TATGGCAAGA
CGAGCATTTG CTCCGCTTGT GTTTGCGGCA GGCGCTACAA CTGGCGGATG
TCGTCAAGCT CTCGGAAGAA GAATGGCGAC TTATCAGTGG AAAAACACAG
AACGATCGGG ATATATGCGC CCTGGCAAAA GAGTATGAGA TCGCCATGCT
GTTGGTGACT AAAGGTGCAG AAGGGGTGGT GGTCTGTTAT CGAGGACAAG
TTCACCATTT TGCTGGAATG TCTGTGGATT GTGTCGATAG CACGGGGGCG
GGAGATGCGT TCGTTGCCGG GTTACTCACA GGTCTGTCCT CTACGGGATT
ATCTACAGAT GAGAGAGAAA TGCGACGAAT TATCGATCTC GCTCAACGTT
GCGGAGCGCT TGCAGTAACG GCGAAAGGGG CAATGACAGC GCTGCCATGT
CGACAAGAAC TGGAATAG
SEQ ID NO: 16: E. coli ATCC700927cscB amino acid sequence:
MSAKVWVLGD AGRRSLARIR RAXYCLVLAA RQLTLPVGIA RLGGTSGFIG
RVGDDPFGAL MQRTLLTEGV DITYLKQDEW HRTSTVLVDL NDQGERSFTF
MVRPSADLFL ETTDLPCWRH GEWLHLCSIA LSAEPSRTSA FTAMTEIRHA
GGFVSFDPNI REDLWQDEHL LRLCLRQALQ LADVVKLSEE EWRLISGKTQ
NDRDICALAK EYEIAMLLVT KGAEGVVVCY RGQVHHFAGM SVDCVDSTGA
GDAFVAGLLT GLSSTGLSTD EREMRRIIDL AQRCGALAVT AKGAMTALPC
RQELE

We claim:

1. An isolated bacterial cell of a photoautotrophic species, comprising a recombinant polynucleotide encoding a galactose transporter protein, wherein expression of the galactose transporter protein results in transport of glucose into the bacterial cell to increase growth of the bacterial cell on glucose under dark or diurnal conditions as compared to a corresponding photoautotrophic bacterial cell lacking the recombinant polynucleotide.

2. The isolated bacterial cell of claim 1, wherein the recombinant polynucleotide encodes a galactose transporter protein selected from the group consisting of a bacterial galP transporter protein, a eukaryotic galP transporter protein, a fungal galP transporter protein, a mammalian galP transporter protein, a bacterial Major Facilitator Superfamily (MFS) transporter protein, a eukaryotic MFS transporter protein, a fungal MFS transporter protein, a mammalian MFS transporter protein, a bacterial ATP-Binding Cassette Superfamily (ABC) transporter protein, a eukaryotic ABC transporter protein, a fungal ABC transporter protein, a mammalian ABC transporter protein, a bacterial Phosphotransferase System (PTS) transporter protein, a eukaryotic PTS transporter protein, a fungal PTS transporter protein, a mammalian PTS transporter protein, and a homolog thereof.

3. The isolated bacterial cell of claim 1, wherein the recombinant polynucleotide encodes an E. coli galP transporter protein.

4. An isolated bacterial cell of a photoautotrophic species, comprising a recombinant polynucleotide encoding a disaccharide sugar transporter protein, wherein expression of the disaccharide sugar transporter protein results in transport of a disaccharide sugar into the bacterial cell to increase growth of the bacterial cell on the disaccharide sugar under dark or diurnal conditions as compared to a corresponding photoautotrophic bacterial cell lacking the recombinant polynucleotide.

5. The bacterial cell of claim 4, wherein the recombinant polynucleotide encodes a disaccharide sugar transporter protein selected from the group consisting of a sucrose transporter protein, a lactose transporter protein, a lactulose transporter protein, a maltose transporter protein, a trehalose transporter protein, a cellobiose transporter protein, and a homolog thereof.

6. The bacterial cell of claim 4, wherein the recombinant polynucleotide encodes a sucrose transporter protein.

7. The bacterial cell of claim 6, wherein the sucrose transporter protein is selected from the group consisting of an E. coli CscB sucrose transporter protein, a B. subtilis SacP transporter protein, a Brassica napus Sut1 transporter protein, a Juglans regia Sut1 transporter protein, an Arabidopsis thaliana Suc6 transporter protein, an Arabidopsis thaliana SUT4 transporter protein, a Drosophila melanogaster Slc45-1 transporter protein, and a Dickeya dadantii ScrA transporter protein.

8. The bacterial cell of claim 6, wherein the sucrose transporter protein is an E. coli CscB sucrose transporter protein.

9. The bacterial cell of claim 4, wherein the bacterial cell further comprises at least one additional recombinant polynucleotide encoding a fructokinase protein.

10. The bacterial cell of claim 9, wherein the fructokinase protein is selected from the group consisting of an E. coli CscK fructokinase protein, a Lycopersicon esculentum Frk1 fructokinase protein, a Lycopersicon esculentum Frk2 fructokinase protein, a H. sapiens KHK fructokinase protein, an A. thaliana FLN-1 fructokinase protein, an A. thaliana and FLN-2 fructokinase protein, a Yersinia pestis biovar Microtus str. 91001 NagC1 fructokinase protein, a Yersinia pseudotuberculosis YajF fructokinase protein, and a Natronomonas pharaonis Suk fructokinase protein.

11. The bacterial cell of claim 9, wherein the fructokinase protein is an E. coli CscK fructokinase protein.

12. An isolated bacterial cell of a photoautotrophic species, comprising a recombinant polynucleotide encoding a xylose transporter protein, wherein expression of the xylose transporter protein results in transport of xylose into the bacterial cell to increase growth of the bacterial cell on xylose under dark or diurnal conditions as compared to a corresponding photoautotrophic bacterial cell lacking the recombinant polynucleotide.

13. The isolated bacterial cell of claim 12, wherein the recombinant polynucleotide encodes a xylose transporter protein selected from the group consisting of an E. coli XylE xylose transporter protein, an E. coli xylF/xylG/xylH ABC xylose transporter protein, a Candida intermedia Gxf1 transporter protein, a Pichia stipitis Sut1 transporter protein, and an A. thaliana At5g59250 transporter protein.

14. The isolated bacterial cell of claim 12, wherein the recombinant polynucleotide encodes an E. coli XylE xylose transporter protein.

15. The isolated bacterial cell of claim 12, wherein the bacterial cell further comprises at least one additional recombinant polynucleotide encoding a xylose isomerase.

16. The isolated bacterial cell of claim 12, wherein the bacterial cell further comprises at least one additional recombinant polynucleotide encoding a xylulokinase.

17. The isolated bacterial cell of claim 12, wherein the bacterial cell further comprises a second recombinant polynucleotide encoding a xylose isomerase and a third recombinant polynucleotide encoding a xylulokinase.

18. The isolated bacterial cell of claim 12, wherein the recombinant polynucleotide further encodes a xylose isomerase and a xylulokinase.

19. The isolated bacterial cell of claim 15 wherein the xylose isomerase is selected from the group consisting of an E. coli XylA xylose isomerase, an A. thaliana AT5G57655 xylose isomerase, an Aspergillus niger XyrA xylose isomerase, and a Hypocrea jecorina Xyl1 xylose isomerase.

20. The isolated bacterial cell of claim 19, wherein the xylose isomerase is an E. coli XylA xylose isomerase.

21. The isolated bacterial cell of claim 16, wherein the xylulokinase is selected from the group consisting of an E. coli XylB xylulokinase, an Arabidopsis thaliana XK-1 xylulokinase, an Arabidopsis thaliana XK-2 xylulokinase, an E. coli LynK xylulokinase, a Streptomyces coelicolor SCO1170 xylulokinase, a Pseudomonas aeruginosa MtlY xylulokinase, a Yersinia pseudotuberculosis SgbK xylulokinase, and an E. coli AtlK xylulokinase.

22. The isolated bacterial cell of claim 21, wherein the xylulokinase is an E. coli XylB xylulokinase.

23. The isolated bacterial cell of any one of claims 1-22, wherein the recombinant polynucleotide and/or at least one additional recombinant polynucleotide is stably integrated into the genome of the bacterial cell.

24. The isolated bacterial cell of any one of claims 1-22, wherein the bacterial cell further comprises at least one additional recombinant polynucleotide encoding a sugar transport protein, wherein expression of the sugar transporter protein results in transport of sugar into the bacterial cell.

25. The isolated bacterial cell of claim 24, wherein the sugar is selected from the group consisting of a hexose, galactose, glucose, fructose, mannose, a disaccharide, sucrose, lactose, lactulose, maltose, trehalose, cellobiose, a pentose, xylose, arabinose, ribose, ribulose, and xylulose.

26. The isolated bacterial cell of any one of claims 1-22, wherein the bacterial cell further comprises the proteins necessary for the photoautotrophic bacterial cell to produce at least one commodity chemical.

27. The isolated bacterial cell of claim 26, wherein the bacterial cell produces the at least one commodity chemical.

28. The isolated bacterial cell of claim 27, wherein the bacterial cell continually produces the at least one commodity chemical under diurnal conditions.

29. The isolated bacterial cell of claim 26, wherein the commodity chemical is selected from the group consisting of a polymer, 2,3-butanediol, 1,3-propandiol, 1,4-butanediol, polyhydroxyalkanoate, polyhydroxybutyrate, isoprene, lactate, succinate, glutamate, citrate, malate, 3-hydroxypropionate, ascorbate, sorbitol, an amino acid, hydroxybutyrate, a carotenoid, lycopene, β-carotene, a pharmaceutical intermediate, a polyketide, a statin, an omega-3 fatty acid, an isoprenoid, a steroid, an antibiotic, erythromycin, a soprenoid, a steroid, erythromycin, and combinations thereof.

30. The isolated bacterial cell of claim 26, wherein the commodity chemical is a biofuel selected from the group consisting of an alcohol, ethanol, propanol, isopropanol, acetone, butanol, isobutanol, 2-methyl-1-butanol, 3-methyl-1-butanol, phenylethanol, a fatty alcohol, isopentenol, an aldehyde, acetylaldehyde, propionaldehyde, butryaldehyde, isobutyraldehyde, 2-methyl-1-butanal, 3-methyl-1-butanal, phenylacetaldehyde, a fatty aldehyde, a hydrocarbon, an alkane, an alkene, an isoprenoids, a fatty acid, a wax ester, an ethyl ester, hydrogen, and combinations thereof.

31. The isolated bacterial cell of any one of claims 1-22, wherein the bacterial cell is selected from the group consisting of cyanobacteria, Acaryochloris, Anabaena, Arthrospira, Cyanothece, Gleobacter, Microcystis, Nostoc, Prochlorococcus, Synechococcus, Synechococcus elongatus, S. elongatus PCC7942, Synechocystis, Thermosynechococcus, Trichodesmium, purple sulfur bacteria, Chromatiaceae, Ectothiorhodospiraceae, purple non-sulfur bacteria, Acetobacteraceae, Bradyrhizobiaceae, Comamonadaceae, Hyphomicrobiaceae, Rhodobacteraceae, Rhodobiaceae, Rhodocyclaceae, Rhodospirillaceae, green non-sulfur bacteria, and Chloroflexaceae.

32. A method of increasing bacterial cell growth, comprising:
providing a bacterial cell of a photoautotrophic species comprising a recombinant polynucleotide encoding a sugar transporter protein; and
culturing the bacterial cell with a sugar substrate under conditions whereby the recombinant polynucleotide is expressed,
wherein expression of the recombinant polynucleotide results in transport of the sugar substrate into the bacterial cell to increase cell growth on sugar under dark or diurnal conditions as compared to cell growth of a corresponding photoautotrophic bacterial cell lacking the recombinant polynucleotide.

33. A method of increasing bacterial cell density under dark or diurnal conditions, comprising:
providing a bacterial cell of a photoautotrophic species comprising a recombinant polynucleotide encoding a sugar transporter protein; and
culturing the bacterial cell with a sugar substrate under conditions whereby the recombinant polynucleotide is expressed,
wherein expression of the recombinant polynucleotide results in transport of the sugar substrate into the bacterial cell to increase cell density under dark or diurnal conditions as compared to a corresponding photoautotrophic bacterial cell lacking the recombinant polynucleotide.

34. A method of increasing bacterial biomass production under dark or diurnal conditions, comprising:
providing a bacterial cell of a photoautotrophic species comprising a recombinant polynucleotide encoding a sugar transporter protein; and
culturing the bacterial cell with a sugar substrate under conditions whereby the recombinant polynucleotide is expressed,
wherein expression of the recombinant polynucleotide results in transport of the sugar substrate into the bacterial cell to increase biomass production under dark or diurnal conditions as compared to a corresponding photoautotrophic bacterial cell lacking the recombinant polynucleotide.

35. A method of producing at least one commodity chemical, comprising:
providing a bacterial cell of a photoautotrophic species comprising a recombinant polynucleotide encoding a sugar transporter protein;
culturing the bacterial cell with a sugar substrate under conditions whereby the recombinant polynucleotide is expressed and at least one commodity chemical is produced; and
collecting the at least one commodity chemical,
wherein expression of the recombinant polynucleotide results in transport of the sugar substrate into the bacterial cell.

36. The method of any one of claims 32-35, wherein the recombinant polynucleotide encodes a sugar transporter protein selected from the group consisting of a hexose sugar transporter protein, a galactose transporter protein, a glucose transporter protein, a fructose transporter protein, a mannose transporter protein, a Major Facilitator Superfamily (MFS) transporter protein, an ATP-Binding Cassette Superfamily (ABC) transporter protein, a Phosphotransferase System (PTS) transporter protein, a disaccharide sugar transporter protein, a sucrose transporter protein, a lactose transporter protein, a lactulose transporter protein, a maltose transporter protein, a trehalose transporter protein, a cellobiose transporter protein, a pentose transporter protein, a xylose transporter protein, an arabinose transporter protein, a ribose transporter protein, a ribulose transporter protein, and a xylulose transporter protein.

37. The method claim 36, wherein the bacterial cell is cultured with a sugar selected from the group consisting of a hexose, galactose, glucose, fructose, mannose, a disaccharide, sucrose, lactose, lactulose, maltose, trehalose, cellobiose, a pentose, xylose, arabinose, ribose, ribulose, and xylulose.

38. The method of any one of claims 32-35, wherein the recombinant polynucleotide encodes a galactose transporter protein.

39. The method of claim 38, wherein the galactose transporter protein is selected from the group consisting of a bacterial galP transporter protein, a eukaryotic galP transporter protein, a fungal galP transporter protein, a mammalian galP transporter protein, a bacterial MFS transporter protein, a eukaryotic MFS transporter protein, a fungal MFS transporter protein, a mammalian MFS transporter protein, a bacterial PTS transporter protein, a eukaryotic PTS transporter protein, a fungal PTS transporter protein, and a mammalian PTS transporter protein.

40. The method of claim 38, wherein the galactose transporter protein is an E. coli galP transporter protein.

41. The method of claim 38, wherein the galactose transporter protein transports glucose into the bacterial cell.

42. The method of claim 38, wherein the bacterial cell is cultured with glucose.

43. The method of any one of claims 32-35, wherein the recombinant polynucleotide encodes a disaccharide sugar transporter protein.

44. The method of claim 43, wherein the disaccharide sugar transporter protein is selected from the group consisting of a sucrose transporter protein, a lactose transporter protein, a lactulose transporter protein, a maltose transporter protein, a trehalose transporter protein, a cellobiose transporter protein, and a homolog thereof.

45. The method of claim 43, wherein the disaccharide sugar transporter protein is a sucrose transporter protein.

46. The method of claim 45, wherein the sucrose transporter protein is selected from the group consisting of an E. coli CscB sucrose transporter protein, a B. subtilis SacP transporter protein, a Brassica napus Sut1 transporter protein, a Juglans regia Sut1 transporter protein, an Arabidopsis thaliana Suc6 transporter protein, an Arabidopsis thaliana SUT4 transporter protein, a Drosophila melanogaster Slc45-1 transporter protein, and a Dickeya dadantii ScrA transporter protein.

47. The method of claim 45, wherein the sucrose transporter protein is an E. coli CscB sucrose transporter protein.

48. The method of claim 43, wherein the bacterial cell further comprises at least one additional recombinant polynucleotide encoding a fructokinase protein.

49. The method of claim 48, wherein the fructokinase protein is selected from the group consisting of an E. coli CscK fructokinase protein, a Lycopersicon esculentum Frk1 fructokinase protein, a Lycopersicon esculentum Frk2 fructokinase protein, a H. sapiens KHK fructokinase protein, an A. thaliana FLN-1 fructokinase protein, an A. thaliana and FLN-2 fructokinase protein, a Yersinia pestis biovar Microtus str. 91001 NagC1 fructokinase protein, a Yersinia pseudotuberculosis YajF fructokinase protein, and a Natronomonas pharaonis Suk fructokinase protein.

50. The method of claim 48, wherein the fructokinase protein is an E. coli CscK fructokinase protein.

51. The method of claim 43, wherein the bacterial cell further comprises the proteins necessary to convert the disaccharide sugar into its corresponding monosaccharides.

52. The method of claim 43, wherein the bacterial cell is cultured with a sugar selected from the group consisting of a disaccharide sugar, sucrose, lactose, lactulose, maltose, trehalose, and cellobiose.

53. The method of any one of claims 32-35, wherein the recombinant polynucleotide encodes a xylose transporter protein.

54. The method of claim 53, wherein the recombinant polynucleotide encodes a xylose transporter protein selected from the group consisting of an E. coli XylE xylose transporter protein, an E. coli xylF/xylG/xylH ABC xylose transporter protein, a Candida intermedia Gxf1 transporter protein, a Pichia stipitis Sut1 transporter protein, and an A. thaliana At5g59250transporter protein.

55. The method of claim 53, wherein the xylose transporter protein is an E. coli XylE xylose transporter protein.

56. The method of claim 53, wherein the bacterial cell further comprises at least one additional recombinant polynucleotide encoding a xylose isomerase.

57. The method of claim 53, wherein the bacterial cell further comprises at least one additional recombinant polynucleotide encoding a xylulokinase.

58. The method of claim 53, wherein the bacterial cell further comprises a second recombinant polynucleotide encoding a xylose isomerase and a third recombinant polynucleotide encoding a xylulokinase.

59. The method of claim 53, wherein the recombinant polynucleotide further encodes a xylose isomerase and a xylulokinase.

60. The method of claim 56, wherein the xylose isomerase is selected from the group consisting of an E. coli XylA xylose isomerase, an A. thaliana AT5G57655 xylose isomerase, an Aspergillus niger XyrA xylose isomerase, and a Hypocrea jecorina Xyl1 xylose isomerase.

61. The method of claim 56, wherein the xylose isomerase is an E. coli XylA xylose isomerase.

62. The method of claim 57, wherein the xylulokinase is selected from the group consisting of an E. coli XylB xylulokinase, an Arabidopsis thaliana XK-1 xylulokinase, an Arabidopsis thaliana XK-2 xylulokinase, an E. coli LynK xylulokinase, a Streptomyces coelicolor SCO1170 xylulokinase, a Pseudomonas aeruginosa MtlY xylulokinase, a Yersinia pseudotuberculosis SgbK xylulokinase, and an E. coli AtlK xylulokinase.

63. The method of claim 57, wherein the xylulokinase is an E. coli XylB xylulokinase.

64. The method of any one of claims 32-35, wherein the recombinant polynucleotide and/or at least one additional recombinant polynucleotide is stably integrated into the genome of the bacterial cell.

65. The method of any one of claims 32-35, wherein the bacterial cell further comprises at least one additional recombinant polynucleotide encoding a second sugar transport protein, wherein expression of the second sugar transporter protein results in transport of a second sugar substrate into the bacterial cell.

66. The method of any one of claims 32-35, wherein the bacterial cell continually produces the at least one commodity chemical.

67. The method of claim 66, wherein the at least one commodity chemical is continually produced 24 hours a day.

68. The method of claim 66, wherein the commodity chemical is selected from the group consisting of a polymer, 2,3-butanediol, 1,3-propandiol, 1,4-butanediol, polyhydroxyalkanoate, polyhydroxybutyrate, isoprene, lactate, succinate, glutamate, citrate, malate, 3-hydroxypropionate, ascorbate, sorbitol, an amino acid, hydroxybutyrate, a carotenoid, lycopene, β-carotene, a pharmaceutical intermediate, a polyketide, a statin, an omega-3 fatty acid, an isoprenoid, a steroid, an antibiotic, erythromycin, a soprenoid, a steroid, erythromycin, a biofuel, and combinations thereof.

69. The method of claim 66, wherein the commodity chemical is a biofuel selected from the group consisting of an alcohol, ethanol, propanol, isopropanol, acetone, butanol, isobutanol, 2-methyl-1-butanol, 3-methyl-1-butanol, phenylethanol, a fatty alcohol, isopentenol, an aldehyde, acetylaldehyde, propionaldehyde, butryaldehyde, isobutyraldehyde, 2-methyl-1-butanal, 3-methyl-1-butanal, phenylacetaldehyde, a fatty aldehyde, a hydrocarbon, an alkane, an alkene, an isoprenoids, a fatty acid, a wax ester, an ethyl ester, hydrogen, and combinations thereof.

70. The method of any one of claims 32-35, wherein the bacterial cell is selected from the group consisting of cyanobacteria, Acaryochloris, Anabaena, Arthrospira, Cyanothece, Gleobacter, Microcystis, Nostoc, Prochlorococcus, Synechococcus, Synechococcus elongatus, S. elongatus PCC7942, Synechocystis, Thermosynechococcus, Trichodesmium, purple sulfur bacteria, Chromatiaceae, Ectothiorhodospiraceae, purple non-sulfur bacteria, Acetobacteraceae, Bradyrhizobiaceae, Comamonadaceae, Hyphomicrobiaceae, Rhodobacteraceae, Rhodobiaceae, Rhodocyclaceae, Rhodospirillaceae, green non-sulfur bacteria, and Chloroflexaceae.


SEQUENCE LISTING (41 sequences)

SEQ ID NO: 1 - DNA, 1395 bp, E. coli
atgcctgacgctaaaaaacaggggcggtcaaacaaggcaatgacgtttttcgtctgcttccttgccgctctggcgggattactctttggcctggatatcggtgtaattgctggcgcactgccgtttattgcagatgaattccagattacttcgcacacgcaagaatgggtcgtaagctccatgatgttcggtgcggcagtcggtgcggtgggcagcggctggctctcctttaaactcgggcgcaaaaagagcctgatgatcggcgcaattttgtttgttgccggttcgctgttctctgcggctgcgccaaacgttgaagtactgattctttcccgcgttctactggggctggcggtgggtgtggcctcttataccgcaccgctgtacctctctgaaattgcgccggaaaaaattcgtggcagtatgatctcgatgtatcagttgatgatcactatcgggatcctcggtgcttatctttctgataccgccttcagctacaccggtgcatggcgctggatgctgggtgtgattatcatcccggcaattttgctgctgattggtgtcttcttcctgccagacagcccacgttggtttgccgccaaacgccgttttgttgatgccgaacgcgtgctgctacgcctgcgtgacaccagcgcggaagcgaaacgcgaactggatgaaatccgtgaaagtttgcaggttaaacagagtggctgggcgctgtttaaagagaacagcaacttccgccgcgcggtgttccttggcgtactgttgcaggtaatgcagcaattcaccgggatgaacgtcatcatgtattacgcgccgaaaatcttcgaactggcgggttataccaacactaccgagcaaatgtgggggaccgtgattgtcggcctgaccaacgtacttgccacctttatcgcaatcggccttgttgaccgctggggacgtaaaccaacgctaacgctgggcttcctggtgatggctgctggcatgggcgtactcggtacaatgatgcatatcggtattcactctccgtcggcgcagtatttcgccatcgccatgctgctgatgtttattgtcggttttgccatgagtgccggtccgctgatttgggtactgtgctccgaaattcagccgctgaaaggccgcgattttggcatcacctgctccactgccaccaactggattgccaacatgatcgttggcgcaacgttcctgaccatgctcaacacgctgggtaacgccaacaccttctgggtgtatgcggctctgaacgtactgtttatcctgctgacattgtggctggtaccggaaaccaaacacgtttcgctggaacatattgaacgtaatctgatgaaaggtcgtaaactgcgcgaaataggcgctcacgattaa

SEQ ID NO: 2 - protein, 464 aa, E. coli
MPDAKKQGRSNKAMTFFVCFLAALAGLLFGLDIGVIAGALPFIADEFQITSHTQEWVVSSMMFGAAVGAVGSGWLSFKLGRKKSLMIGAILFVAGSLFSAAAPNVEVLILSRVLLGLAVGVASYTAPLYLSEIAPEKIRGSMISMYQLMITIGILGAYLSDTAFSYTGAWRWMLGVIIIPAILLLIGVFFLPDSPRWFAAKRRFVDAERVLLRLRDTSAEAKRELDEIRESLQVKQSGWALFKENSNFRRAVFLGVLLQVMQQFTGMNVIMYYAPKIFELAGYTNTTEQMWGTVIVGLTNVLATFIAIGLVDRWGRKPTLTLGFLVMAAGMGVLGTMMHIGIHSPSAQYFAIAMLLMFIVGFAMSAGPLIWVLCSEIQPLKGRDFGITCSTATNWIANMIVGATFLTMLNTLGNANTFWVYAALNVLFILLTLWLVPETKHVSLEHIERNLMKGRKLREIGAHD

SEQ ID NO: 3 - DNA, 1407 bp, Synechocystis sp.
atgaatccctcctcttctccttcccaatctacggctaacgttaagtttgtcctgctgatttcgggggtagcagccctgggggggttcctgtttggctttgacactgcggtgatcaatggggcggtggcggccctacaaaaacattttcagacggacagtcttttaacaggtttatctgtatccttagctctgttgggatcagcactgggagcctttggggcgggaccgatcgccgatcgccatgggcggattaaaacgatgattttagcggcggtgctgttcaccctcagttccattgggtcgggtttacctttcaccatttgggattttattttttggcgggtgttggggggcattggggtgggggccgctagcgttattgccccggcctacattgcggaagtgtcgccggcccatctgcgggggcgtttaggatctttgcaacagttggccattgtttctggcattttcattgccctgctcagtaattggtttattgctttgatggcggggggatcggcccaaaatccctggttgttcggtgcggcggcctggcgttggatgttctggacagagctaattcccgccctgctctatggagtttgcgctttcctgatccccgaatctccccggtatttagtcgcccaagggcaaggggaaaaagcggcggctattttgtggaaagtggaagggggagacgtgcccagtcgcattgaggaaatccaggcaacggttagtctcgaccataaaccccggtttagcgatctgctcagtcgtcggggaggattattgcccattgtctggattggtatggggttgtcggcactacaacagtttgttggcattaacgtaattttttattacagtagcgtgctctggcgatcggtgggttttaccgaagaaaagtctctgttaatcacggtaatcactggttttatcaatatcctcaccaccctagtggcgatcgcctttgtggataaatttggccgtaagcctttgttgctcatgggctccattggtatgaccattaccttgggcatcctttccgtggtgtttgggggagcaacggtggttaatggccaacccaccctgacgggggccgctgggataattgctttggtgacagccaatctttatgtatttagttttggtttttcttgggggcccattgtttgggtcttgctgggggaaatgtttaataacaaaattcgagcggcggccctatcggtggcggcgggggtacagtggattgctaactttattatttccactacttttccgcccctattggatacggtgggtttgggccccgcctatggtttatatgccacttcagcggccatttcaattttctttatctggttttttgtgaaggaaactaagggtaaaactctggagcaaatgtga

SEQ ID NO: 4 - protein, 468 aa, Synechocystis sp.
MNPSSSPSQSTANVKFVLLISGVAALGGFLFGFDTAVINGAVAALQKHFQTDSLLTGLSVSLALLGSALGAFGAGPIADRHGRIKTMILAAVLFTLSSIGSGLPFTIWDFIFWRVLGGIGVGAASVIAPAYIAEVSPAHLRGRLGSLQQLAIVSGIFIALLSNWFIALMAGGSAQNPWLFGAAAWRWMFWTELIPALLYGVCAFLIPESPRYLVAQGQGEKAAAILWKVEGGDVPSRIEEIQATVSLDHKPRFSDLLSRRGGLLPIVWIGMGLSALQQFVGINVIFYYSSVLWRSVGFTEEKSLLITVITGFINILTTLVAIAFVDKFGRKPLLLMGSIGMTITLGILSVVFGGATVVNGQPTLTGAAGIIALVTANLYVFSFGFSWGPIVWVLLGEMFNNKIRAAALSVAAGVQWIANFIISTTFPPLLDTVGLGPAYGLYATSAAISIFFIWFFVKETKGKTLEQM

SEQ ID NO: 5 - DNA, 1479 bp, H. sapiens
atggaacccagctccaagaaattgaccggacgcctgatgctggccgttggaggggccgtgctgggctcgctgcagtttggctacaacaccggcgtgatcaatgcgccgcagaaagtgattgaagaattttacaaccaaacctgggtccatcgctacggcgagagcatcctgcccacaaccctcaccaccctgtggagcctgagcgtcgccatttttagtgtgggaggtatgatcggcagcttctccgtgggcttgttcgtcaatcgctttggtcggcgcaactccatgctcatgatgaacctgttggcctttgtgtctgcggtcctcatgggcttcagcaaactgggaaagtcgtttgaaatgctgatcctcggtcgcttcattatcggcgtgtactgtggcttgactaccggctttgttcctatgtacgtgggggaggtgtcgcccaccgcactccgcggtgcgctgggtacgttgcatcaattgggcatcgtggtgggcattctcatcgcccaggtgtttggcctggatagcatcatgggtaacaaagatttgtggccgctgctcctgagtatcattttcatccccgcactgttgcaatgtatcgtgctccctttttgcccggaatccccccgttttctcctcatcaaccgcaacgaagaaaaccgcgccaaaagcgtgctcaaaaagttgcgagggaccgccgatgtgactcacgacttgcaggagatgaaagaggagagccgtcagatgatgcgcgagaagaaggtgacaattctggaactgttccgcagccctgcgtaccgccaaccaatcttgattgcagtcgtgctgcaactcagccagcagttgagcggcattaacgctgtcttttactattccacctcgatctttgaaaaagcaggcgtgcagcaacccgtctacgccaccattggatcggggatcgtgaataccgcttttaccgttgtgtcgctgtttgtcgttgaacgagctggacggcgaactctccacctgattggtctggccgggatggctggatgcgccatcctgatgaccattgcactggccctcctggaacaactgccctggatgagctacctctctattgttgccatctttggcttcgtggcgttctttgaggttggtccgggtccaatcccatggtttatcgtggctgaactctttagccagggtccacgtccggctgcgattgctgtggcaggtttttcgaattggacgagtaacttcatcgtcggcatgtgttttcaatacgtcgaacagctctgtggcccatacgtgtttatcatcttcaccgtcttgctcgtcctgttctttatctttacatacttcaaagtgcccgaaaccaaagggcggacctttgatgaaatcgccagcggctttcggcagggaggagccagccagagcgataagaccccagaggagttgtttcacccattgggggctgatagccaagtgtga

SEQ ID NO: 6 - protein, 492 aa, H. sapiens
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTAFRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 7 - DNA, 1476 bp, E. coli
atgaatacccagtataattccagttatatattttcgattaccttagtcgctacattaggtggtttattatttggctacgacaccgccgttatttccggtactgttgagtcactcaataccgtctttgttgctccacaaaacttaagtgaatccgctgccaactccctgttagggttttgcgtggccagcgctctgattggttgcatcatcggcggtgccctcggtggttattgcagtaaccgcttcggtcgtcgtgattcacttaagattgctgctgtcctgttttttatttctggtgtaggttctgcctggccagaacttggttttacctctataaacccggacaacactgtgcctgtttatctggcaggttatgtcccggaatttgttatttatcgcattattggcggtattggcgttggtttagcctcaatgctctcgccaatgtatattgcggaactggctccagctcatattcgcgggaaactggtctcttttaaccagtttgcgattattttcgggcaacttttagtttactgcgtaaactattttattgcccgttccggtgatgccagctggctgaatactgacggctggcgttatatgtttgcctcggaatgtatccctgcactgctgttcttaatgctgctgtataccgtgccagaaagtcctcgctggctgatgtcgcgcggcaagcaagaacaggcggaaggtatcctgcgcaaaattatgggcaacacgcttgcaactcaggcagtacaggaaattaaacactccctggatcatggccgcaaaaccggtggtcgtctgctgatgtttggcgtgggcgtgattgtaatcggcgtaatgctctccatcttccagcaatttgtcggcatcaatgtggtgctgtactacgcgccggaagtgttcaaaacgctgggggccagcacggatatcgcgctgttgcagaccattattgtcggagttatcaacctcaccttcaccgttctggcaattatgacggtggataaatttggtcgtaagccactgcaaattatcggcgcactcggaatggcaatcggtatgtttagcctcggtaccgcgttttacactcaggcaccgggtattgtggcgctactgtcgatgctgttctatgttgccgcctttgccatgtcctggggtccggtatgctgggtactgctgtcggaaatcttcccgaatgctattcgtggtaaagcgctggcaatcgcggtggcggcccagtggctggcgaactacttcgtctcctggaccttcccgatgatggacaaaaactcctggctggtggcccatttccacaacggtttctcctactggatttacggttgtatgggcgttctggcagcactgtttatgtggaaatttgtcccggaaaccaaaggtaaaacccttgaggagctggaagcgctctgggaaccggaaacgaagaaaacacaacaaactgctacgctgtaa

SEQ ID NO: 8 - protein, 491 aa, E. coli
MNTQYNSSYIFSITLVATLGGLLFGYDTAVISGTVESLNTVFVAPQNLSESAANSLLGFCVASALIGCIIGGALGGYCSNRFGRRDSLKIAAVLFFISGVGSAWPELGFTSINPDNTVPVYLAGYVPEFVIYRIIGGIGVGLASMLSPMYIAELAPAHIRGKLVSFNQFAIIFGQLLVYCVNYFIARSGDASWLNTDGWRYMFASECIPALLFLMLLYTVPESPRWLMSRGKQEQAEGILRKIMGNTLATQAVQEIKHSLDHGRKTGGRLLMFGVGVIVIGVMLSIFQQFVGINVVLYYAPEVFKTLGASTDIALLQTIIVGVINLTFTVLAIMTVDKFGRKPLQIIGALGMAIGMFSLGTAFYTQAPGIVALLSMLFYVAAFAMSWGPVCWVLLSEIFPNAIRGKALAIAVAAQWLANYFVSWTFPMMDKNSWLVAHFHNGFSYWIYGCMGVLAALFMWKFVPETKGKTLEELEALWEPETKKTQQTATL

SEQ ID NO: 9 - DNA, 1323 bp, E. coli
atgcaagcctattttgaccagctcgatcgcgttcgttatgaaggctcaaaatcctcaaacccgttagcattccgtcactacaatcccgacgaactggtgttgggtaagcgtatggaagagcacttgcgttttgccgcctgctactggcacaccttctgctggaacggggcggatatgtttggtgtgggggcgtttaatcgtccgtggcagcagcctggtgaggcactggcgttggcgaagcgtaaagcagatgtcgcatttgagtttttccacaagttacatgtgccattttattgcttccacgatgtggatgtttcccctgagggcgcgtcgttaaaagagtacatcaataattttgcgcaaatggttgatgtcctggcaggcaagcaagaagagagcggcgtgaagctgctgtggggaacggccaactgctttacaaaccctcgctacggcgcgggtgcggcgacgaacccagatcctgaagtcttcagctgggcggcaacgcaagttgttacagcgatggaagcaacccataaattgggcggtgaaaactatgtcctgtggggcggtcgtgaaggttacgaaacgctgttaaataccgacttgcgtcaggagcgtgaacaactgggccgctttatgcagatggtggttgagcataaacataaaatcggtttccagggcacgttgcttatcgaaccgaaaccgcaagaaccgaccaaacatcaatatgattacgatgccgcgacggtctatggcttcctgaaacagtttggtctggaaaaagagattaaactgaacattgaagctaaccacgcgacgctggcaggtcactctttccatcatgaaatagccaccgccattgcgcttggcctgttcggttctgtcgacgccaaccgtggcgatgcgcaactgggctgggacaccgaccagttcccgaacagtgtggaagagaatgcgctggtgatgtatgaaattctcaaagcaggcggtttcaccaccggtggtctgaacttcgatgccaaagtacgtcgtcaaagtactgataaatatgatctgttttacggtcatatcggcgcgatggatacgatggcactggcgctgaaaattgcagcgcgcatgattgaagatggcgagctggataaacgcatcgcgcagcgttattccggctggaatagcgaattgggccagcaaatcctgaaaggccaaatgtcactggcagatttagccaaatatgctcaggaacatcatttgtctccggtgcatcagagtggtcgccaggaacaactggaaaatctggtaaaccattatctgttcgacaaataa

SEQ ID NO: 10 - protein, 440 aa, E. coli
MQAYFDQLDRVRYEGSKSSNPLAFRHYNPDELVLGKRMEEHLRFAACYWHTFCWNGADMFGVGAFNRPWQQPGEALALAKRKADVAFEFFHKLHVPFYCFHDVDVSPEGASLKEYINNFAQMVDVLAGKQEESGVKLLWGTANCFTNPRYGAGAATNPDPEVFSWAATQVVTAMEATHKLGGENYVLWGGREGYETLLNTDLRQEREQLGRFMQMVVEHKHKIGFQGTLLIEPKPQEPTKHQYDYDAATVYGFLKQFGLEKEIKLNIEANHATLAGHSFHHEIATAIALGLFGSVDANRGDAQLGWDTDQFPNSVEENALVMYEILKAGGFTTGGLNFDAKVRRQSTDKYDLFYGHIGAMDTMALALKIAARMIEDGELDKRIAQRYSGWNSELGQQILKGQMSLADLAKYAQEHHLSPVHQSGRQEQLENLVNHYLFDK

SEQ ID NO: 11 - DNA, 1455 bp, E. coli
atgtatatcgggatagatcttggcacctcgggcgtaaaagttattttgctcaacgagcagggtgaggtggttgctgcgcaaacggaaaagctgaccgtttcgcgcccgcatccactctggtcggaacaagacccggaacagtggtggcaggcaactgatcgcgcaatgaaagctctgggcgatcagcattctctgcaggacgttaaagcattgggtattgccggccagatgcacggagcaaccttgctggatgctcagcaacgggtgttacgccctgccattttgtggaacgacgggcgctgtgcgcaagagtgcactttgctggaagcgcgagttccgcaatcgcgggtgattaccggcaacctgatgatgcccggatttactgcgcctaaattgctatgggttcagcggcatgagccggagatattccgtcaaatcgacaaagtattattaccgaaagattacttgcgtctgcgtatgacgggggagtttgccagcgatatgtctgacgcagctggcaccatgtggctggatgtcgcaaagcgtgactggagtgacgtcatgctgcaggcttgcgacttatctcgtgaccagatgcccgcattatacgaaggcagcgaaattactggtgctttgttacctgaagttgcgaaagcgtggggtatggcgacggtgccagttgtcgcaggcggtggcgacaatgcagctggtgcagttggtgtgggaatggttgatgctaatcaggcaatgttatcgctggggacgtcgggggtctattttgctgtcagcgaagggttcttaagcaagccagaaagcgccgtacatagcttttgccatgcgctaccgcaacgttggcatttaatgtctgtgatgctgagtgcagcgtcgtgtctggattgggccgcgaaattaaccggcctgagcaatgtcccagctttaatcgctgcagctcaacaggctgatgaaagtgccgagccagtttggtttctgccttatctttccggcgagcgtacgccacacaataatccccaggcgaagggggttttctttggtttgactcatcaacatggccccaatgaactggcgcgagcagtgctggaaggcgtgggttatgcgctggcagatggcatggatgtcgtgcatgcctgcggtattaaaccgcaaagtgttacgttgattgggggcggggcgcgtagtgagtactggcgtcagatgctggcggatatcagcggtcagcagctcgattaccgtacggggggggatgtggggccagcactgggcgcagcaaggctggcgcagatcgcggcgaatccagagaaatcgctcattgaattgttgccgcaactaccgttagaacagtcgcatctaccagatgcgcagcgttatgccgcttatcagccacgacgagaaacgttccgtcgcctctatcagcaacttctgccattaatggcgtaa

SEQ ID NO: 12 - protein, 484 aa, E. coli
MYIGIDLGTSGVKVILLNEQGEVVAAQTEKLTVSRPHPLWSEQDPEQWWQATDRAMKALGDQHSLQDVKALGIAGQMHGATLLDAQQRVLRPAILWNDGRCAQECTLLEARVPQSRVITGNLMMPGFTAPKLLWVQRHEPEIFRQIDKVLLPKDYLRLRMTGEFASDMSDAAGTMWLDVAKRDWSDVMLQACDLSRDQMPALYEGSEITGALLPEVAKAWGMATVPVVAGGGDNAAGAVGVGMVDANQAMLSLGTSGVYFAVSEGFLSKPESAVHSFCHALPQRWHLMSVMLSAASCLDWAAKLTGLSNVPALIAAAQQADESAEPVWFLPYLSGERTPHNNPQAKGVFFGLTHQHGPNELARAVLEGVGYALADGMDVVHACGIKPQSVTLIGGGARSEYWRQMLADISGQQLDYRTGGDVGPALGAARLAQIAANPEKSLIELLPQLPLEQSHLPDAQRYAAYQPRRETFRRLYQQLLPLMA

SEQ ID NO: 13 - DNA, 1248 bp, E. coli
atggcactgaatattccattcagaaatgcgtactatcgttttgcatccagttactcatttctcttttttatttcctggtcgctgtggtggtcgttatacgctatttggctgaaaggacatctagggttgacagggacggaattaggtacactttattcggtcaaccagtttaccagcattctatttatgatgttctacggcatcgttcaggataaactcggtctgaagaaaccgctcatctggtgtatgagtttcatcctggtcttgaccggaccgtttatgatttacgtttatgaaccgttactgcaaagcaatttttctgtaggtctaattctgggggcgctcttttttggcctggggtatctggcgggatgcggtttgcttgacagcttcaccgaaaaaatggcgcgaaattttcatttcgaatatggaacagcgcgcgcctggggatcttttggctatgctattggcgcgttctttgccggcatattttttagtatcagtccccatatcaacttctggttggtctcgctatttggcgctgtatttatgatgatcaacatgtgttttaaagataaggatcaccagtgcgtagcggcggatgcgggaggggtaaaaaaagaggattttatcgcagttttcaaggatcgaaacttctgggttttcgtcatatttattgttgggacgtggtctttctataacatttttgatcaacaactttttcctgtcttttatgcaggtttattcgaatcacacgatgtaggaacgcgcctgtatggttatctcaactcattccaggtggtactcgaagcgctgtgcatggcgattattccgttctttgtgaatcgggtagggccaaaaaatgcattacttatcggtgttgtgattatggcgttgcgtatcctttcctgcgcgctgttcgttaacccctggattatttcattagtgaagctgttacatgccattgaggttccactttgtgtcatatccgtcttcaaatacagcgtggcaaactttgataagcgcctgtcgtcgacgatctttctgattggttttcaaattgccagttcgcttgggattgtgctgctttcaacgccgactgggatactctttgaccacgcaggctaccagacagttttcttcgcaatttcgggtattgtctgcctgatgttgctatttggcattttcttcctgagtaaaaaacgcgagcaaatagttatggaaacgcctgtaccttcagcaatatag

SEQ ID NO: 14 - protein, 415 aa, E. coli
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMCFKDKDHQCVAADAGGVKKEDFIAVFKDRNFWVFVIFIVGTWSFYNIFDQQLFPVFYAGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 15 - DNA, 918 bp, E. coli
  VARIANT 68: n = Any Nucleotide
atgtcagccaaagtatgggttttaggggatgcgggtcgtagatctcttgccagaatcagacgggcggnwtactgccttgtcctggcggcgcgccagctaacgttgccggtgggaatcgccagattaggcggaacaagtgggtttataggtcgggtgggggatgatccttttggtgcattaatgcaaagaacgctgctaactgagggagtcgatatcacgtatctgaagcaagatgaatggcaccggacatccacggtgcttgtcgatctgaacgatcaaggggaacgttcatttacgtttatggtccgccccagtgccgatctttttttagagacgacagacttgccctgctggcgacatggcgaatggttacatctctgttcaattgcgttgtctgccgagccttcgcgtaccagcgcatttactgcgatgacggagatccggcatgccggaggttttgtcagcttcgatcccaatattcgtgaagatctatggcaagacgagcatttgctccgcttgtgtttgcggcaggcgctacaactggcggatgtcgtcaagctctcggaagaagaatggcgacttatcagtggaaaaacacagaacgatcgggatatatgcgccctggcaaaagagtatgagatcgccatgctgttggtgactaaaggtgcagaaggggtggtggtctgttatcgaggacaagttcaccattttgctggaatgtctgtggattgtgtcgatagcacgggggcgggagatgcgttcgttgccgggttactcacaggtctgtcctctacgggattatctacagatgagagagaaatgcgacgaattatcgatctcgctcaacgttgcggagcgcttgcagtaacggcgaaaggggcaatgacagcgctgccatgtcgacaagaactggaatag

SEQ ID NO: 16 - protein, 305 aa, E. coli
  VARIANT 23: Xaa = Any Amino Acid
MSAKVWVLGDAGRRSLARIRRAXYCLVLAARQLTLPVGIARLGGTSGFIGRVGDDPFGALMQRTLLTEGVDITYLKQDEWHRTSTVLVDLNDQGERSFTFMVRPSADLFLETTDLPCWRHGEWLHLCSIALSAEPSRTSAFTAMTEIRHAGGFVSFDPNIREDLWQDEHLLRLCLRQALQLADVVKLSEEEWRLISGKTQNDRDICALAKEYEIAMLLVTKGAEGVVVCYRGQVHHFAGMSVDCVDSTGAGDAFVAGLLTGLSSTGLSTDEREMRRIIDLAQRCGALAVTAKGAMTALPCRQELE

SEQ ID NO: 17 - DNA, 43 bp, Artificial Sequence
  Primer Sequence
ccggaattcaatacccagtataattccagttatatattttcga

SEQ ID NO: 18 - DNA, 38 bp, Artificial Sequence
  Primer Sequence
cgggatccatcctaggttacagcgtagcagtttgttgt

SEQ ID NO: 19 - DNA, 55 bp, Artificial Sequence
  Primer Sequence
cgcctaggaactttaagaaggagatataccatgcaagcctattttgaccagctcg

SEQ ID NO: 20 - DNA, 33 bp, Artificial Sequence
  Primer Sequence
cgggatccttacgccattaatggcagaagttgc

SEQ ID NO: 21 - DNA, 29 bp, Artificial Sequence
  Primer Sequence
atggaattcatgtcagccaaagtatgggt

SEQ ID NO: 22 - DNA, 43 bp, Artificial Sequence
  Primer Sequence
ggatccattgggacgtcacctcctatattgctgaaggtacagg

SEQ ID NO: 23 - DNA, 30 bp, Artificial Sequence
  Primer Sequence
gaggtgacgtcatgacgcaatctcgattgc

SEQ ID NO: 24 - DNA, 30 bp, Artificial Sequence
  Primer Sequence
tagaggatccttaacccagttgccagagtg

SEQ ID NO: 25 - DNA, 29 bp, Artificial Sequence
  Primer Sequence
atggaattcatggcactgaatattccatt

SEQ ID NO: 26 - DNA, 36 bp, Artificial Sequence
  Primer Sequence
ctaacaattgatgcctgacgctaaaaaacaggggcg

SEQ ID NO: 27 - DNA, 39 bp, Artificial Sequence
  Primer Sequence
ctatagatctttaatcgtgagcgcctatttcgcgcagtt

SEQ ID NO: 28 - DNA, 39 bp, Artificial Sequence
  Primer Sequence
ctatctcgagttaatcgtgagcgcctatttcgcgcagtt

SEQ ID NO: 29 - DNA, 31 bp, Artificial Sequence
  Primer Sequence
catgcctgacgctaaaaaacaggggcggtca

SEQ ID NO: 30 - DNA, 59 bp, Artificial Sequence
  Primer Sequence
ttacggccgctgccaccgccgctaccgccatcgtgagcgcctatttcgcgcagtttacg

SEQ ID NO: 31 - DNA, 59 bp, Artificial Sequence
  Primer Sequence
acgatggcggtagcggcggtggcagcggccgtaaaggagaagaacttttcactggagtt

SEQ ID NO: 32 - DNA, 34 bp, Artificial Sequence
  Primer Sequence
cttagcatgctttgtatagttcatccatgccatg

SEQ ID NO: 33 - DNA, 40 bp, Artificial Sequence
  Primer Sequence
ctatgaattccgtaaaggagaagaacttttcactggagtt

SEQ ID NO: 34 - DNA, 43 bp, Artificial Sequence
  Primer Sequence
ctaaggatccttagcatgctttgtatagttcatccatgccatg

SEQ ID NO: 35 - DNA, 30 bp, Artificial Sequence
  Primer Sequence
ggtgctagccaccgtggaaacggatgaagg

SEQ ID NO: 36 - DNA, 31 bp, Artificial Sequence
  Primer Sequence
catttttgtcgacgccgggaagccgatctcg

SEQ ID NO: 37 - DNA, 20 bp, Artificial Sequence
  Primer Sequence
gagtaggtggctacgtctcc

SEQ ID NO: 38 - DNA, 34 bp, Artificial Sequence
  Primer Sequence
ccgctcgagtaccagcgatccgtgtccctactcg

SEQ ID NO: 39 - DNA, 36 bp, Artificial Sequence
  Primer Sequence
cgagcacgcgtcaattgccctaagacagttgtcgtc

SEQ ID NO: 40 - DNA, 20 bp, Artificial Sequence
  Primer Sequence
cgccgaactgtttgaacagc

SEQ ID NO: 41 - DNA, 24 bp, Artificial Sequence
  Primer Sequence
tagtaacctccagccttttttgcc